(19) United States
(12) Patent Application Publication
(10) Pub. No.: US 2025/0154174 A1
(43) Pub. Date: May 15, 2025

(54) BICYCLIC HETEROCYCLES AS MRGPRX2 ANTAGONISTS

(71) Applicant: Incyte Corporation, Wilmington, DE (US)

(72) Inventors: Minh Nguyen, Claymont, DE (US); Hai Fen Ye, Newark, DE (US)

(21) Appl. No.: 18/941,025

(22) Filed: Nov. 8, 2024

Related U.S. Application Data
(60) Provisional application No. 63/548,135, filed on Nov. 10, 2023.

(51) Int. Cl. C07D 519/00; A61K 31/437; A61K 31/4375; A61K 31/4985; A61K 31/519

(52) U.S. Cl. CPC C07D 519/00; A61K 31/437; A61K 31/4375; A61K 31/4985; A61K 31/519

(57) ABSTRACT

The present disclosure relates to bicyclic heterocycles of Formula (I), and pharmaceutical compositions of the same, that are modulators, antagonists, or inhibitors of the G protein-coupled receptor MRGPRX2 and are useful in the treatment of MRGPRX2 dependent conditions such as inflammatory diseases.

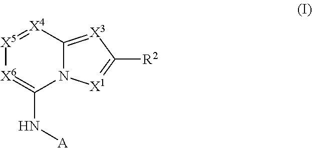

### CROSS-REFERENCE TO RELATED APPLICATIONS

This application claims the benefit of priority to U.S. Provisional Application No. 63/548,135 filed on Nov. 10, 2023, the contents of which are hereby incorporated by reference.

### TECHNICAL FIELD

The present disclosure relates to bicyclic heterocycles, and pharmaceutical compositions of the same, that are modulators, antagonists or inhibitors of the G protein-coupled receptor MRGPRX2 and are useful in the treatment of MRGPRX2 dependent conditions such as inflammatory diseases.

### BACKGROUND

Mas-related G protein-coupled receptor X2 (MRGPRX2) is an orphan, seven transmembrane G protein-coupled receptor that is almost exclusively expressed on connective tissue mast cells. MRGPRX2 belongs to a G protein-coupled receptor subfamily X, comprised of four members X1-X4, specific to humans and primates. MRGPRX2 is a low affinity promiscuous receptor for cyclic and polybasic structure ligands that mediates mast cell degranulation in response to multiple endogenous and exogenous stimuli.

Mast cells constitute an integral part of the human immune system. They are important modulators of inflammatory and physiological processes. MRGPRX2 receptor plays a pivotal role in itch, allergy and inflammation. Activation of the receptor by neuropeptides, antimicrobial host defense peptides as well as numerous FDA-approved drugs leads to mast cell degranulation and release of inflammatory mediators through immunoglobulin-independent pathway (M. Thapaliya, et al., Curr. Allergy Asthma Rep., 2021, 21(1), 3).

Activation of MRGPRX2 receptor drives non-histaminergic itch in chronic refractory pruritus and MRGPRX2 has been implicated in senile itch (A. He, et al. Biomed. Res. Int., 2017, 4790810; J. Meixiong, et al., Immunity, 2019, 50(5), 1163-71). There is an increased MRGPRX2 gene expression on mast cells in the skin of patients with severe chronic urticaria (hives) (H. Ali, J. Immunobiol., 2016, 1(4), 115; D. Fujisawa, et al., J. Allergy Clin. Immunol. 2014, 134(3), 622-33). Additionally, activation of MRGPRX2 by elevated levels of proadrenomedullin N-terminal 20 peptide (PAMP1-20) in the skin of patients with allergic contact dermatitis (ACD) leads to intensely itchy eczematous skin rash (J. Meixiong, et al., Immunity, 2019, 50(5):1163-71). MRGPRX2 is also involved in the pathogenesis of acne rosacea where dysregulation of the host defense mechanism due to excessive LL-37 antimicrobial peptide production leads to enhanced mast cell activation through MRGPRX2 (H. Ali, Adv. Immunol., 2017, 136, 123-62). MRGPRX2 is also implicated in systemic mastocytosis and in neurogenic inflammation, pain and itch. Substance P released from nerve endings and directly from mast cells in sickle cell anemia patients activates mast cells via MRGPRX2 causing painful crisis (H. Subramanian, et al., J. Allergy Clin. Immunol., 2016, 138(3), 700-10).

The role of MRGPRX2 in mast cell biology is further supported by the fact that naturally occurring missense MRGPRX2 variants: G165E, D184H, W243R, and H259Y inhibit mast cell degranulation in response to endogenous neuropeptides and drugs (I. Alkanfari, et al., J. Immuol., 2018, 201(2), 343-49).

Taken together, these findings suggest that MRGPRX2 plays a critical role in itch, pain, and inflammation. Potential disease indications for MRGPRX2 antagonist encompass chronic urticaria and pruritus (hives/itch), acne rosacea, and systemic mastocytosis. These clinical indications present high unmet medical need, particularly in antihistamine-refractory patients. Therefore, targeting of MRGPRX2 receptor can be useful in the clinical treatment of mast-cell mediated diseases.

Compounds that modulate MRGPRX2 are discussed in WO2006066599A2, WO2008052072A2, WO2020223255A1, WO2021092240A1, WO2021092262A1, WO2021092264A1, WO2022067094A1, WO2022073904A1, WO2022073905A1, WO2022087083A1, WO2022111473A1, WO2022125636A1, WO2022140520A1, WO2022152852A1, WO2022152853A1, and WO2023039448A1.

There remains a need for new compounds that are effective as modulators, antagonists or inhibitors of MRGPRX2.

### SUMMARY

The present disclosure is directed to compounds having Formula (I):

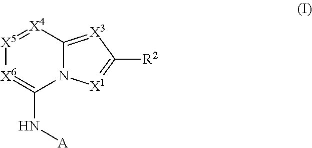

or pharmaceutically acceptable salts thereof, wherein constituent variables are defined herein.

The present disclosure is further directed to pharmaceutical compositions comprising a compound of Formula (I), or a pharmaceutically acceptable salt thereof, and at least one pharmaceutically acceptable carrier.

The present disclosure is further directed to methods of modulating such as by antagonizing or inhibiting MRGPRX2 protein comprising contacting the protein with a compound of Formula (I), or a pharmaceutically acceptable salt thereof.

The present disclosure is further directed to a method of treating MRGPRX2 dependent conditions, comprising administering a compound of Formula (I), or a pharmaceutically acceptable salt thereof, to a patient in need thereof. The present disclosure is further directed to the use of compounds of Formula (I) and pharmaceutically acceptable salts thereof in the preparation of a medicament for use in therapy. The present disclosure is further directed to compounds of Formula (I) and pharmaceutically acceptable salts thereof for use in therapy.

### DETAILED DESCRIPTION

### I. Definitions

For the terms “e.g.” and “such as,” and grammatical equivalents thereof, the phrase “and without limitation” is understood to follow unless explicitly stated otherwise.

The singular forms “a,” “an,” and “the” include plural referents unless the context clearly dictates otherwise.

The term “about” means “approximately” (e.g., plus or minus approximately 10% of the indicated value).

The terms “ambient temperature” and “room temperature” are understood in the art, and refer generally to a temperature, e.g., a reaction temperature, that is about the temperature of the room in which the reaction is carried out, e.g., a temperature from about 20° C. to about 30° C.

The following abbreviations may be used herein: br (broad); BSA (bovine serum albumin); BTK (Bruton tyrosine kinase); CHO-K1 (Chinese hamster ovary); CTMCs (connective tissue-type mast cells); CYP (cytochrome P450); d (doublet); dd (doublet of doublets); DCM (dichloromethane); DIPEA (N,N-diisopropylethylamine); DMEM (Dulbecco's Modified Eagle Medium); DMF (N,N-dimethylformamide); DMSO (dimethylsulfoxide); EDTA (ethylenediaminetetraacetic acid); Et (ethyl); EtOAc (ethyl acetate); Et₂O (diethyl ether); EtOH (ethanol); FBS (fetal bovine serum); FCC (flash column chromatography); FcεR1 (high-affinity IgE receptor); FLIPR (Fluorescence Imaging Plate Reader); g (gram(s)); h (hour(s)); GPCR (G protein-coupled receptor); HBSS (Hanks' Balanced Salt Solution); HCl (hydrochloric acid); HEPES (N-2-hydroxyethylpiperazine-N′-2-ethanesulfonic acid); Hex (hexanes); HLM (human liver microsome); HPLC (high performance liquid chromatography); hsMC (human skin mast cells); HTRF (Homogeneous Time Resolved Fluorescence); Hz (hertz); IgE (immunoglobulin E); IP1 (myo-Inositol 1 phosphate); IV (intravenous); J (coupling constant); JAK (janus kinase); K₂CO₃ (potassium carbonate); LCMS (liquid chromatography-mass spectrometry); m (multiplet); M (molar); MeCN (acetonitrile); mm (millimeter(s)); MS (Mass spectrometry); Me (methyl); MeOH (methanol); NADPH (nicotinamide adenine dinucleotide phosphate); NaI (sodium iodide); NaOtBu (sodium tert-butoxide); Na₂SO₃ (sodium sulfite); Na₂SO₄ (sodium sulfate); Na₂S₂O₃ (sodium thiosulfate); NFAT (Nuclear factor of activated T cells); PBS (phosphate-buffered saline); Ph (phenyl); prep. (preparative); PK (pharmacokinetic); PO (oral); rpm (revolutions per minute); r.t. (room temperature); RFU (Relative Fluorescence Unit); s (singlet or second(s)); sat. (saturated); SCF (stem cell factor); t (triplet or tertiary); TDI (time dependent inhibition); TEER (transepithelial electrical resistance); tert (tertiary); tt (triplet of triplets); t-Bu (tert-butyl); t-BuBrettPhos (2-(di-tert-butylphosphino)-2′,4′,6′-triisopropyl-3,6-dimethoxy-1,1′-biphenyl); tBuBrettPhos Pd G3 ([(2-di-tert-butylphosphino-3,6-dimethoxy-2′,4′,6′-triisopropyl-1,1′-biphenyl)-2-(2′-amino-1,1′-biphenyl)]palladium(II) methanesulfonate); TFA (trifluoroacetic acid); v/v (volume per volume); wt % (weight percent); w/v (weight in volume), μg (microgram(s)); μL (microliter(s)); μm (micrometer(s)).

Additional definitions are provided elsewhere in the present disclosure.

### II. Compounds

The present disclosure provides a compound having Formula (I):

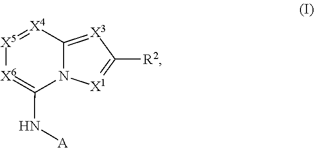

or a pharmaceutically acceptable salt thereof, wherein:
X¹ is N or CR¹;
X³ is N or CR³;
X⁴ is N or CR⁴;
X⁵ is N or CR⁵;
X⁶ is N or CR⁶;
R¹, R², R³, R⁴, R⁵, and R⁶ are each independently selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R¹, R², R³, R⁴, R⁵, and R⁶ are each optionally substituted with 1, 2, 3, or 4 substituents each independently selected from R^{10A}; and wherein the C₁₋₆ alkyl, C₂₋₆ alkenyl, and C₂₋₆ alkynyl forming R¹, R², R³, R⁴, R⁵, and R⁶ are each optionally substituted with 1, 2, 3, or 4 substituents each independently selected from R^{10B};
A is a group of the Formula (A-1) or (A-2):

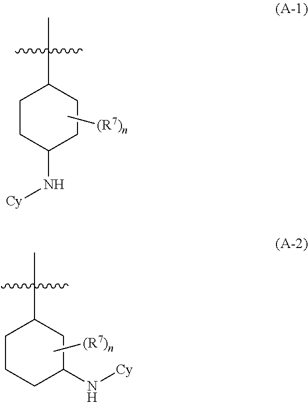

wherein [symbol] represents the point of attachment of A to the remainder of the molecule;
Cy is a ring selected from 5-10 membered heteroaryl and 5-10 membered heterocycloalkyl; and wherein the 5-10 membered heteroaryl and 5-10 membered heterocycloalkyl forming Cy are each optionally substituted with 1, 2, 3, 4, or 5 substituents independently selected from R^{Cy};
each R^{Cy} is independently selected from D, C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, N^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})N^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{Cy} are each optionally substituted with 1, 2, 3, or 4 substituents independently selected from R^{Cy1}; and wherein the C₁₋₆ alkyl, C₂₋₆ alkenyl, and C₂₋₆ alkynyl forming R^{Cy} are each optionally substituted with 1, 2, 3, or 4 substituents independently selected from R^{Cy2};
each R^{Cy1} is independently selected from C₁₋₆ alkyl, C₂₋₆ alkenyl, C₂₋₆ alkynyl, C₁₋₆ haloalkyl, and R^{Cy2}; wherein the C₁₋₆ alkyl, C₂₋₆ alkenyl, and C₂₋₆ alkynyl forming R^{Cy1} are each optionally substituted with 1, 2, 3, or 4 substituents independently selected from R^{Cy2};
each R^{Cy2} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a3}, SR^{a3}, C(O)R^{b3}, C(O)NR^{c3}R^{d3}, C(O)OR^{a3}, NR^{c3}R^{d3}, NR^{c3}C(O)R^{b3}, NR^{c3}C(O)OR^{a3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein the C₃₋₁₀ cycloalkyl, 4-10 membered heterocycloalkyl, C₆₋₁₀ to aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{Cy2} are each optionally substituted with 1, 2, 3, or 4 substituents independently selected from R^{g1};
R⁷ is selected from D, C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a4}, SR⁴, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NOR^{a4})R^{b4}, C(═NR^{e4})NR^{c4}R^{d4}, NR^{c4}C(═NR^{e4})N^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R⁷ are each optionally substituted with 1, 2, 3, or 4 substituents independently selected from R^{20A}; and wherein the C₁₋₆ alkyl, C₂₋₆ alkenyl, and C₂₋₆ alkynyl forming R⁷ are each optionally substituted with 1, 2, 3, or 4 substituents independently selected from R^{20B};
n is an integer from 0 to 8;
each R^{10A} is independently selected from C₁₋₆ alkyl, C₂₋₆ alkenyl, C₂₋₆ alkynyl, C₁₋₆ haloalkyl, and R^{10B}; wherein the C₁₋₆ alkyl, C₂₋₆ alkenyl, and C₂₋₆ alkynyl forming R^{10A} are each optionally substituted with 1, 2, 3, or 4 substituents independently selected from R^{10B};
each R^{10B} is independently selected from C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NOR^{a5})R^{b5}, C(═NR^{e5})NR^{c5}R^{d5}, NR^{c5}C(═NR^{e5})NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{10B} are each optionally substituted with 1, 2, 3, or 4 substituents independently selected from R¹¹;
each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a6}, SR^{a6}, C(O)R^{b6}, C(O)NR^{c6}R^{d6}, C(O)OR^{a6}, NR^{c6}R^{d6}, NR^{c6}C(O)R^{b6}, NR^{c6}C(O)OR^{a6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, and S(O)₂NR^{c6}R^{d6}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R¹¹ are each optionally substituted with 1, 2, 3, or 4 substituents independently selected from R^{g1}; and wherein the C₁₋₆ alkyl, C₂₋₆ alkenyl, and C₂₋₆ alkynyl forming R¹¹ are each optionally substituted with 1, 2, 3, or 4 substituents independently selected from R^{g2};
each R^{20A} is independently selected from C₁₋₆ alkyl, C₂₋₆ alkenyl, C₂₋₆ alkynyl, and C₁₋₆ haloalkyl; wherein the C₁₋₆ alkyl, C₂₋₆ alkenyl, and C₂₋₆ alkynyl forming R^{20A} are each optionally substituted with 1, 2, 3, or 4 substituents independently selected from R^{20B};
each R^{20B} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a7}, SR^{a7}, C(O)R^{b7}, C(O)NR^{c7}R^{d7}, C(O)OR^{a7}, NR^{c7}R^{d7}, NR^{c7}C(O)R^{b7}, NR^{c7}C(O)OR^{a7}, NR^{c7}S(O)R^{b7}, NR^{c7}S(O)₂R^{b7}, NR^{c7}S(O)₂NR^{c7}R^{d7}, S(O)R^{b7}, S(O)NR^{c7}R^{d7}, S(O)₂R^{b7}, and S(O)₂NR^{c7}R^{d7}; wherein the C₃₋₁₀ cycloalkyl, 4-10 membered heterocycloalkyl, C₆₋₁₀ to aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{20B} are each optionally substituted with 1, 2, 3, or 4 substituents independently selected from R^{g1};
each R^{a1}, R^{c1}, R^{d1}, R^{a2}, R^{c2}, R^{d2}, R^{a3}, R^{c3}, R^{d3}, R^{a4}, R^{c4}, R^{d4}, R^{a5}, R^{c5}, R^{d5}, R^{a6}, R^{c6}, R^{d6}, R^{a7}, R^{c7}, and R^{d7} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a1}, R^{c1}, R^{d1}, R^{a2}, R^{c2}, R^{d2}, R^{a3}, R^{c3}, R^{d3}, R^{a4}, R^{c4}, R^{d4}, R^{a5}, R^{c5}, R^{d5}, R^{a6}, R^{c6}, R^{d6}, R^{a7}, R^{c7}, and R^{d7} are each optionally substituted with 1, 2, 3, or 4 substituents independently selected from R^{g1}; and wherein the C₁₋₆ alkyl, C₂₋₆ alkenyl, and C₂₋₆ alkynyl forming R^{a1}, R^{c1}, R^{d1}, R^{a2}, R^{c2}, R^{d2}, R^{a3}, R^{c3}, R^{d3}, R^{a4}, R^{c4}, R^{d4}, R^{a5}, R^{c5}, R^{d5}, R^{a6}, R^{c6}, R^{d6}, R^{a7}, R^{c7}, and R^{d7} are each optionally substituted with 1, 2, 3, or 4 substituents independently selected from R^{g2};
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c6} and R^{d6} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c7} and R^{d7} attached to the same N atom, together with the N atom to which they are attached, form a 4-, 5-, 6- or 7-membered heterocycloalkyl group optionally substituted with 1, 2, 3, or 4 substituents independently selected from R^{g1};
each R^{b1}, R^{b2}, R^{b3}, R^{b4}, R^{b5}, R^{b6}, and R^{b7} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b1}, R^{b2}, R^{b3}, R^{b4}, R^{b5}, R^{b6}, and R^{b7} are each optionally substituted with 1, 2, 3, or 4 substituents independently selected from R^{g1}; and wherein the C₁₋₆ alkyl, C₂₋₆ alkenyl, and C₂₋₆ alkynyl forming R^{b1}, R^{b2}, R^{b3}, R^{b4}, R^{b5}, R^{b6}, and R^{b7} are each optionally substituted with 1, 2, 3, or 4 substituents independently selected from R^{g2};
each R^{e1}, R^{e2}, R^{e4}, and R^{e5} is independently selected from H, CN, C₁₋₆alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆alkyl)aminosulfonyl;
each R^{g1} is independently selected from C₁₋₆ alkyl, C₂₋₆ alkenyl, C₂₋₆ alkynyl, and R^{g2}, wherein the C₁₋₆ alkyl, C₂₋₆ alkenyl, and C₂₋₆ alkynyl are each optionally substituted with 1, 2, or 3 substituents independently selected from R^{g2};
each R^{g2} is independently selected from C₃₋₁₀ cycloalkyl, 4-10 membered heterocycloalkyl, C₆₋₁₀ aryl, 5-10 membered heteroaryl, halo, CN, OR^{a8}, SR^{a8}, C(O)R^{b8}, C(O)NR^{c8}R^{d8}, C(O)OR^{a8}, NR^{c8}R^{d8}, NR^{c8}C(O)R^{b8}, NR^{c8}C(O)OR^{a8}, NR^{c8}S(O)R^{b8}, NR^{c8}S(O)₂R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8}; wherein the C₃₋₁₀ cycloalkyl, 4-10 membered heterocycloalkyl, C₆₋₁₀ aryl, and 5-10 membered heteroaryl forming R^{g2} are each optionally substituted with 1, 2, 3, or 4 substituents independently selected from R^{h1};
each R^{a8}, R^{c8}, and R^{d8} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a8}, R^{c8}, and R^{d8} are each optionally substituted with 1, 2, 3, or 4 substituents independently selected from R^{h1}; and wherein the C₁₋₆ alkyl, C₂₋₆ alkenyl, and C₂₋₆ alkynyl forming R^{a8}, R^{c8}, and R^{d8} are each optionally substituted with 1, 2, 3, or 4 substituents independently selected from R^{h2};
each R^{b8} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b8} are each optionally substituted with 1, 2, 3, or 4 substituents independently selected from R^{h1}; and wherein the C₁₋₆ alkyl, C₂₋₆ alkenyl, and C₂₋₆ alkynyl forming R^{b11} are each optionally substituted with 1, 2, 3, or 4 substituents independently selected from R^{h2};
each R^{h1} is independently selected from C₁₋₆ alkyl, C₂₋₆ alkenyl, C₂₋₆ alkynyl, and R^{g2}, wherein the C₁₋₆ alkyl, C₂₋₆ alkenyl, and C₂₋₆ alkynyl are each optionally substituted with 1, 2, or 3 substituents independently selected from R^{h2};
each R^{h2} is independently selected from D, OH, NO₂, CN, halo, C₃₄ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino;
wherein at each occurrence, a heterocycloalkyl group has at least one ring-forming carbon atom and 1, 2, 3, or 4 ring-forming heteroatoms independently selected from N, O, and S; the N and S ring-forming heteroatoms of the heterocycloalkyl group are optionally oxidized; and a ring-forming carbon atom of the heterocycloalkyl group is optionally substituted by oxo to form a carbonyl group; and
at each occurrence, a heteroaryl group has at least one ring-forming carbon atom and 1, 2, 3, or 4 ring-forming heteroatoms independently selected from N, O, and S; the N and S ring-forming heteroatoms of the heteroaryl group are optionally oxidized; and a ring-forming carbon atom of the heteroaryl group is optionally substituted by oxo to form a carbonyl group.

In some embodiments, the compound is not one of the following compounds:
N¹-(5-Methyl[1,2,4]triazolo[1,5-a]pyrimidin-7-yl)-N⁴-2-pyridinyl-1,4-cyclohexanediamine;
N¹,N⁴-Bis(5,6-dimethyl[1,2,4]triazolo[1,5-a]pyrimidin-7-yl)-1,4-cyclohexanediamine;
N¹-[3-(2,6-Dichloro-4-methoxyphenyl)-2,5-dimethylpyrazolo[1,5-a]pyrimidin-7-yl]-N⁴-(tetrahydro-2H-pyran-4-yl)-1,4-cyclohexanediamine;
N¹-[3-(2,6-Dichloro-4-methoxyphenyl)-2,5-dimethylpyrazolo[1,5-a]pyrimidin-7-yl]-N³-(tetrahydro-2H-pyran-4-yl)-1,3-cyclohexanediamine;
N¹-[3-(2,6-Dichloro-4-methoxyphenyl)-2,5-dimethylpyrazolo[1,5-a]pyrimidin-7-yl]-N³-[1-(phenylmethyl)-4-piperidinyl]-1,3-cyclohexanediamine.

In some embodiments, the compound has Formula (II-A), (II-B), (II-C), (II-D), (II-E), or (II-F):

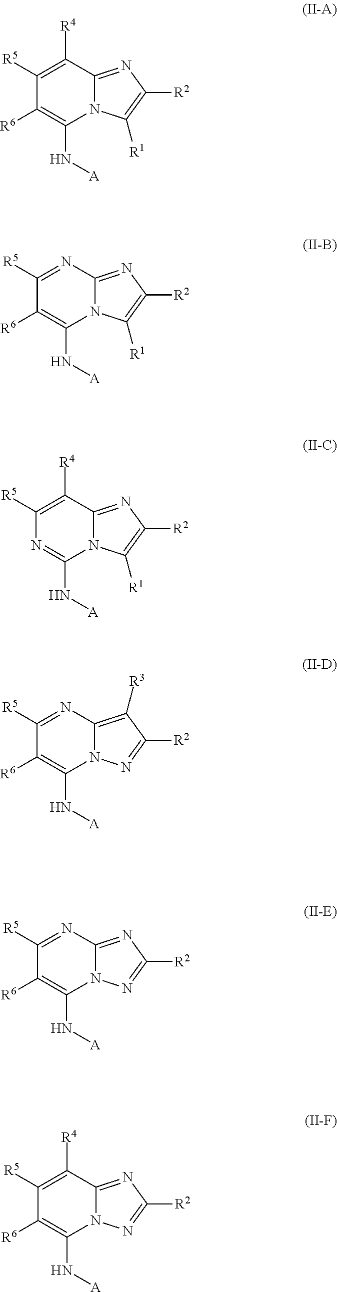

or a pharmaceutically acceptable salt thereof, wherein R¹, R², R³, R⁴, R⁵, R⁶, and A are as defined herein.

In some embodiments, the compound is other than a compound of Formula (II-A); the compound is other than a compound of Formula (II-B); the compound is other than a compound of Formula (II-C); the compound is other than a compound of Formula (II-D); the compound is other than a compound of Formula (II-E); and/or the compound is other than a compound of Formula (II-F).

In some embodiments, A is a group of the Formula (A-1).

In some embodiments, A is a group of the Formula (A-3):

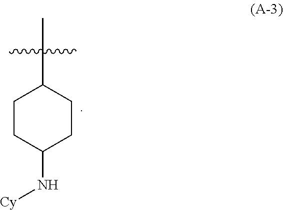

In some embodiments, A is a group of the Formula (A-4), (A-5), (A-6), or (A-7):

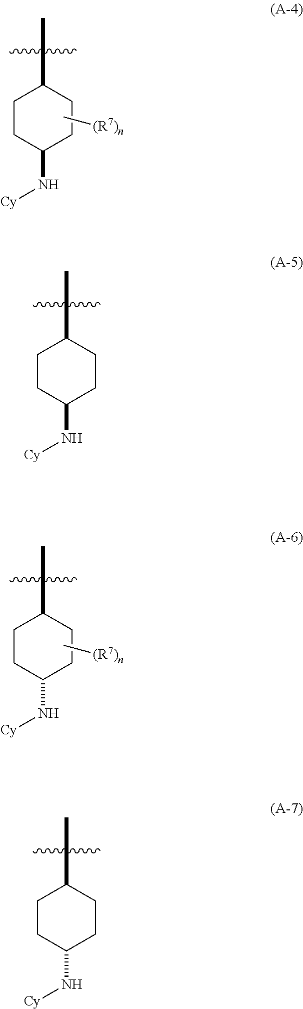

wherein the bold and dashed lines in the Formulae (A-4), (A-5), (A-6), and (A-7) indicate relative (i.e., cis or trans) stereochemistry.

In some embodiments, A is a group of the Formula (A-2).

In some embodiments, A is a group of the Formula (A-8):

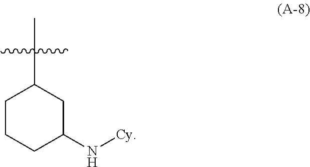

In some embodiments, A is a group of the Formula (A-9) or (A-10):

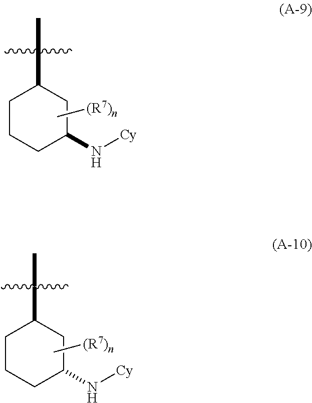

wherein the bold and dashed lines in the Formulae (A-7) and (A-8) indicate relative (i.e., cis or trans) stereochemistry.

In some embodiments, A is a group of the Formula (A-11), (A-12), (A-13) or (A-14):

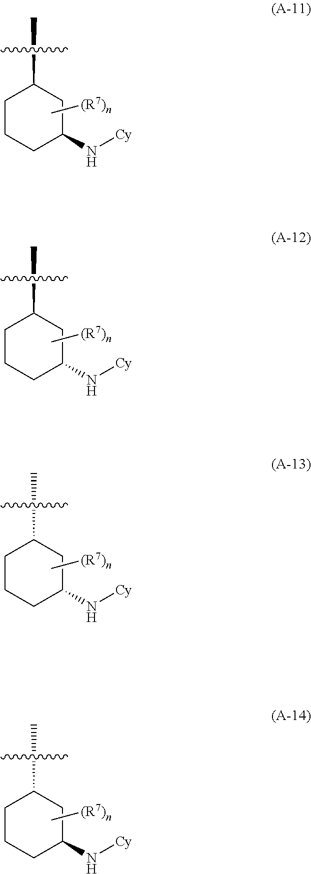

wherein the wedged lines in the Formulae (A-11), (A-12), (A-13) and (A-14) indicate absolute (i.e., (R) or (S)) stereochemistry.

In some embodiments, A is a group of the Formula (A-15) or (A-16):

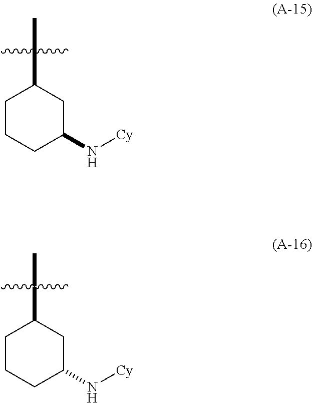

wherein the bold and dashed lines in the Formulae (A-15) and (A-16) indicate relative (i.e., cis or trans) stereochemistry.

In some embodiments, A is a group of the Formula (A-17), (A-18), (A-19) or (A-20):

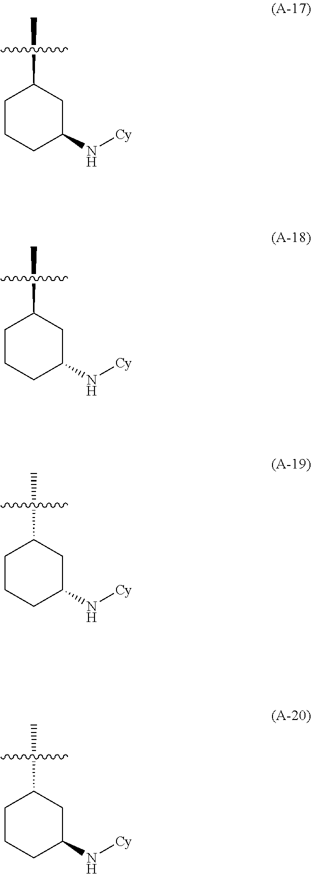

wherein the wedged lines in the Formulae (A-17), (A-18), (A-19) and (A-20) indicate absolute (i.e., (R) or (S)) stereochemistry.

In some embodiments, X¹ is N. In some embodiments, X¹ is CR¹.

In some embodiments, R¹ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═OR^{a1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}NR^{d1}, P(O)(NH₂)R^{b1}, or P(O)(NH₂)OR^{a1}; wherein the C₁₋₆ alkyl, C₂₋₆ alkenyl, and C₂₋₆ alkynyl forming R² are each optionally substituted with 1, 2, 3, or 4 substituents each independently selected from R^{10B}.

In some embodiments, R¹ is selected from H, D, C₁₋₆ alkyl, C₁₋₆ haloalkyl, halo, CN, OR^{a1}; wherein the C₁₋₆ alkyl forming R¹ is optionally substituted with 1, 2, 3, or 4 substituents each independently selected from R^{10B}. In some embodiments, R¹ is selected from H, D, C₁₋₆ alkyl (such as methyl or ethyl), C₁₋₆ haloalkyl (such as CF₃, CHF₂, CF₂CF₃), CN, or halo (such as F, Cl, or Br). In some embodiments, R¹ is H.

In some embodiments, X² is N. In some embodiments, X² is CR².

In some embodiments, R² is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}NR^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₁₋₆ alkyl, C₂₋₆ alkenyl, and C₂₋₆ alkynyl forming R² are each optionally substituted with 1, 2, 3, or 4 substituents each independently selected from R^{10B}.

In some embodiments, R² is H, D, C₁₋₆ alkyl (such as methyl or ethyl), C₁₋₆ haloalkyl (such as CF₃, CHF₂, CF₂CF₃), CN, or halo (such as F, Cl, or Br). In some embodiments, R² is H. In some embodiments, R² is C₁₋₆ alkyl, C₁₋₆ haloalkyl, or halo. In some embodiments, R² is CF₃, CH₃, or Br.

In some embodiments, X³ is N. In some embodiments, X³ is CR³.

In some embodiments, R³ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₁₋₆ alkyl, C₂₋₆ alkenyl, and C₂₋₆ alkynyl forming R³ are each optionally substituted with 1, 2, 3, or 4 substituents each independently selected from R^{10B}.

In some embodiments, R³ is selected from H, D, C₁₋₆ alkyl, C₁₋₆ haloalkyl, halo, CN, OR^{a1}; wherein the C₁₋₆ alkyl forming R³ is optionally substituted with 1, 2, 3, or 4 substituents each independently selected from R^{10B}. In some embodiments, R³ is H, D, C₁₋₆ alkyl (such as methyl or ethyl), C₁₋₆ haloalkyl (such as CF₃, CHF₂, CF₂CF₃), CN, or halo (such as F, Cl, or Br). In some embodiments, R³ is H.

In some embodiments, X⁴ is N. In some embodiments, X⁴ is CR⁴.

In some embodiments, R⁴ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}NR^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{c1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₁₋₆ alkyl, C₂₋₆ alkenyl, and C₂₋₆ alkynyl forming R⁴ are each optionally substituted with 1, 2, 3, or 4 substituents each independently selected from R^{10B}.

In some embodiments, R⁴ is selected from H, D, C₁₋₆ alkyl, C₁₋₆ haloalkyl, halo, CN, OR^{a1}; wherein the C₁₋₆ alkyl forming R⁴ is optionally substituted with 1, 2, 3, or 4 substituents each independently selected from R^{10B}. In some embodiments, R⁴ is H, D, C₁₋₆ alkyl (such as methyl or ethyl), C₁₋₆ haloalkyl (such as CF₃, CHF₂, CF₂CF₃), CN, or halo (such as F, Cl, or Br). In some embodiments, R⁴ is H.

In some embodiments, X⁵ is N. In some embodiments, X⁵ is CR⁵.

In some embodiments, R⁵ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{c1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₁₋₆ alkyl, C₂₋₆ alkenyl, and C₂₋₆ alkynyl forming R⁵ are each optionally substituted with 1, 2, 3, or 4 substituents each independently selected from R^{10B}.

In some embodiments, R⁵ is H, D, C₁₋₆ alkyl (such as methyl or ethyl), C₁₋₆ haloalkyl (such as CF₃, CHF₂, CF₂CF₃), CN, or halo (such as F, Cl, or Br). In some embodiments, R⁵ is H. In some embodiments, R⁵ is H or C₁₋₆ haloalkyl. In some embodiments, In some embodiments, R⁵ is C₁₋₆ haloalkyl. In some embodiments, In some embodiments, R⁵ is H or CF₃. In some embodiments, R⁵ is CF₃.

In some embodiments, X⁶ is N. In some embodiments, X⁶ is CR⁶.

In some embodiments, R⁶ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₁₋₆ alkyl, C₂₋₆ alkenyl, and C₂₋₆ alkynyl forming R⁶ are each optionally substituted with 1, 2, 3, or 4 substituents each independently selected from R^{10B}.

In some embodiments, R⁶ is selected from H, D, C₁₋₆ alkyl, C₁₋₆ haloalkyl, halo, CN, OR^{a1}; wherein the C₁₋₆ alkyl forming R⁶ is optionally substituted with 1, 2, 3, or 4 substituents each independently selected from R^{10B}. In some embodiments, R⁶ is H, D, C₁₋₆ alkyl (such as methyl or ethyl), C₁₋₆ haloalkyl (such as CF₃, CHF₂, CF₂CF₃), CN, or halo (such as F, Cl, or Br). In some embodiments, R⁶ is H.

In some embodiments, R⁷ is selected from D, C₁₋₆ alkyl, C₂₋₆ alkenyl, C₂₋₆ alkynyl, C₁₋₆ haloalkyl, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NOR^{a4})R^{b4}, C(═NR^{e4})NR^{c4}R^{d4}, NR^{c4}C(═NR^{e4})NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein the C₁₋₆ alkyl, C₂₋₆ alkenyl, and C₂₋₆ alkynyl forming R⁷ are each optionally substituted with 1, 2, 3, or 4 substituents each independently selected from R^{20B}.

In some embodiments, R⁷ is D, C₁₋₆ alkyl (such as methyl or ethyl), C₁₋₆ haloalkyl (such as CF₃, CHF₂, CF₂CF₃), CN, or halo (such as F, Cl, or Br).

In some embodiments, Cy is optionally substituted with 1, 2, 3, 4, 5 or 6 substituents independently selected from R^{Cy}. In some embodiments, Cy is optionally substituted with 1, 2, 3, 4, or 5 substituents independently selected from R^{Cy}. In some embodiments, Cy is optionally substituted with 1, 2, 3, or 4 substituents independently selected from R^{Cy}. In some embodiments, Cy is optionally substituted with 1, 2, or 3 substituents independently selected from R^{Cy}. In some embodiments, Cy is optionally substituted with 1 or 2 substituents independently selected from R^{Cy}. In some embodiments, Cy is optionally substituted with 1 substituent selected from R^{Cy}. In some embodiments, Cy is unsubstituted.

In some embodiments, Cy is 5-10 membered heteroaryl optionally substituted with 1, 2, 3, 4, 5 or 6 substituents independently selected from R^{Cy}. In some embodiments, Cy is 5-10 membered heteroaryl optionally substituted with 1, 2, 3, 4, or 5 substituents independently selected from R^{Cy}. In some embodiments, Cy is 5-10 membered heteroaryl optionally substituted with 1, 2, 3, or 4 substituents independently selected from R^{Cy}. In some embodiments, Cy is 5-10 membered heteroaryl optionally substituted with 1, 2, or 3 substituents independently selected from R^{Cy}. In some embodiments, Cy is 5-10 membered heteroaryl optionally substituted with 1 or 2 substituents independently selected from R^{Cy}. In some embodiments, Cy is 5-10 membered heteroaryl optionally substituted with 1 substituent selected from R^{Cy}.

In some embodiments, Cy is unsubstituted 5-10 membered heteroaryl. In some embodiments, Cy is unsubstituted or substituted 3H-imidazo[4,5-c]pyridin-4-yl, 1,6-naphthyridin-5-yl, imidazo[1,5-a]pyrazin-8-yl, pyrazolo[1,5-a]pyrazin-4-yl, [1,2,4]triazolo[4,3-a]pyridin-3-yl, or imidazo[1,2-c]pyrimidin-5-yl.

In some embodiments, Cy is unsubstituted 5-10 membered heteroaryl. In some embodiments, Cy is unsubstituted or substituted 3H-imidazo[4,5-c]pyridinyl, 1,6-naphthyridinyl, imidazo[1,5-a]pyrazinyl, pyrazolo[1,5-a]pyrazinyl, [1,2,4]triazolo[4,3-a]pyridinyl, or imidazo[1,2-c]pyrimidinyl.

In some embodiments, each R^{Cy} is independently selected from C₁₋₆ alkyl, C₂₋₆ alkenyl, C₂₋₆ alkynyl, C₁₋₆ haloalkyl, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})R^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein the C₁₋₆ alkyl, C₂₋₆ alkenyl, and C₂₋₆ alkynyl forming R^{Cy} are each optionally substituted with 1, 2, 3, or 4 substituents independently selected from R^{Cy2}.

In some embodiments, each R^{Cy} is selected from C₁₋₆ alkyl (such as methyl or ethyl), C₁₋₆ haloalkyl (such as CF₃, CHF₂, CF₂CF₃), halo (such as F, Cl, or Br), and OR^{a2} (such as methoxy or ethoxy). In some embodiments, each R^{Cy} is selected from C₁₋₆ alkyl. In some embodiments, R^{Cy} is CH₃.

In some embodiments, n is 0. In some embodiments, n is an integer from 1 to 8. In some embodiments, n is an integer from 1 to 4. In some embodiments, n is 1 or 2. In some embodiments, n is 0 or 1. In some embodiments, n is 0, 1, or 2.

Also provided herein is a compound of Formula (I), wherein:
X¹ is N or CR¹;
X³ is N or CR³;
X⁴ is N or CR⁴;
X⁵ is N or CR⁵;
X⁶ is N or CR⁶;
R¹, R², R³, R⁴, R⁵, and R⁶ are each independently selected from H, D, C₁₋₆ alkyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, NR^{c1}R^{d1}, and S(O)₂R^{b1};
A is a group of the Formula (A-1) or (A-2):

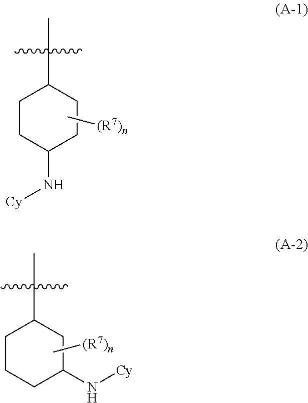

wherein [symbol] represents the point of attachment of A to the remainder of the molecule;
Cy is a ring selected from 5-10 membered heteroaryl optionally substituted with 1, 2, 3, 4, or 5 substituents independently selected from R^{Cy};
each R^{Cy} is independently selected from C₁₋₆ alkyl, C₃₋₁₀ cycloalkyl, 4-12 membered heterocycloalkyl, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R⁷ is selected from D, C₁₋₆ alkyl, C₁₋₆ haloalkyl, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NOR^{a4})R^{b4}, C(═NR^{e4})NR^{c4}R^{d4}, NR^{c4}C(═NR⁴)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};
n is an integer from 0 to 8;
each R^{a1}, R^{c1}, R^{d1}, R^{a2}, R^{c2}, R^{d2}, R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
each R^{b1}, R^{b2}, and R^{b4} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
each R^{e2} and R^{e4} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{g1} is independently selected from C₁₋₆ alkyl, C₂₋₆ alkenyl, C₂₋₆ alkynyl, and R^{g2}, wherein the C₁₋₆ alkyl, C₂₋₆ alkenyl, and C₂₋₆ alkynyl are each optionally substituted with 1, 2, or 3 substituents independently selected from R^{g2};
each R^{g2} is independently selected from C₃₋₁₀ cycloalkyl, 4-10 membered heterocycloalkyl, C₆₋₁₀ aryl, 5-10 membered heteroaryl, halo, CN, OR^{a8}, SR^{a8}s, C(O)R^{b8}, C(O)NR^{c8}R^{d8}, C(O)OR^{a8}, NR^{c8}R^{d8}, NR^{c8}C(O)R^{b8}, NR^{c8}C(O)OR^{a8}, NR^{c8}S(O)R^{b8}, NR^{c8}S(O)₂R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8};
each R^{a8}, R^{c8}, and R^{d8} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
each R^{b8} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
wherein at each occurrence, a heterocycloalkyl group has at least one ring-forming carbon atom and 1, 2, 3, or 4 ring-forming heteroatoms independently selected from N, O, and S; the N and S ring-forming heteroatoms of the heterocycloalkyl group are optionally oxidized; and a ring-forming carbon atom of the heterocycloalkyl group is optionally substituted by oxo to form a carbonyl group; and
at each occurrence, a heteroaryl group has at least one ring-forming carbon atom and 1, 2, 3, or 4 ring-forming heteroatoms independently selected from N, O, and S; the N and S ring-forming heteroatoms of the heteroaryl group are optionally oxidized; and a ring-forming carbon atom of the heteroaryl group is optionally substituted by oxo to form a carbonyl group;
with the proviso that the compound is not one of the following compounds:
N¹-(5-Methyl[1,2,4]triazolo[1,5-a]pyrimidin-7-yl)-N⁴-2-pyridinyl-1,4-cyclohexanediamine;
N¹,N⁴-Bis(5,6-dimethyl[1,2,4]triazolo[1,5-a]pyrimidin-7-yl)-1,4-cyclohexanediamine;
N¹-[3-(2,6-Dichloro-4-methoxyphenyl)-2,5-dimethylpyrazolo[1,5-a]pyrimidin-7-yl]-N⁴-(tetrahydro-2H-pyran-4-yl)-1,4-cyclohexanediamine;
N¹-[3-(2,6-Dichloro-4-methoxyphenyl)-2,5-dimethylpyrazolo[1,5-a]pyrimidin-7-yl]-N³-(tetrahydro-2H-pyran-4-yl)-1,3-cyclohexanediamine;
N¹-[3-(2,6-Dichloro-4-methoxyphenyl)-2,5-dimethylpyrazolo[1,5-a]pyrimidin-7-yl]-N³-[1-(phenylmethyl)-4-piperidinyl]-1,3-cyclohexanediamine.

Also provided herein is a compound of Formula (IIA):

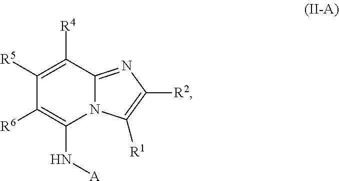

wherein:
R¹, R², R⁴, R⁵, and R⁶ are each independently selected from H, D, C₁₋₆ alkyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, NR^{c1}R^{d1}, and S(O)₂R^{b1};
A is a group of the Formula (A-4):

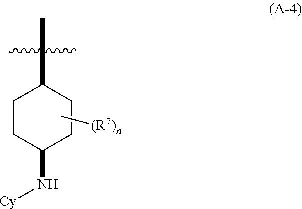

wherein [symbol] represents the point of attachment of A to the remainder of the molecule;
Cy is a ring selected from 5-10 membered heteroaryl optionally substituted with 1, 2, 3, 4, or 5 substituents independently selected from R^{Cy};
each R^{Cy} is independently selected from C₁₋₆ alkyl, C₃₋₁₀ cycloalkyl, 4-12 membered heterocycloalkyl, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})R^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R⁷ is selected from D, C₁₋₆ alkyl, C₁₋₆ haloalkyl, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NOR^{a4})R^{b4}, C(═NR^{e4})NR^{c4}R^{d4}, NR^{c4}C(═NR⁴)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};
n is an integer from 0 to 8;
each R^{a1}, R^{c1}, R^{d1}, R^{a2}, R^{c2}, R^{d2}, R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
each R^{b1}, R^{b2}, and R^{b4} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
each R^{e2} and R^{e4} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{g1} is independently selected from C₁₋₆ alkyl, C₂₋₆ alkenyl, C₂₋₆ alkynyl, and R^{g2}, wherein the C₁₋₆ alkyl, C₂₋₆ alkenyl, and C₂₋₆ alkynyl are each optionally substituted with 1, 2, or 3 substituents independently selected from R^{g2};
each R^{g2} is independently selected from C₃₋₁₀ cycloalkyl, 4-10 membered heterocycloalkyl, C₆₋₁₀ aryl, 5-10 membered heteroaryl, halo, CN, OR^{a8}, SR^{a8}, C(O)R^{b8}, C(O)NR^{c8}R^{d8}, C(O)OR^{a8}, NR^{c8}R^{d8}, NR^{c8}C(O)R^{b8}, NR^{c8}C(O)OR^{a8}, NR^{c8}S(O)R^{b8}, NR^{c8}S(O)₂R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8};
each R^{a8}, R^{c8}, and R^{d8} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
each R^{b8} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
wherein at each occurrence, a heterocycloalkyl group has at least one ring-forming carbon atom and 1, 2, 3, or 4 ring-forming heteroatoms independently selected from N, O, and S; the N and S ring-forming heteroatoms of the heterocycloalkyl group are optionally oxidized; and a ring-forming carbon atom of the heterocycloalkyl group is optionally substituted by oxo to form a carbonyl group; and
at each occurrence, a heteroaryl group has at least one ring-forming carbon atom and 1, 2, 3, or 4 ring-forming heteroatoms independently selected from N, O, and S; the N and S ring-forming heteroatoms of the heteroaryl group are optionally oxidized; and a ring-forming carbon atom of the heteroaryl group is optionally substituted by oxo to form a carbonyl group.

Also provided herein is a compound of Formula (II-A):

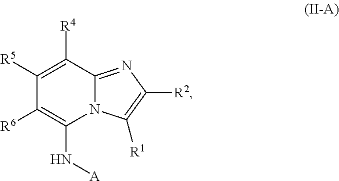

wherein:
R¹, R², R⁴, R⁵, and R⁶ are each independently selected from H, D, C₁₋₆ alkyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, NR^{c1}R^{d1}, and S(O)₂R^{b1};
A is a group of the Formula (A-9):

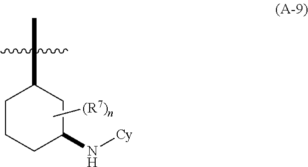

wherein [symbol] represents the point of attachment of A to the remainder of the molecule;
Cy is a ring selected from 5-10 membered heteroaryl optionally substituted with 1, 2, 3, 4, or 5 substituents independently selected from R^{Cy};
each R^{Cy} is independently selected from C₁₋₆ alkyl, C₃₋₁₀ cycloalkyl, 4-12 membered heterocycloalkyl, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R⁷ is selected from D, C₁₋₆ alkyl, C₁₋₆ haloalkyl, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NOR^{a4})R^{b4}, C(═NR^{e4})NR^{c4}R^{d4}, NR^{c4}C(═NR^{e4})NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};
n is an integer from 0 to 8;
each R^{a1}, R^{c1}, R^{d1}, R^{a2}, R^{c2}, R^{d2}, R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
each R^{b1}, R^{b2}, and R^{b4} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
each R^{e2} and R^{e4} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{g1} is independently selected from C₁₋₆ alkyl, C₂₋₆ alkenyl, C₂₋₆ alkynyl, and R^{g2}, wherein the C₁₋₆ alkyl, C₂₋₆ alkenyl, and C₂₋₆ alkynyl are each optionally substituted with 1, 2, or 3 substituents independently selected from R^{g2};
each R^{g2} is independently selected from C₃₋₁₀ cycloalkyl, 4-10 membered heterocycloalkyl, C₆₋₁₀ aryl, 5-10 membered heteroaryl, halo, CN, OR^{a8}, SR^{a8}, C(O)R^{b8}, C(O)NR^{c8}R^{d8}, C(O)OR^{a8}, NR^{c8}R^{d8}, NR^{c8}C(O)R^{b8}, NR^{c8}C(O)OR^{a8}, NR^{c8}S(O)R^{b8}, NR^{c8}S(O)₂R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8};
each R^{a8}, R^{c8}, and R^{d8} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
each R^{b8} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
wherein at each occurrence, a heterocycloalkyl group has at least one ring-forming carbon atom and 1, 2, 3, or 4 ring-forming heteroatoms independently selected from N, O, and S; the N and S ring-forming heteroatoms of the heterocycloalkyl group are optionally oxidized; and a ring-forming carbon atom of the heterocycloalkyl group is optionally substituted by oxo to form a carbonyl group; and
at each occurrence, a heteroaryl group has at least one ring-forming carbon atom and 1, 2, 3, or 4 ring-forming heteroatoms independently selected from N, O, and S; the N and S ring-forming heteroatoms of the heteroaryl group are optionally oxidized; and a ring-forming carbon atom of the heteroaryl group is optionally substituted by oxo to form a carbonyl group.

Also provided herein is a compound of Formula (II-D):

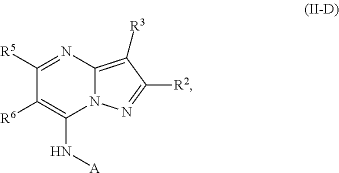

wherein:
R², R³, R⁵, and R⁶ are each independently selected from H, D, C₁₋₆ alkyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, NR^{c1}R^{d1}, and S(O)₂R^{b1};
A is a group of the Formula (A-9):

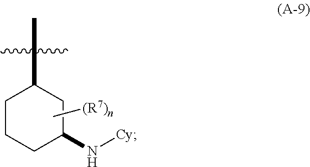

wherein [symbol] represents the point of attachment of A to the remainder of the molecule;
Cy is a ring selected from 5-10 membered heteroaryl optionally substituted with 1, 2, 3, 4, or 5 substituents independently selected from R^{Cy};
each R^{Cy} is independently selected from C₁₋₆ alkyl, C₃₋₁₀ cycloalkyl, 4-12 membered heterocycloalkyl, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})R^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R⁷ is selected from D, C₁₋₆ alkyl, C₁₋₆ haloalkyl, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NOR^{a4})R^{b4}, C(═NR^{e4})NR^{c4}R^{d4}, NR^{c4}C(═NR^{e4})NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};
n is an integer from 0 to 8;
each R^{a1}, R^{c1}, R^{d1}, R^{a2}, R^{c2}, R^{d2}, R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
each R^{b1}, R^{b2}, and R^{b4} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
each R^{e2} and R^{e4} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{g1} is independently selected from C₁₋₆ alkyl, C₂₋₆ alkenyl, C₂₋₆ alkynyl, and R^{g2}, wherein the C₁₋₆ alkyl, C₂₋₆ alkenyl, and C₂₋₆ alkynyl are each optionally substituted with 1, 2, or 3 substituents independently selected from R^{g2};
each R^{g2} is independently selected from C₃₋₁₀ cycloalkyl, 4-10 membered heterocycloalkyl, C₆₋₁₀ aryl, 5-10 membered heteroaryl, halo, CN, OR^{a8}, SR^{a8}, C(O)R^{b8}, C(O)NR^{c8}R^{d8}, C(O)OR^{a8}, NR^{c8}R^{d8}, NR^{c8}C(O)R^{b8}, NR^{c8}C(O)OR^{a8}, NR^{c8}S(O)R^{b8}, NR^{c8}S(O)₂R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8};
each R^{a8}, R^{c8}, and R^{d8} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
each R^{b8} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
wherein at each occurrence, a heterocycloalkyl group has at least one ring-forming carbon atom and 1, 2, 3, or 4 ring-forming heteroatoms independently selected from N, O, and S; the N and S ring-forming heteroatoms of the heterocycloalkyl group are optionally oxidized; and a ring-forming carbon atom of the heterocycloalkyl group is optionally substituted by oxo to form a carbonyl group; and
at each occurrence, a heteroaryl group has at least one ring-forming carbon atom and 1, 2, 3, or 4 ring-forming heteroatoms independently selected from N, O, and S; the N and S ring-forming heteroatoms of the heteroaryl group are optionally oxidized; and a ring-forming carbon atom of the heteroaryl group is optionally substituted by oxo to form a carbonyl group;
with the proviso that the compound is not one of the following compounds:
N¹-[3-(2,6-Dichloro-4-methoxyphenyl)-2,5-dimethylpyrazolo[1,5-a]pyrimidin-7-yl]-N³-(tetrahydro-2H-pyran-4-yl)-1,3-cyclohexanediamine; and
N¹-[3-(2,6-Dichloro-4-methoxyphenyl)-2,5-dimethylpyrazolo[1,5-a]pyrimidin-7-yl]-N³-[1-(phenylmethyl)-4-piperidinyl]-1,3-cyclohexanediamine.

In some embodiments, the compound of Formula (I) is selected from:
(1R,3S)—N¹-(3-methyl-3H-imidazo[4,5-c]pyridin-4-yl)-N³-(2-(trifluoromethyl)imidazo[1,2-a]pyridin-5-yl)cyclohexane-1,3-diamine;
(1s,4s)-N¹-(3-methyl-3H-imidazo[4,5-c]pyridin-4-yl)-N⁴-(2-(trifluoromethyl)imidazo[1,2-a]pyridin-5-yl)cyclohexane-1,4-diamine;
(1R,3S)—N¹-(1,6-naphthyridin-5-yl)-N³-(2-(trifluoromethyl)imidazo[1,2-a]pyridin-5-yl)cyclohexane-1,3-diamine;
(1R,3S)—N¹-(imidazo[1,5-a]pyrazin-8-yl)-N³-(2-(trifluoromethyl)imidazo[1,2-a]pyridin-5-yl)cyclohexane-1,3-diamine;
(1s,4s)-N¹-(imidazo[1,5-a]pyrazin-8-yl)-N⁴-(2-(trifluoromethyl)imidazo[1,2-a]pyridin-5-yl)cyclohexane-1,4-diamine;
(1R,3S)—N¹-(3-methyl-3H-imidazo[4,5-c]pyridin-4-yl)-N³-(2-methyl-5-(trifluoromethyl)pyrazolo[1,5-a]pyrimidin-7-yl)cyclohexane-1,3-diamine;
(1S,3R)—N¹-(2-methyl-5-(trifluoromethyl)pyrazolo[1,5-a]pyrimidin-7-yl)-N³-(1,6-naphthyridin-5-yl)cyclohexane-1,3-diamine;
(1S,3R)—N¹-(2-bromo-5-(trifluoromethyl)pyrazolo[1,5-a]pyrimidin-7-yl)-N³-(pyrazolo[1,5-a]pyrazin-4-yl)cyclohexane-1,3-diamine;
(1S,3R)—N¹-(2-bromo-5-(trifluoromethyl)pyrazolo[1,5-a]pyrimidin-7-yl)-N³-(1,6-naphthyridin-5-yl)cyclohexane-1,3-diamine;
(1R,3S)—N¹—([1,2,4]triazolo[4,3-a]pyridin-3-yl)-N³-(2-bromo-5-(trifluoromethyl)pyrazolo[1,5-a]pyrimidin-7-yl)cyclohexane-1,3-diamine;
(1S,3R)—N¹-(2,5-bis(trifluoromethyl)pyrazolo[1,5-a]pyrimidin-7-yl)-N³-(1,6-naphthyridin-5-yl)cyclohexane-1,3-diamine;
(1S,3R)—N¹-(2-bromo-5-(trifluoromethyl)pyrazolo[1,5-a]pyrimidin-7-yl)-N³-(imidazo[1,5-a]pyrazin-8-yl)cyclohexane-1,3-diamine;
(1S,3R)—N¹-(2-bromo-5-(trifluoromethyl)pyrazolo[1,5-a]pyrimidin-7-yl)-N³-(3-methyl-3H-imidazo[4,5-c]pyridin-4-yl)cyclohexane-1,3-diamine;
(1S,3R)—N¹-(2,5-bis(trifluoromethyl)pyrazolo[1,5-a]pyrimidin-7-yl)-N³-(imidazo[1,5-a]pyrazin-8-yl)cyclohexane-1,3-diamine;
(1S,3R)—N¹-(2,5-bis(trifluoromethyl)pyrazolo[1,5-a]pyrimidin-7-yl)-N³-(3-methyl-3H-imidazo[4,5-c]pyridin-4-yl)cyclohexane-1,3-diamine;
(1S,3R)—N¹-(2-bromo-5-(trifluoromethyl)pyrazolo[1,5-a]pyrimidin-7-yl)-N³-(imidazo[1,2-c]pyrimidin-5-yl)cyclohexane-1,3-diamine;
(1S,3R)—N¹-(2,5-bis(trifluoromethyl)pyrazolo[1,5-a]pyrimidin-7-yl)-N³-(imidazo[1,2-c]pyrimidin-5-yl)cyclohexane-1,3-diamine; and
(1R,3S)—N¹—([1,2,4]triazolo[4,3-a]pyridin-3-yl)-N³-(2,5-bis(trifluoromethyl)pyrazolo[1,5-a]pyrimidin-7-yl)cyclohexane-1,3-diamine;

or a pharmaceutically acceptable salt of any of the aforementioned.

In other embodiments, the compound of Formula (I) is in the form of a pharmaceutically acceptable salt. In other embodiments, the compound of Formula (I) is in the form of a free base or free acid, or other than in the form of a salt.

In another aspect, provided herein is a pharmaceutical composition comprising a compound of Formula (I), or any of the embodiments thereof, or a pharmaceutically acceptable salt thereof, and a pharmaceutically acceptable carrier.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydronaphthalene is an example of a 10-membered cycloalkyl group.

For compounds of the disclosure in which a variable appears more than once, each variable can be a different moiety independently selected from the group defining the variable. For example, where a structure is described having two R groups that are simultaneously present on the same compound, the two R groups can represent different moieties independently selected from the group defined for R.

The phrase “optionally substituted” means unsubstituted or substituted.

The term “substituted” means that an atom or group of atoms formally replaces hydrogen as a “substituent” attached to another group. The term “substituted” refers, unless otherwise indicated, to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. A single divalent substituent, e.g., oxo, can replace two hydrogen atoms.

The term “Cₙ₋ₘ” where n and m are integers is employed in combination with a chemical group to designate a range of the number of carbon atoms in the chemical group, with n and m defining the range. For example, C₁₋₆ alkyl refers to an alkyl group having 1, 2, 3, 4, 5, or 6 carbon atoms. The term is intended to include each and every member in the indicated range. Thus, Cₙ₋ₘ includes each member in the series Cₙ, Cₙ₊₁, . . . Cₘ₋₁, and Cₘ. Examples include C₁₋₄ (which includes C₁, C₂, C₃, and C₄), C₁₋₆ (which includes C₁, C₂, C₃, C₄, C₅, and C₆) and the like.

The term “alkyl,” employed alone or in combination with other terms, refers to a saturated hydrocarbon group that may be straight-chain or branched. The term “Cₙ₋ₘ alkyl,” refers to an alkyl group having n to m carbon atoms. An alkyl group formally corresponds to an alkane with one C—H bond replaced by the point of attachment of the alkyl group to the remainder of the compound. In some embodiments, the alkyl group contains 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, and the like. In some embodiments, the alkyl group is methyl, ethyl, or propyl.

The term “alkylene,” employed alone or in combination with other terms, refers to a divalent alkyl linking group, which may be straight-chain or branched. An alkylene group formally corresponds to an alkane with two C—H bond replaced by points of attachment of the alkylene group to the remainder of the compound. The term “Cₙ₋ₘ alkylene” refers to an alkylene group having n to m carbon atoms. Examples of alkylene groups include, but are not limited to, methylene, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

The term “alkenyl,” employed alone or in combination with other terms, refers to a straight-chain or branched hydrocarbon group corresponding to an alkyl group having one or more carbon-carbon double bonds. The term “Cₙ₋ₘ alkylenyl” refers to an alkenyl group having n to m carbon atoms. An alkenyl group formally corresponds to an alkene with one C—H bond replaced by the point of attachment of the alkenyl group to the remainder of the compound. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

The term “alkynyl,” employed alone or in combination with other terms, refers to a straight-chain or branched hydrocarbon group corresponding to an alkyl group having one or more carbon-carbon triple bonds. The term “Cₙ₋ₘ alkynyl” refers to an alkynyl group having n to m carbon atoms. An alkynyl group formally corresponds to an alkyne with one C—H bond replaced by the point of attachment of the alkyl group to the remainder of the compound. In some embodiments, the alkynyl moiety contains 2 to 6 or 2 to 4 carbon atoms. Example alkynyl groups include, but are not limited to, ethynyl, propyn-1-yl, propyn-2-yl, and the like.

The term “alkoxy,” employed alone or in combination with other terms, refers to a group of formula —O-alkyl. The term “Cₙ₋ₘ alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms. In some embodiments, the alkyl group has 1 to 6, 1 to 4, or 1 to 3 carbon atoms. Example alkoxy groups include methoxy, ethoxy, propoxy (e.g., n-propoxy and isopropoxy), t-butoxy, and the like. In some embodiments, alkoxy is methoxy.

The term “amino,” employed alone or in combination with other terms, refers to NH₂.

The term “alkylamino,” employed alone or in combination with other terms, refers to a group of formula —NH(alkyl). In some embodiments, the alkylamino group has 1 to 6 or 1 to 4 carbon atoms. Example alkylamino groups include methylamino, ethylamino, propylamino (e.g., n-propylamino and isopropylamino), and the like.

The term “C₁₋₃ alkoxy-C₁₋₃ alkyl” refers to a group of formula —(C₁₋₃ alkylene)-(C₁₋₃ alkoxy).

The term “C₁₋₃ alkoxy-C₁₋₃ alkoxy” refers to a group of formula —(C₁₋₃ alkoxylene)-(C₁₋₃ alkoxy).

The term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms.

The term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms.

The term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

The term “Cₙ₋ₘ alkylcarbonyl” refers to a group of formula —C(O)-alkyl, wherein the alkyl group has n to m carbon atoms. In some embodiments, the alkyl group has 1 to 6, 1 to 4, or 1 to 3 carbon atoms.

The term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

The term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

The term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

The term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

The term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

The term “Cₙ₋ₘ alkylsulfinyl” refers to a group of formula —S(O)-alkyl, wherein the alkyl group has n to m carbon atoms. In some embodiments, the alkyl group has 1 to 6, 1 to 4, or 1 to 3 carbon atoms.

The term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

The term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

The term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

The term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

The term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

The term “aminosulfonyl” refers to a group of formula —S(O)₂NH₂.

The term “aminosulfonylamino” refers to a group of formula —NHS(O)₂NH₂.

The term “aminocarbonylamino”, employed alone or in combination with other terms, refers to a group of formula —NHC(O)NH₂.

The term “carbonyl,” employed alone or in combination with other terms, refers to a —C(═O)— group.

The term “carboxy” refers to a group of formula —C(O)OH.

The term “cyano” or “nitrile” refers to a group of formula —C≡N, which also may be written as —CN.

The term “cyano-C₁₋₃ alkyl” refers to a group of formula —(C₁₋₃ alkylene)-CN.

The term “halo” or “halogen”, employed alone or in combination with other terms, refers to fluoro, chloro, bromo, and iodo. In some embodiments, “halo” refers to a halogen atom selected from F, Cl, or Br. In some embodiments, halo is F or Cl. In some embodiments, halo is F.

The term “haloalkyl,” employed alone or in combination with other terms, refers to an alkyl group in which one or more of the hydrogen atoms has been replaced by a halogen atom, having up to the full valency of halogen atom substituents, which may either be the same or different. In some embodiments, the halogen atoms are fluoro atoms. The term “Cₙ₋ₘ haloalkyl” refers to a Cₙ₋ₘ alkyl group having n to m carbon atoms and from at least one up to {2(n to m)+1}halogen atoms, which may either be the same or different. In some embodiments, the alkyl group has 1 to 6, 1 to 4, or 1 to 3 carbon atoms. Example haloalkyl groups include CF₃, C₂F₅, CHF₂, CCl₃, CHCl₂, C₂Cl₅, and the like. In some embodiments, the haloalkyl group is a fluoroalkyl group.

The term “haloalkoxy,” employed alone or in combination with other terms, refers to a group of formula —O-(haloalkyl). The term “Cₙ₋ₘ haloalkoxy” refers to a haloalkoxy group, the haloalkyl group of which has n to m carbons. In some embodiments, the alkyl group has 1 to 6, 1 to 4, or 1 to 3 carbon atoms. An example haloalkoxy group is —OCF₃.

The term “H₂N—C₁₋₃ alkyl” refers to a group of formula —(C₁₋₃ alkylene)-NH₂.

The term “HO—C₁₋₃ alkoxy” refers to a group of formula —(C₁₋₃ alkoxylene)-OH.

The term “HO—C₁₋₃ alkyl” refers to a group of formula —(C₁₋₃ alkylene)-OH.

The term “oxo” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “oxidized” in reference to a ring-forming N atom refers to a ring-forming N-oxide.

The term “oxidized” in reference to a ring-forming S atom refers to a ring-forming sulfonyl or ring-forming sulfinyl.

The term “thio” refers to a group of formula —SH.

The term “alkylthio,” employed alone or in combination with other terms, refers to a group of formula —S-alkyl. The term “Cₙ₋ₘ alkylthio” refers to a group of formula —S-alkyl, wherein the alkyl group has n to m carbon atoms. In some embodiments, the alkyl group has 1 to 6, 1 to 4 carbon, or 1 to 3 carbon atoms.

The term “aromatic” refers to a carbocycle or heterocycle having one or more polyunsaturated rings having aromatic character (i.e., having (4n+2) delocalized π (pi) electrons where n is an integer).

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. Aryl groups include, e.g., phenyl, naphthyl, and the like. In some embodiments, aryl groups have from 6 to about 10 carbon atoms. In some embodiments, aryl groups have 6 carbon atoms. In some embodiments, aryl groups have 10 carbon atoms. In some embodiments, the aryl group is phenyl. In some embodiments, the aryl group is naphthyl.

The term “heteroaryl” or “heteroaromatic” employed alone or in combination with other terms, refers to a monocyclic or polycyclic (e.g., having 2 or 3 fused rings) aromatic hydrocarbon moiety, having one or more heteroatom ring members independently selected from nitrogen, sulfur and oxygen. In some embodiments, the heteroaryl group is a monocyclic or bicyclic group having 1, 2, 3, or 4 heteroatoms independently selected from nitrogen, sulfur and oxygen. Example heteroaryl groups include, but are not limited to, pyridyl, pyrimidinyl, pyrazinyl, pyridazinyl, triazinyl, furyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, pyrrolyl, azolyl, quinolinyl, isoquinolinyl, benzisoxazolyl, imidazo[1,2-b]thiazolyl, 2H-pyrazolo[4,3-c]pyridinyl, 1H-pyrazolo[3,4-c]pyridinyl, pyridinyl, 3H-imidazo[4,5-c]pyridinyl, 1,6-naphthyridinyl, 2,6-naphthyridinyl, 7H-purinyl, imidazo[1,5-a]pyrazinyl, imidazo[1,5-a]pyrazinyl, pyrazolo[1,5-a]pyrazinyl, imidazo[1,2-c]pyrimidinyl, 1H-pyrazolo[4,3-c]pyridinyl, 1H-imidazolyl, 3H-imidazo[4,5-b]pyridinyl, [1,2,4]triazolo[4,3-a]pyridinyl, pyrido[2,3-d]pyrimidinyl, 1,8-naphthyridinyl, 3a,7a-dihydro-1H-pyrazolo[3,4-d]pyrimidinyl, 1H-imidazo[4,5-c]pyridinyl, or the like. The carbon atoms or heteroatoms in the ring(s) of the heteroaryl group can be oxidized to form a carbonyl, an N-oxide, or a sulfonyl group (or other oxidized linkage) or a nitrogen atom can be quaternized, provided the aromatic nature of the ring is preserved. In some embodiments the heteroaryl group is a 5 to 10 membered heteroaryl group. In another embodiment the heteroaryl group is a 5 to 6 membered heteroaryl group. In some embodiments, the heteroaryl is a 5-6 membered heteroaryl moiety having carbon and 1, 2, or 3 heteroatoms independently selected from N, O and S. In some embodiments, the heteroaryl is a 5-10 membered heteroaryl moiety having carbon and 1, 2, or 3 heteroatoms independently selected from N, O and S. In some embodiments, the heteroaryl has 5-6 ring atoms and 1 or 2 heteroatom ring members independently selected from nitrogen, sulfur and oxygen. In some embodiments, no more than 2 heteroatoms of a 5-membered heteroaryl moiety are N.

A five-membered heteroaryl ring is a heteroaryl group having five ring atoms wherein one or more (e.g., 1, 2 or 3) ring atoms are independently selected from N, O and S. Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S. Exemplary six-membered ring heteroaryls are pyridyl, pyrazinyl, pyrimidinyl, triazinyl, isoindolyl, and pyridazinyl.

A nine-membered heteroaryl ring is a heteroaryl group having nine ring atoms wherein one or more (e.g., 1, 2 or 3) ring atoms are independently selected from N, O and S. Exemplary nine-membered ring heteroaryls include benzofuran, benzo[b]thiophene, 1H-indole, 1H-benzo[d]imidazolyl, benzo[d]oxazolyl, benzo[d]thiazolyl, 1H-pyrrolo[3,2-b]pyridinyl, 1H-imidazo[4,5-b]pyridinyl, 1H-pyrrolo[3,2-c]pyridinyl, 1H-imidazo[4,5-c]pyridinyl, 1H-pyrrolo[2,3-c]pyridinyl, 3H-imidazo[4,5-c]pyridinyl, 1H-pyrrolo[2,3-b]pyridinyl, 3H-imidazo[4,5-b]pyridinyl, imidazo[1,2-a]pyridinyl,imidazo[1,2-a]pyrimidinyl,pyrazolo[1,5-a]pyrimidinyl,[1,2,4]triazolo[1,5-a]pyrimidinyl,[1,2,4]triazolo[1,5-a]pyridinyl,imidazo[1,2-c]pyrimidinyl,pyrrolo[1,2-a]pyrimidinyl, and 2H-pyrazolo[3,4-c]pyridinyl.

A ten-membered heteroaryl ring is a heteroaryl group having ten ring atoms wherein one or more (e.g., 1, 2 or 3) ring atoms are independently selected from N, O and S. Exemplary ten-membered ring heteroaryls are 1,7-naphthyridinyl, 2,7-naphthyridinyl, 3,7-naphthyridinyl, and 4,7-naphthyridinyl.

The term “cycloalkyl” or “cycloalkane” employed alone or in combination with other terms, refers to a non-aromatic cyclic hydrocarbon including cyclized alkyl and alkenyl groups. The terms “Cₙ₋ₘ cycloalkyl” refers to a cycloalkyl that has from n to m ring member carbon atoms. Cycloalkyl groups can include mono- or polycyclic (e.g., having 2, 3, or 4 fused, bridged, or spiro rings) ring systems. Also included are moieties that have one or more aromatic rings (e.g., aryl or heteroaryl rings) fused to (i.e., having a bond in common with) the cycloalkyl ring, for example, benzo derivatives of cyclopentane, cyclohexene, cyclohexane, and the like, or pyrido derivatives of cyclopentane or cyclohexane. A cycloalkyl group containing a fused aromatic ring can be attached through any ring-forming atom including a ring-forming atom of the fused aromatic ring. Ring-forming carbon atoms of a cycloalkyl group can be optionally substituted by oxo. The term “cycloalkyl” also includes bridgehead cycloalkyl groups (e.g., non-aromatic cyclic hydrocarbon moieties containing at least one bridgehead carbon, such as admantan-1-yl) and spirocycloalkyl groups (e.g., non-aromatic hydrocarbon moieties containing at least two rings fused at a single carbon atom, such as spiro[2.5]octane and the like). In some embodiments, the cycloalkyl group has 3 to 10 ring members, or 3 to 7 ring members, or 3 to 6 ring members. In some embodiments, the cycloalkyl group is monocyclic or bicyclic. In some embodiments, the cycloalkyl group is monocyclic. In some embodiments, the cycloalkyl group is a C₃₋₇ monocyclic cycloalkyl group. In some embodiments, the cycloalkyl group is cyclopropyl or cyclohexyl.

The term “heterocycloalkyl,” or “heterocycloalkane” or employed alone or in combination with other terms, refers to a non-aromatic ring or ring system, which has at least one carbon atom ring member and at least one heteroatom ring member independently selected from nitrogen, sulfur, oxygen, and phosphorus, and which has 4-14 ring members, 4-10 ring members, 4-7 ring members, or 4-6 ring members. The ring may contain one or more alkylene, alkenylene or alkynylene groups as part of the ring structure. The term “n-m-membered heterocycloalkyl” where n and m are integers refer to a heterocycloalkyl ring or ring system containing from n to m ring-forming atoms. An n-m-membered heterocycloalkyl include from 1 to m-1 carbon atoms and from 1 to m-1 heteroatoms. The term “n-membered heterocycloalkyl” where n is an integer refers to a heterocycloalkyl ring or ring system containing from n to m ring-forming atoms. Included within the term “heterocycloalkyl” are monocyclic 4-, 5-, 6- and 7-membered heterocycloalkyl. Heterocycloalkyl groups can include mono- or polycyclic (e.g., having 2, 3 or 4 fused, bridged, or spiro rings) or spirocyclic ring systems. In some embodiments, the heterocycloalkyl group is a monocyclic or bicyclic group having 1, 2, 3, or 4 heteroatoms independently selected from nitrogen, sulfur and oxygen. Also included in the definition of heterocycloalkyl are moieties that have one or more aromatic rings (e.g., aryl or heteroaryl rings) fused (i.e., having a bond in common with) to the non-aromatic heterocycloalkyl ring, for example, 1,2,3,4-tetrahydro-quinoline and the like. Heterocycloalkyl groups can also include bridgehead heterocycloalkyl groups (e.g., a heterocycloalkyl moiety containing at least one bridgehead atom, such as azaadmantan-1-yl and the like) and spiroheterocycloalkyl groups (e.g., a heterocycloalkyl moiety containing at least two rings fused at a single atom, such as [1,4-dioxa-8-aza-spiro[4.5]decan-N-yl] and the like). In some embodiments, the heterocycloalkyl group has 3 to 10 ring-forming atoms, 4 to 10 ring-forming atoms, or 3 to 8 ring forming atoms. In some embodiments, the heterocycloalkyl group has 1 to 5 heteroatoms, 1 to 4 heteroatoms, 1 to 3 heteroatoms, or 1 to 2 heteroatoms. The carbon atoms or heteroatoms in the ring(s) of the heterocycloalkyl group can be oxidized to form a carbonyl, an N-oxide, or a sulfonyl group (or other oxidized linkage) or a nitrogen atom can be quaternized. In some embodiments, the heterocycloalkyl portion is a C₂₋₇ monocyclic heterocycloalkyl group. In some embodiments, the heterocycloalkyl group is a morpholine ring, pyrrolidine ring, piperazine ring, piperidine ring, dihydropyran ring, tetrahydropyran ring, tetrahyropyridine, azetidine ring, or tetrahydrofuran ring. In some embodiments, the heterocycloalkyl is a 4-7 membered heterocycloalkyl moiety having carbon and 1, 2, or 3 heteroatoms independently selected from N, O and S. In some embodiments, the heterocycloalkyl is 4-10 membered heterocycloalkyl moiety having carbon and 1, 2, or 3 heteroatoms independently selected from N, O and S.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Resolution of racemic mixtures of compounds can be carried out by methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Other resolving agents suitable for fractional crystallization methods include stereoisomerically pure forms of methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

In some embodiments, the compounds of the disclosure have the (R)-configuration. In other embodiments, the compounds have the (S)-configuration. In compounds with more than one chiral center, each of the chiral centers in the compound may be independently (R) or (S), unless otherwise indicated. In compounds with a single chiral center, the stereochemistry of the chiral center can be (R) or (S). In compounds with two chiral centers, the stereochemistry of the chiral centers can each be independently (R) or (S) so the configuration of the chiral centers can be (R) and (R), (R) and (S); (S) and (R), or (S) and (S). In compounds with three chiral centers, the stereochemistry each of the three chiral centers can each be independently (R) or (S) so the configuration of the chiral centers can be (R), (R) and (R); (R), (R) and (S); (R), (S) and (R); (R), (S) and (S); (S), (R) and (R); (S), (R) and (S); (S), (S) and (R); or (S), (S) and (S).

Compounds of the disclosure also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution. Compounds herein identified by name or structure as one particular tautomeric form are intended to include other tautomeric forms unless otherwise specified (e.g., in the case of purine rings, unless otherwise indicated, if a compound name or structure described the 9H tautomer, it would be understood that the 7H tautomer is also encompassed).

The term, “compound,” is intended to include all stereoisomers, geometric isomers, tautomers, and isotopes of the structures depicted. The term is also meant to refer to compounds of the disclosure, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., in the form of hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the disclosure, or salts thereof, are substantially isolated. By “substantially isolated” is meant that the compound is at least partially or substantially separated from the environment in which it was formed or detected. Partial separation can include, for example, a composition enriched in the compounds of the disclosure. Substantial separation can include compositions containing at least about 50%, at least about 60%, at least about 70%, at least about 80%, at least about 90%, at least about 95%, at least about 97%, or at least about 99% by weight of the compounds of the disclosure, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present disclosure also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, EtOAc, alcohols (e.g., MeOH, EtOH, iso-propanol, or butanol) or MeCN are preferred. Lists of suitable salts are found in A. R. Gennaro (Ed.), Remington's Pharmaceutical Sciences, 17^{th} Ed., (Mack Publishing Company, Easton, 1985), p. 1418, S. M. Berge et al., J. Pharm. Sci., 1977, 66(1), 1-19 and in P. H. Stahl et al., Handbook of Pharmaceutical Salts: Properties, Selection, and Use, 2^{nd} Ed. (Wiley, 2011).

### III. Synthesis

As will be appreciated by those skilled in the art, the compounds provided herein, including salts and stereoisomers thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in P. Kocienski, Protecting Groups, 3^{rd} Ed. (Thieme, 2005); J. Robertson, Protecting Group Chemistry, (Oxford University Press, 2000); M. B. Smith et al., March's Advanced Organic Chemistry: Reactions, Mechanisms, and Structure, 8^{th} Ed. (Wiley, 2020); S. Pétursson, J. Chem. Educ., 1997, 74(11), 1297-303; and P. G. M. Wuts et al., Greene's Protective Groups in Organic Synthesis, 5^{th} Ed., (Wiley, 2014).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography (TLC).

Compounds can be purified by those skilled in the art by a variety of methods, including high performance liquid chromatography (HPLC) (K. F. Blom, et al., J. Combi. Chem. 2004, 6(6), 874-83) and normal phase silica chromatography.

The Scheme below provide general guidance in connection with preparing the compounds of the invention. One skilled in the art would understand that the preparations shown in the Scheme can be modified or optimized using general knowledge of organic chemistry to prepare various compounds of the invention.

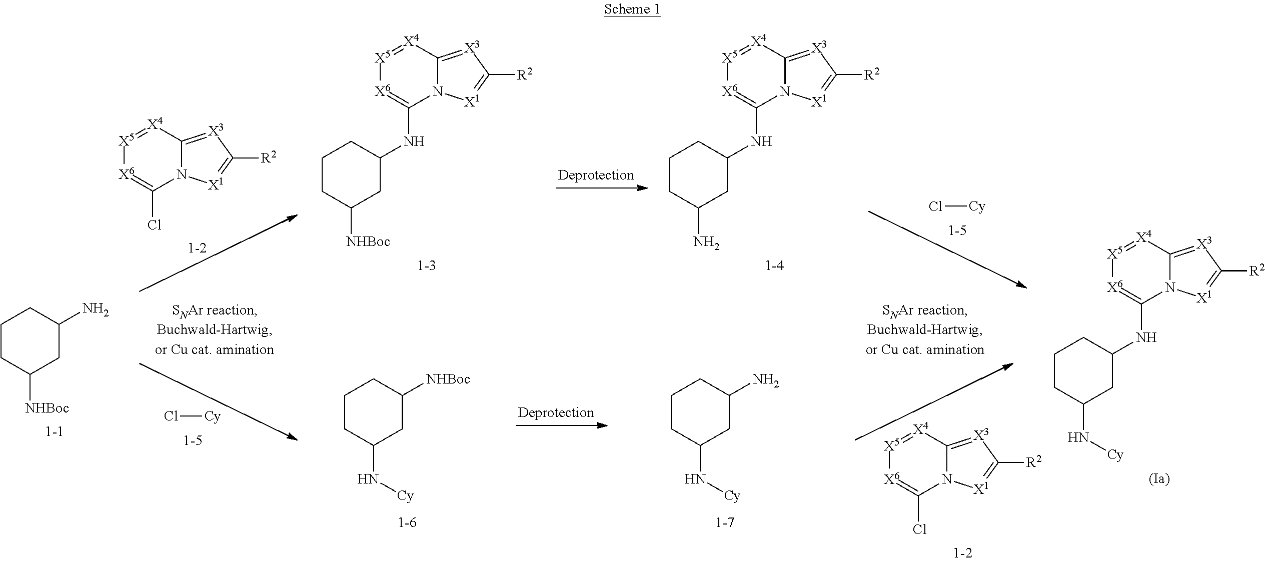

Compounds of Formula Ia such as those described herein can be prepared, using a process as illustrated in Scheme 1. Compound 1-1 can react with compounds of Formula 1-2 via a nucleophilic aromatic substitution (e.g., S_{N}Ar) or a number of different cross-coupling reactions, including Buchwald-Hartwig amination, Cu-catalyzed amination and others, to give the compounds of Formula 1-3. Deprotection of the protecting group, e.g. under acidic conditions, such as treatment with HCl or TFA, results in the formation of the desired compounds of Formula 1-4. Compound 1-4 can react with compounds of Formula 1-5 via a nucleophilic aromatic substitution (e.g., S_{N}Ar) or a number of different cross-coupling reactions, including Buchwald-Hartwig amination, Cu-catalyzed amination and others, to give the compounds of Formula Ia. Compounds of Formula Ia could also be prepared by reaction of compound 1-1 with compounds of Formula 1-5 via a nucleophilic aromatic substitution (e.g., S_{N}Ar) or a number of different cross-coupling reactions, including Buchwald-Hartwig amination, Cu-catalyzed amination and others, yielding the compounds of Formula 1-6. Deprotection of the protecting group, e.g. under acidic conditions, such as treatment with HCl or TFA, results in the formation of the desired compounds of Formula 1-7. Compound 1-7 can react with compounds of Formula 1-2 via a nucleophilic aromatic substitution (e.g., S_{N}Ar) or a number of different cross-coupling reactions, including Buchwald-Hartwig amination, Cu-catalyzed amination and others, to give the compounds of Formula Ia.

For the synthesis of particular compounds, the general schemes described above and specific methods described herein for preparing particular compounds can be modified. For example, the products or intermediates can be modified to introduce particular functional groups. Alternatively, the substituents can be modified at any step of the overall synthesis by methods know to one skilled in the art, e.g., as described by R. C. Larock, et al., Comprehensive Organic Transformations: A Guide to Functional Group Preparations, 3^{rd} Ed. Vols. 1-4 (Wiley, 2018); A. R. Katritzky, et al. (Eds.), Comprehensive Organic Functional Group Transformations, Vols. 1-6 (Pergamon Press, 1995), and A. R. Katritzky et al. (Eds.), Comprehensive Organic Functional Group Transformations II, Vols. 1-6 (Elsevier, 2^{nd} Edition, 2005);

Starting materials, reagents and intermediates whose synthesis is not expressly described herein are either commercially available, known in the literature, or may be prepared by methods known to one skilled in the art.

It will be appreciated by one skilled in the art that the processes described are not the exclusive means by which compounds of the invention may be synthesized and that a broad repertoire of synthetic organic reactions is available to be potentially employed in synthesizing compounds of the invention. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-114 (Elsevier, 1963-2023); Journal of Heterocyclic Chemistry Vols. 1-60 (Journal of Heterocyclic Chemistry, 1964-2023); E. M. Carreira, et al. (Eds.) Science of Synthesis, Vols. 1-48 (2001-2010) and Knowledge Updates KU2010/1-4; 2011/1-4; 2012/1-4, 2013/1-4; 2014/1-4, 2015/1-2; 2016/1-3, 2017/1-3; 2018/1-4, 2019/1-3; 2020/1-3, 2021/1-3, 2022/1-3, 2023/1 (Thieme, 2001-2023); Houben-Weyl, Methoden der Organischen Chemie, 4^{th} Ed. Vols. 1-67 (Thieme, 1952-1987); Houben-Weyl, Methoden der Organischen Chemie, E-Series. Vols. 1-23 (Thieme, 1982-2003); A. R. Katritzky, et al. (Eds.), Comprehensive Organic Functional Group Transformations, Vols. 1-6 (Pergamon Press, 1995); A. R. Katritzky et al. (Eds.), Comprehensive Organic Functional Group Transformations II, Vols. 1-6 (Elsevier, 2^{nd} Edition, 2005); A. R. Katritzky et al. (Eds.); Comprehensive Heterocyclic Chemistry, Vols. 1-8 (Pergamon Press, 1984); A. R. Katritzky, et al. (Eds.); Comprehensive Heterocyclic Chemistry II, Vols. 1-10 (Pergamon Press, 1996); A. R. Katritzky, et al. (Eds.); Comprehensive Heterocyclic Chemistry III, Vols. 1-14 (Elsevier Science, 2008); D. St.C. Black, et al. (Eds.); Comprehensive Heterocyclic Chemistry IV, Vols. 1-14 (Elsevier Science, 2022); M. B. Smith et al., March's Advanced Organic Chemistry: Reactions, Mechanisms, and Structure, 8^{th} Ed. (Wiley, 2020); B. M. Trost et al. (Ed.), Comprehensive Organic Synthesis, Vols. 1-9 (Pergamon Press, 1991); and Patai's Chemistry of Functional Groups, 100 Vols. (Wiley 1964-2022).

### IV. Uses of the Compounds

Compounds of the present disclosure, including the compounds of Formula (I), or any of the embodiments thereof, are useful for therapy as described in further detail below. The present disclosure provides compounds of Formula (I), for use as a medicament, or for use in medicine, as described in further detail below. The present disclosure also provides the use of compounds of Formula (I), or any of the embodiments thereof, as a medicament, or for treating disease, as described in further detail below. The present disclosure also provides the use of compounds of Formula (I), or any of the embodiments thereof, in the manufacture of medicament for treating disease, as described in further detail below.

Compounds of the present disclosure can modulate, antagonize or inhibit the activity of the MRGPRX2 protein. As MRGPRX2 modulators, antagonists or inhibitors, the compounds of the disclosure are useful in the treatment of MRGPRX2 dependent conditions.

The compounds of the present disclosure may be used for treating an MRGPRX2 dependent condition caused by IgE independent activation of MRGPRX2 and that would benefit from modulating MRGPRX2. IgE independent activation of MRGPRX2 is capable of inducing mast cell degranulation and release of inflammatory mediators.

In some embodiments, the MRGPRX2 dependent condition is an itch associated condition, a pain associated condition, a pseudo-allergic reaction, an autoimmune or inflammatory disorder, or cancer-associated condition.

In some embodiments, the MRGPRX2 dependent condition is an itch associated condition, such as chronic itch; senile itch; contact dermatitis; allergic blepharitis; anaphylaxis; anaphylactoid drug reactions; anaphylactic shock; anemia; atopic dermatitis; bullous pemphigoid; candidiasis; chicken pox; end-stage renal failure; hemodialysis; cholestatic pruritus; chronic spontaneous urticaria; chronic inducible urticaria; contact dermatitis, dermatitis herpetiformis; diabetes; drug allergy, dry skin; dyshidrotic dermatitis; ectopic eczema; eosinophilic fasciitis; epidermolysis bullosa; erythrasma; food allergy; folliculitis; fungal skin infection; hemorrhoids; herpes; HIV infection; Hodgkin's disease; hyperthyroidism; iodinated contrast dye allergy; iron deficiency anemia; kidney disease; leukemia, porphyria; lymphoma; mast cell activation syndrome, malignancy; mastocystosis; multiple myeloma; neurodermatitis; onchocerciasis; Paget's disease; pediculosis; polycythemia rubra vera; prurigo nodularis; lichen planus; lichen sclerosis; pruritus ani; pseudo-allergic reactions; pseudorabies; psoriasis; rectal prolapse; sarcoidosis granulomas; scabies; schistosomiasis; scleroderma, severe stress, stasis dermatitis; swimmer's itch; thyroid disease; tinea cruris; uremic pruritus; rosacea; cutaneous amyloidosis; scleroderma; acne; wound healing; burn healing; ocular itch; and urticaria.

In some embodiments, the MRGPRX2 dependent condition is a pain associated condition, such as acute pain, advanced prostate cancer, AIDS-related pain, ankylosing spondylitis, arachnoiditis, arthritis, arthrofibrosis, ataxic cerebral palsy, autoimmune atrophic gastritis, avascular necrosis, back pain, Behcet's disease (syndrome), burning mouth syndrome, bursitis, cancer pain, carpal tunnel, cauda equina syndrome, central pain syndrome, cerebral palsy, cervical stenosis, Charcot-Marie-Tooth (CMT) disease, chronic fatigue syndrome (CFS), chronic functional abdominal pain (CFAP), chronic pain, chronic pancreatitis, chronic pelvic pain syndrome, collapsed lung (pneumothorax), complex regional pain syndrome (CRPS), reflex sympathetic dystrophy syndrome (RDS), corneal neuropathic pain, Crohn's disease, degenerative disc disease, dental pain, Dercum's disease, dermatomyositis, diabetic peripheral neuropathy (DPN), dystonia, Ehlers-Danlos syndrome (EDS), endometriosis, eosinophilia-myalgia syndrome (EMS), erythromelalgia, fibromyalgia, gout, headaches, herniated disc, hydrocephalus, intercostal neuralgia, interstitial cystitis, irritable bowel syndrome (IBS), juvenile dermatositis (dermatomyositis), knee injury, leg pain, loin pain-haematuria syndrome, lupus, Lyme disease, medullary sponge kidney (MSK), meralgia paresthetica, mesothelioma, migraine, musculoskeletal pain, myofascial pain, myositis, neck pain, neuropathic pain, occipital neuralgia, osteoarthritis, Paget's disease, pain crisis in sickle cell disease; Parsonage-Turner syndrome, pelvic pain, periodontitis pain, peripheral neuropathy, phantom limb pain, pinched nerve, polycystic kidney disease, polymyalgia rheumatica, polymyositis, porphyria, post herniorrhaphy pain syndrome, post mastectomy pain, postoperative pain, pain syndrome, post stroke pain, post thoracotomy pain syndrome, postherpetic neuralgia (shingles), post-polio syndrome, primary lateral sclerosis, psoriatic arthritis, pudendal neuralgia, radiculopathy, Raynaud's disease, rheumatoid arthritis (RA), sacroiliac joint dysfunction, sarcoidosis, Scheuermann's kyphosis disease, sciatica, scoliosis, shingles (herpes zoster), Sjögren's syndrome, spasmodic torticollis, sphincter of oddi dysfunction, spinal cerebellum ataxia (SCA ataxia), spinal cord injury, spinal stenosis, syringomyelia, Tarlov cysts, transverse myelitis, trigeminal neuralgia, neuropathic pain, ulcerative colitis, vascular pain and vulvodynia.

In some embodiments, the MRGPRX2 dependent condition is a pseudo-allergic reaction, such as pseudo-allergic reactions caused by secretagogues, cationic peptidergic drugs, anionic peptidergic drugs, neutral peptidergic drugs, non-steroidal anti-inflammatory drugs, neuropeptides, antimicrobial peptides, opioids, neuromuscular blocking agents, antidepressant agents, antipsychotic agents, antihistamine agents, antineoplastic agents, fluoroquinolone and non-fluoroquinolone antibiotics and tyrosine-kinase inhibitors. The phrase “pseudo-allergic reaction” refers to an IgE-independent allergic reaction, characterized by release of histamine and cytokines, activation of the complement system, atypical synthesis of eicosanoids, inflammation, skin flushing, headache, edema, hypotension, urticaria (hives), bronchospasm, or any combination thereof. A pseudo-allergic reaction is a hypersensitivity reaction manifested by systemic responses. The symptoms of pseudo-allergic reaction are identical to anaphylaxis, however their mechanism is non-IgE-mediated. A pseudo-allergic reaction may be caused by a range of cationic substances, collectively called basic secretagogues, including inflammatory peptides and drugs associated with allergic-type reactions. In one embodiment, the pseudo-allergic reaction is caused by MCD peptide, substance P, VIP, PACAP, dynorphin, somatostatin, Compound 48/80, cortistatin-14, mastoparan, melittin, cathelicidin peptides, ciprofloxacin, vancomycin, leuprolide, goserelin, histrelin, triptorelin, cetrorelix, ganirelix, degarelix, octreotide, lanreotide, pasireotide, sermorelin, tesamorelin, icatibant, glatiramer acetate, teriparatide, pramlintide, bleomycin, exenatide, glucagon, liraglutide, enfuvirtide, colistimethate, succinylcholine, tubocurarine, atracurium, mivacurium, and rocuronium.

In some embodiments, the MRGPRX2 dependent condition is an autoimmune disorder or inflammatory condition, such as chronic inflammation, mast cell activation syndrome, multiple sclerosis, Steven Johnson's syndrome, toxic epidermal necrolysis, appendicitis, bursitis, cutaneous lupus, colitis, cystitis, dermatitis, phlebitis, reflex sympathetic dystrophy/complex regional pain syndrome (RSD/CRPS), rhinitis, tendonitis, tonsillitis, acne vulgaris, sinusitis, rosacea, psoriasis, graft-versus-host disease, reactive airway disorder, asthma, airway infection, allergic rhinitis, autoinflammatory disease, celiac disease, chronic prostatitis, diverticulitis, glomerulonephritis, hidradenitis suppurativa, hypersensitivities, intestinal disorder, epithelial intestinal disorder, inflammatory bowel disease, irritable bowel syndrome, Crohn's disease, ulcerative colitis, lupus erythematous, interstitial cystitis, otitis, pelvic inflammatory disease, endometrial pain, reperfusion injury, rheumatic fever, rheumatoid arthritis, sarcoidosis, transplant rejection, psoriasis, lung inflammation, chronic obstructive pulmonary disease, permanent sputum eosinophilia, eosinophilic leukemia, eosinophilic esophagitis, eosinophilic gastritis, eosinophilic duodenitis, eosinophilic gastroenteritis, mast cell gastrointestinal disease, hypereosinophilic syndrome, aspirin-exacerbated respiratory disease, nasal polyposis, chronic rhinosinusitis, antibody-dependent cell-mediated cytotoxicity, neurofibromatosis, schwannomatosis, tubulointerstitial nephritis, glomerulonephritis, diabetic nephropathy, allograft rejection, amyloidosis, renovascular ischemia, reflux nephropathy, polycystic kidney disease, liver fibrosis/cirrhosis, autoimmune liver disease, biliary atresia, acute and chronic hepatitis B and C virus, liver tumors and cancer, alcoholic liver disease, polycystic liver disease, liver cholangiocarcinoma, primary sclerosing cholangitis, primary biliary cholangitis, neuromyelitis optica spectrum disorder, cardiovascular disease, inflammation induced by bacterial or viral infection, inflammation associated with SARS-COV-2 infection or its variants and coronavirus disease 2019 (COVID-19), acute respiratory distress syndrome, pneumonia, long/long-term/chronic COVID, postacute sequelae of COVID-19 (PASC), myalgic encephalomyelitis/chronic fatigue syndrome (ME/CFS “brain fog”) and vasculitis.

In some embodiments, the compounds of the disclosure are useful to treat a cancer/tumor associated condition, such as adenoid cystic carcinoma, adrenal gland tumor, amyloidosis, anal cancer, appendix cancer, astrocytoma, ataxia-telangiectasia, Beckwith-Wiedemann syndrome, cholangiocarcinoma, Birt-Hogg-Dubé syndrome, bone cancer, brain stem glioma, brain tumor, breast cancer (inflammatory, metastatic, male), prostrate, basal cell, melanoma, colon, colorectal, bladder, kidney cancer, lacrimal gland cancer, laryngeal and hypopharyngeal cancer, lung cancer (non-small cell, small cell), leukemia (acute lymphoblastic, acute lymphocytic, acute myeloid, B cell prolymphocytic, chronic lymphocytic, chronic myeloid, chronic T cell lymphocytic, eosinophilic), liver cancer, Li-Fraumeni syndrome, lymphoma (Hodgkin and non-Hodgkin), lynch syndrome, mastocytosis, medulloblastoma, meningioma, mesothelioma, multiple endocrine neoplasia, multiple myeloma, MUTYH-associated polyposis, myelodysplastic syndrome, nasal cavity and paranasal sinus cancer, neuroblastoma, neuroendocrine tumors, neurofibromatosis, penile cancer, parathyroid cancer, ovarian fallopian tube and peritoneal cancer, osteosarcoma, pituitary gland tumor, pleuropulmonary blastoma, oral and oropharyngeal, thyroid, uterine, pancreatic, carney complex, brain and spinal cord cancer, cervical cancer, Cowden syndrome, craniopharyngioma, desmoid tumor, desmoplastic infantile ganglioglioma, ependymoma, esophageal cancer, Ewing sarcoma, eye cancer, eyelid cancer, familial adenomatous polyposis, familial GIST, familial malignant melanoma, familial pancreatic cancer, gallbladder cancer, gastrointestinal stromal tumor, germ cell tumor, gestational trophoblastic disease, head and neck cancer, hereditary breast and ovarian cancer, hereditary diffuse gastric cancer, hereditary, leiomyomatosis and renal cell cancer, hereditary pancreatitis, hereditary papillary renal carcinoma, hereditary mixed polyposis syndrome, HIV/AIDS related cancers, retinoblastoma, rhabdomyosarcoma, salivary gland cancer, Kaposi sarcoma, small bowel cancer, stomach cancer, testicular cancer, thymoma and thymic carcinoma, thyroid cancer, vaginal cancer, culver cancer, Wermer's syndrome and xeroderma pigmentosum.

The MRGPRX2 dependent condition may be selected from the group consisting of abdominal aortic aneurysms, acute contact dermatitis, allergic rhinitis, amyotrophic lateral sclerosis, asthma, atopic dermatitis, autism, cancer, chronic inducible urticaria, chronic itch, chronic obstructive pulmonary disease, chronic spontaneous urticaria, cold urticaria, contact urticaria, coronary artery disease, cough, Crohn's disease, deep vein thrombosis, drug-induced anaphylactic reactions, endometriosis, fibromyalgia, geographic atrophy, idiopathic chronic cough, idiopathic pulmonary fibrosis, inflammatory pain, interstitial cystitis, irritable bowel syndrome, mast cell activation syndrome, mastocytosis, metabolic syndrome, migraine, multiple sclerosis, nasal polyps, neurodermatitis, neuropathic itch, neuropathic pain, obesity, oesophageal reflux, osteoarthritis, periodontitis, prurigo nodularis, pruritus, pseudo-anaphylaxis, psoriasis, rheumatoid arthritis, rosacea, seborrheic dermatitis, sickle cell disease, ulcerative colitis, and ulcers.

The MRGPRX2 dependent condition may be selected from the group consisting of autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, graft-versus-host disease (GvHD), Guillain-Barre syndrome, Hashimoto's encephalitis, Hashimoto's thyroiditis, ankylosing spondylitis, psoriasis, Sjögren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

The MRGPRX2 dependent condition may be selected from the group consisting of acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/Anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticaria, axonal or neuronal neuropathies, Balo disease, Behcet's disease, bullous pemphigoid, cardiomyopathy, Castleman disease, celiac disease, Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibromyalgia, fibrosing alveolitis, giant cell arteritis (temporal arteritis), giant cell myocarditis, glomerulonephritis, Goodpasture's syndrome, granulomatosis with polyangiitis (GPA) (formerly called Wegener's granulomatosis), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, Immunoregulatory lipoproteins, inclusion body myositis, interstitial cystitis, juvenile arthritis, juvenile diabetes (type 1 diabetes), juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (SLE), Lyme disease, chronic, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (pediatric autoimmune neuropsychiatric disorders associated with streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatica, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm & testicular autoimmunity, stiff person syndrome, subacute bacterial endocarditis (SBE), Susac's syndrome, sympathetic ophthalmia, takayasu's arteritis, temporal arteritis/giant cell arteritis, thrombocytopenic purpura (TTP), tolosa-hunt syndrome, transverse myelitis, type 1 diabetes, ulcerative colitis, undifferentiated connective tissue disease (UCTD), uveitis, vasculitis, vesiculobullous dermatosis, vitiligo, or Wegener's granulomatosis (i.e., granulomatosis with polyangiitis (GPA)).

The present disclosure provides a method of treating a disease in a patient. The disease can be a MRGPRX2 dependent condition, including any of the MRGPRX2 dependent conditions described herein. The method comprises administering to the patient in need of the treatment a therapeutically effective amount of a compound of Formula (I), or any of the embodiments thereof. The condition treated can be any of the conditions described herein.

The phrase “MRGPRX2 dependent condition” refers to a condition in which the activation, over sensitization, or desensitization of MRGPRX2 by a natural or synthetic ligand initiates, mediates, sustains, or augments a pathological condition. For example, it is known that some cationic peptidergic drugs cause pseudo-allergic reactions in patients. MRGPRX2 is sensitive to (or activated by) secretagogues, cationic peptidergic drugs, including icatibant, leuprolide, or ganirelix, neutral and anionic peptidergic drugs (e.g., exenatide, glucagon, liraglutide, enfuviritide, colistimethate), neuromuscular blocking agents (atracurium mivacurium), non-steroidal anti-inflammatory drugs, neuropeptides, antimicrobial peptides. Moreover, overexpression of MRGPRX2 and/or overactivity of MRGPRX2 may also render mast cells more susceptible to activation by endogenous and/or exogenous ligands. Without being bound by theory, it is to be understood that by modulating MRGPRX2, pseudo-allergic reactions, itch, pain, inflammatory and autoimmune disorders can be eased.

The term “autoimmune disorder”, or “inflammatory disorder” means a disease or disorder arising from and/or directed against an individual's own tissues or organs, or a co-segregate or manifestation thereof, or resulting condition therefrom. typically, various clinical and laboratory markers of autoimmune diseases may exist including, but not limited to, hypergammaglobulinemia, high levels of autoantibodies, antigen-antibody complex deposits in tissues, clinical benefit from corticosteroid or immunosuppressive treatments, and lymphoid cell aggregates in affected tissues.

The phrase “itch associated condition” means pruritus (including acute and chronic pruritus) associated with any condition. The itch sensation can originate, e.g., from the peripheral nervous system (e.g., dermal or neuropathic itch) or from the central nervous system (e.g., neuropathic, neurogenic or psychogenic itch).

The term “administration” refers to providing a compound, or a pharmaceutical composition comprising the compound as described herein. The compound or composition can be administered by another person to the subject or it can be self-administered by the subject. Non-limiting examples of routes of administration are oral, parenteral (e.g., intravenous), or topical.

The term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

The term “contacting” refers to the bringing together of indicated moieties in an in vitro system or an in vivo system. For example, “contacting” the MRGPRX2 with a compound described herein includes the administration of a compound described herein to an individual or patient, such as a human, having MRGPRX2, as well as, for example, introducing a compound described herein into a sample containing a cellular or purified preparation containing the MRGPRX2.

The term “individual” or “patient,” used interchangeably, refers to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

The phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

The Federal Food, Drug, and Cosmetic Act defines “pediatric” as a subject aged 21 or younger at the time of their diagnosis or treatment. Pediatric subpopulations are further characterized as: (i) neonates—from birth through the first 28 days of life; (ii) infants—from 29 days to less than 2 years; (iii) children—2 years to less than 12 years; and (iv) adolescents—aged 12 through 21. Despite the definition, depending on the susceptible patient population and clinical trial evaluation, an approved regulatory label may include phrasing that specifically modifies the range of a pediatric population, such as, for example, pediatric patients up to 22 years of age.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### V. Combination Therapies

One or more additional pharmaceutical agents or treatment methods can be used in combination with compounds described herein for treatment of MRGPRX2 dependent conditions, as described herein. The agents can be combined with the present compounds in a single dosage form, or the agents can be administered simultaneously or sequentially as separate dosage forms.

In some embodiments, the additional therapeutic agent is an antihistamine, such as an H1 receptor antagonist or an H2 receptor antagonist. In one embodiment, the additional therapeutic agent is an H1 receptor antagonist antihistamine, such as levocetirizine, loratadine, fexofenadine, cetirizine, desloratadine, olopatadine, diphenhydramine, cyproheptadine, hydroxyzine pamoate or ketotifen. In one embodiment, the additional therapeutic agent is a H2 receptor antagonist, such as cimetidine, nizatidine, ranitidine or famotidine. In one embodiment, the additional therapeutic agent is a leukotriene receptor antagonist or leukotriene synthesis inhibitor, such as montelukast, zafirlukast, pranlukast, or 5-lipoxygenase inhibitor (e.g., zileuton, Hypericum perforatum). In one embodiment, the additional therapeutic agent is an immunomodulatory agent such as omalizumab or immunoglobulin therapy. In one embodiment, the additional therapeutic agent is a corticosteroid, such as hydrocortisone, cortisone, betamethasone, triamcinolone, prednisone, prednisolone, or fludrocortisone. In one embodiment, the additional therapeutic agent is a tricyclic antidepressant that can relieve itch such as doxepin, amitriptyline or nortriptyline. In one embodiment, the additional therapeutic agent is an anti-inflammatory drug such as dapsone, sulfasalazine, hydroxychloroquine or colchicine. In one embodiment, the additional therapeutic agent is an immunosuppressant such as cyclosporine, methotrexate, mycophenolic acid or tacrolimus.

### VI. Pharmaceutical Formulations and Dosage Forms

When employed as pharmaceuticals, compounds described herein can be administered in the form of pharmaceutical compositions which refers to a combination of one or more compounds described herein, and at least one pharmaceutically acceptable carrier or excipient. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal, or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, one or more compounds described herein in combination with one or more pharmaceutically acceptable carriers or excipients. In making the compositions described herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is suitable for topical administration.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions described herein can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel KOOLV™). In some embodiments, the polyethylene oxide is polyethylene oxide WSR 1105 (e.g., Polyox WSR 1105™).

In some embodiments, a wet granulation process is used to produce the composition. In some embodiments, a dry granulation process is used to produce the composition.

The compositions can be formulated in a unit dosage form, each dosage containing from, for example, about 5 mg to about 1000 mg, about 5 mg to about 100 mg, about 100 mg to about 500 mg, or about 10 to about 30 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid pre-formulation composition containing a homogeneous mixture of one or more compounds described herein. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above containing from, for example, 0.1 to about 500 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds, or compositions as described herein can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the compound of the disclosure. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of the compounds in a pharmaceutical composition can vary depending upon a number of factors including dosage, chemical characteristics (e.g., hydrophobicity), and the route of administration. For example, compounds of the present disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

Compounds described herein can also be formulated in combination with one or more additional active ingredients, which can include any pharmaceutical agent such as anti-viral agents, vaccines, antibodies, immune enhancers, immune suppressants, anti-inflammatory agents and the like.

### VII. Labelled Compounds

Compounds of the disclosure also include all isotopes of atoms occurring in the intermediates or final compounds. Isotopes include those atoms having the same atomic number but different mass numbers.

The present disclosure further includes isotopically-labelled compounds of the disclosure. An “isotopically-labelled” is a compound of the disclosure where one or more atoms are replaced or substituted by an atom having an atomic mass or mass number different from the atomic mass or mass number typically found in nature (i.e., naturally occurring). A “radio-labelled” compound is an isotopically-labelled compound in which one or more atoms are replaced or substituted by an atom of an isotope that is radioactive.

Suitable isotopes that may be incorporated in compounds of the present disclosure include but are not limited to ²H (also written as D for deuterium), ³H (also written as T for tritium), ¹¹C, ¹³C, ¹⁴C, ¹⁴N, ¹³N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups in Formula (I) can be perdeuterated. The symbol D included in a chemical formula or as a substituent indicates that deuterium is incorporated in the position labelled at greater than natural abundance, and typically indicates an abundance of equal to or greater than 50%, preferably equal to or greater than 90% or equal to or greater than 95%, 96%, 97%, 98%, 99%, 99.5%, 99.9%, 99.95%, or 99.99% relative to other forms of hydrogen.

One or more constituent atoms of the compounds presented herein can be replaced or substituted with isotopes of the atoms in natural or non-natural abundance. In some embodiments, the compound includes at least one deuterium atom. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms.

Synthetic methods for including isotopes into organic compounds are known in the art (A. F. Thomas, Deuterium Labeling in Organic Chemistry, (Appleton-Century-Crofts, New York, N.Y., 1971); J. Atzrodt, et al., Angew. Chem. Int. Ed., 2007, 7744-65; J. R. Hanson, The Organic Chemistry of Isotopic Labelling, (Royal Society of Chemistry, 2011)). Isotopically labelled compounds can used in various studies such as NMR spectroscopy, metabolism experiments, and/or assays.

Substitution with heavier isotopes such as deuterium, may afford certain therapeutic advantages resulting from greater metabolic stability, for example, increased in vivo half-life or reduced dosage requirements, and hence may be preferred in some circumstances. (A. Kerekes, et al., J. Med. Chem. 2011, 54(1), 201-10; R. Xu et al., J. Label. Compd. Radiopharm. 2015, 58, 308-12).

The radionuclide that is incorporated in the instant radio-labelled compounds will depend on the specific application of that radio-labelled compound. For example, for in vitro adenosine receptor labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labelled” or “labelled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

### VIII. Kits

The present disclosure also includes pharmaceutical kits useful, for example, in the treatment of MRGPRX2 dependent conditions, as described herein, which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

Experimental procedures for compounds of the disclosure are provided below. Preparatory LCMS purifications of some of the compounds prepared were performed on Waters mass-directed fractionation systems. The basic equipment setup, protocols, and control software for the operation of these systems have been described in detail in the literature. See e.g., K. F. Blom, J. Combi. Chem., 2002, 4(4), 295-301; K. F. Blom, et al., J. Combi. Chem., 2003, 5(5), 670-683; and K. F. Blom, et al., J. Combi. Chem. 2004, 6(6), 874-83. The compounds separated were typically subjected to analytical liquid chromatography mass spectrometry (LCMS) for purity analysis under the following conditions: Instrument; Agilent 1100 series, LC/MSD, Column: Waters SUNFIRE® C₁₈ 5 μm, 2.1×50 mm, Buffers: mobile phase A: 0.025% aq. TFA and mobile phase B: MeCN; gradient 2% to 80% of B in 3 min. with flow rate 2.0 mL/min.

Some of the compounds prepared were also separated on a preparative scale by reverse-phase high performance liquid chromatography (RP-HPLC) with MS detector or FCC (silica gel) as indicated in the Examples. Typical preparative reverse-phase high performance liquid chromatography (RP-HPLC) column conditions are as follows:

pH=2 purifications: Waters SUNFIRE® C₁₈ 5 μm, 19×100 mm column, eluting with mobile phase A: 0.1% aq. TFA and mobile phase B: MeCN; the flow rate was 30 mL/min., the separating gradient was optimized for each compound using the Compound Specific Method Optimization protocol as described in the literature [see K. F. Blom, et al., J. Combi. Chem. 2004, 6(6), 874-83]. Typically, the flow rate used with the 30×100 mm column was 60 mL/min.

pH=10 purifications: Waters XBRIDGE® C₁₈ 5 μm, 19×100 mm column, eluting with mobile phase A: 0.15% aq. NH₄OH and mobile phase B: MeCN; the flow rate was 30 mL/min., the separating gradient was optimized for each compound using the Compound Specific Method Optimization protocol as described in the literature [See K. F. Blom, et al., J. Combi. Chem. 2004, 6(6), 874-83]. Typically, the flow rate used with 30×100 mm column was 60 mL/min.

### Example 1. (1R,3S)—N¹-(3-Methyl-3H-imidazo[4,5-c]pyridin-4-yl)-N³-(2-(trifluoromethyl)imidazo[1,2-a]pyridin-5-yl)cyclohexane-1,3-diamine

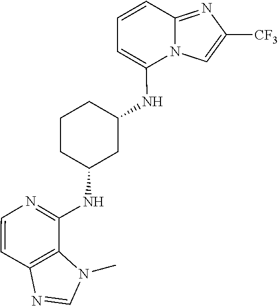

### Step 1. (1R,3S)—N₁-(3-Methyl-3H-imidazo[4,5-c]pyridin-4-yl)cyclohexane-1,3-diamine

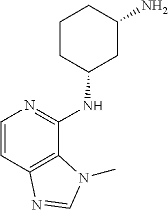 [SMILES: Cn1cnc2ccnc(N[C@@H]3CCC[C@H](N)C3)c21]

A mixture of tert-butyl ((1S,3R)-3-aminocyclohexyl)carbamate (2.88 g, 13.43 mmol), 4-chloro-3-methyl-3H-imidazo[4,5-c]pyridine (1.50 g, 8.95 mmol), tBuBrettPhos Pd G3 (1.148 g, 1.343 mmol), and NaOtBu (1.720 g, 17.90 mmol) in 1,4-dioxane (44.8 mL) was heated at 100° C. for 3 h. The mixture was then allowed to cool to r.t., diluted with EtOAc and filtered through diatomaceous earth. The filtrate was washed with water and the organic layer was concentrated. The concentrate was purified by FCC (MeOH/DCM) to obtain tert-butyl ((1S,3R)-3-((3-methyl-3H-imidazo[4,5-c]pyridin-4-yl)amino)cyclohexyl)carbamate. The product was then taken up in DCM (20 mL) and TFA (10 mL) was added. The mixture was stirred at r.t. for 2 h. The mixture was then concentrated and purified by FCC (MeOH/DCM) to obtain the sub-title compound (1R,3S)—N¹-(3-methyl-3H-imidazo[4,5-c]pyridin-4-yl)cyclohexane-1,3-diamine (442 mg, 1.80 mmol, 20.1% yield). LCMS calc. for C₁₃H₂₀BrN₅ (M+H)⁺: m/z=246.2; found: 246.2.

### Step 2. (1R,3S)—N¹-(3-Methyl-3H-imidazo[4,5-c]pyridin-4-yl)-N³-(2-(trifluoromethyl)imidazo[1,2-a]pyridin-5-yl)cyclohexane-1,3-diamine

A mixture of (1R,3S)—N¹-(3-methyl-3H-imidazo[4,5-c]pyridin-4-yl)cyclohexane-1,3-diamine (15 mg, 0.061 mmol), 5-chloro-2-(trifluoromethyl)imidazo[1,2-a]pyridine (20.2 mg, 0.092 mmol), BrettPhos Pd G3 (8.3 mg, 9.17 μmol), and NaOtBu (11.8 mg, 0.122 mmol) in 1,4-dioxane (1 mL) was heated at 100° C. After 3 h, the mixture was allowed to cool to r.t. and purified by prep.-LCMS (XBRIDGE® C₁₈ column, eluting with a gradient of MeCN/water containing 0.1% TFA, at flow rate of 60 mL/min) to obtain the title compound. LCMS calc. for C₂₁H₂₃F₃N₇ (M+H)⁺: m/z=430.2; found: 430.2.

### Example 2. (1s,4s)-N¹-(3-Methyl-3H-imidazo[4,5-c]pyridin-4-yl)-N⁴-(2-(trifluoromethyl)imidazo[1,2-a]pyridin-5-yl)cyclohexane-1,4-diamine

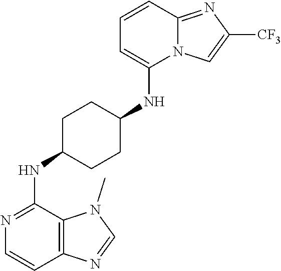 [SMILES: Cn1cnc2ccnc(N[C@H]3CC[C@@H](Nc4cccc5nc(C(F)(F)F)cn45)CC3)c21]

The title compound was prepared according to the procedure described in Example 1, using tert-butyl ((1s,4s)-4-aminocyclohexyl)carbamate instead of tert-butyl ((1S,3R)-3-aminocyclohexyl)carbamate as a starting material. LC-MS calc. for C₂₁H₂₃F₃N₇ (M+H)⁺: m/z=430.2; found 430.3.

### Example 3. (1R,3S)—N¹-(1,6-Naphthyridin-5-yl)-N³-(2-(trifluoromethyl)imidazo[1,2-a]pyridin-5-yl)cyclohexane-1,3-diamine

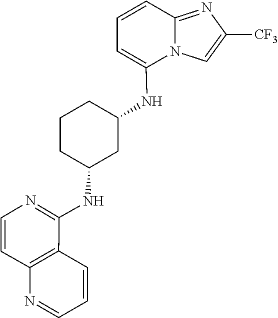

### Step 1. (1R,3S)—N¹-(1,6-Naphthyridin-5-yl)cyclohexane-1,3-diamine

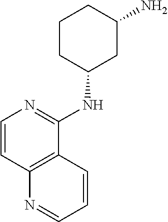

A mixture of tert-butyl ((1S,3R)-3-aminocyclohexyl)carbamate (1455 mg, 6.79 mmol), 5-chloro-1,6-naphthyridine (931 mg, 5.66 mmol), and DIPEA (1.98 mL, 11.3 mmol) in DMSO (20 mL) was heated at 120° C. After 3 days, the mixture was allowed to cool to r.t. and water was added. The precipitated solid was filtered and air-dried to obtain tert-butyl ((1S,3R)-3-((1,6-naphthyridin-5-yl)amino)cyclohexyl)carbamate. The product was then taken up in DCM (30 mL) and TFA (15 mL) was added. The mixture was stirred at r.t. for 20 h. The mixture was then concentrated and purified by FCC (MeOH/DCM) to obtain (1R,3S)—N¹-(1,6-naphthyridin-5-yl)cyclohexane-1,3-diamine (344 mg, 1.42 mmol, 25.1% yield). LCMS calc. for C₁₄H₁₉N₄ (M+H)⁺: m/z=243.2; found: 243.2.

### Step 2. (1R,3S)—N¹-(1,6-Naphthyridin-5-yl)-N³-(2-(trifluoromethyl)imidazo[1,2-a]pyridin-5-yl)cyclohexane-1,3-diamine

The title compound was prepared according to the procedure described in Example 1, Step 2, using (1R,3S)—N¹-(1,6-naphthyridin-5-yl)cyclohexane-1,3-diamine instead of (1R,3S)—N¹-(3-methyl-3H-imidazo[4,5-c]pyridin-4-yl)cyclohexane-1,3-diamine as a starting material. LC-MS calc. for C₂₂H₂₂F₃N₆ (M+H)⁺: m/z=427.2; found 427.2. ¹H NMR (500 MHz, DMSO-d₆) δ 8.90 (dd, J=4.3, 1.6 Hz, 1H), 8.76-8.71 (m, 1H), 8.65 (s, 1H), 8.08 (d, J=5.9 Hz, 1H), 7.49 (dd, J=8.4, 4.3 Hz, 1H), 7.41 (d, J=7.6 Hz, 1H), 7.36 (dd, J=8.9, 7.7 Hz, 1H), 6.97-6.89 (m, 2H), 6.66 (d, J=7.6 Hz, 1H), 6.20 (d, J=7.7 Hz, 1H), 4.38-4.28 (m, 1H), 3.73-3.65 (m, 1H), 2.48-2.42 (m, 1H), 2.15-2.03 (m, 2H), 1.93-1.84 (m, 1H), 1.63-1.49 (m, 2H), 1.43-1.33 (m, 2H) ppm.

### Example 4. (1R,3S)—N¹-(Imidazo[1,5-a]pyrazin-8-yl)-N³-(2-(trifluoromethyl)imidazo[1,2-a]pyridin-5-yl)cyclohexane-1,3-diamine

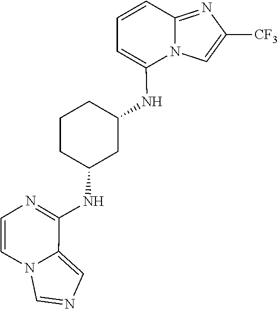

The title compound was prepared according to the procedure described in Example 3, using 8-chloroimidazo[1,5-a]pyrazine instead of 5-chloro-1,6-naphthyridine as a starting material. LC-MS calc. for C₂₀H₂₁F₃N₇ (M+H)⁺: m/z=416.2; found 416.2.

### Example 5. (1s,4s)-N¹-(Imidazo[1,5-a]pyrazin-8-yl)-N⁴-(2-(trifluoromethyl)imidazo[1,2-a]pyridin-5-yl)cyclohexane-1,4-diamine

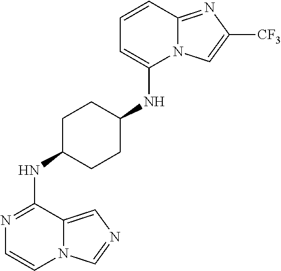 [SMILES: FC(F)(F)c1cn2c(N[C@H]3CC[C@@H](Nc4nccn5cncc45)CC3)cccc2n1]

The title compound was prepared according to the procedure described in Example 4, using tert-butyl ((1s,4s)-4-aminocyclohexyl)carbamate instead of tert-butyl ((1S,3R)-3-aminocyclohexyl)carbamate as a starting material. LC-MS calc. for C₂₀H₂₁F₃N₇ (M+H)⁺: m/z=416.2; found 416.2.

### Example 6. (1R,3S)—N¹-(3-Methyl-3H-imidazo[4,5-c]pyridin-4-yl)-N³-(2-methyl-5-(trifluoromethyl)pyrazolo[1,5-a]pyrimidin-7-yl)cyclohexane-1,3-diamine

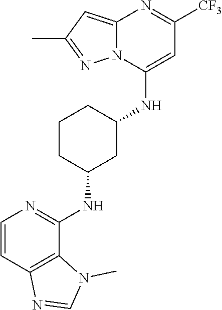 [SMILES: Cc1cc2nc(C(F)(F)F)cc(N[C@H]3CCC[C@@H](Nc4nccc5ncn(C)c45)C3)n2n1]

### Step 1. 5,7-Diiodo-2-methylpyrazolo[1,5-a]pyrimidine

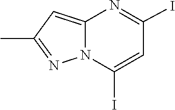

A suspension of 5,7-dichloro-2-methylpyrazolo[1,5-a]pyrimidine (975 mg, 4.83 mmol) and NaI (7234 mg, 48.3 mmol) in MeCN (10 mL) was added acetyl chloride (0.515 mL, 7.24 mmol), and the mixture was stirred at 80° C. After 3 h, the mixture was allowed to cool to r.t., then quenched by the addition of sat. aq. K₂CO₃, aq. Na₂SO₃, and aq. Na₂S₂O₃. The mixture was then diluted with DCM and the organic layer was separated, washed with brine, dried with Na₂SO₄, filtered and concentrated. The obtained solid was used directly in the next step. LCMS calc. for C₇H₆I₂N₃(M+H)⁺: m/z=385.9; found: 385.9.

### Step 2. tert-Butyl ((1R,3S)-3-((5-iodo-2-methylpyrazolo[1,5-a]pyrimidin-7-yl)amino)cyclohexyl)carbamate

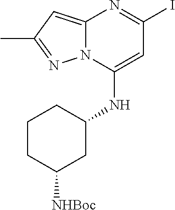 [SMILES: Cc1cc2nc(I)cc(N[C@H]3CCC[C@@H](NC(=O)OC(C)(C)C)C3)n2n1]

A mixture of 5,7-diiodo-2-methylpyrazolo[1,5-a]pyrimidine (898 mg, 2.333 mmol), tert-butyl ((1R,3S)-3-aminocyclohexyl)carbamate (500.0 mg, 2.333 mmol), and DIPEA (0.815 mL, 4.67 mmol) in DMSO (10 mL) was heated at 100° C. After 2 h, the mixture was allowed to cool to r.t., diluted with DCM and water. The organic layer was separated and the crude product was purified by FCC (EtOAc/Hex) to obtain the sub-title compound. LCMS calc. for C₁₈H₂₇IN₅O₂ (M+H)⁺: m/z=472.1; found: 472.2.

### Step 3. (1S,3R)—N¹-(2-Methyl-5-(trifluoromethyl)pyrazolo[1,5-a]pyrimidin-7-yl)cyclohexane-1,3-diamine

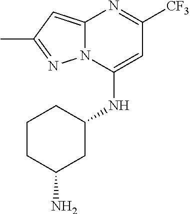 [SMILES: Cc1cc2nc(C(F)(F)F)cc(N[C@H]3CCC[C@@H](N)C3)n2n1]

A mixture of tert-butyl ((1R,3S)-3-((5-iodo-2-methylpyrazolo[1,5-a]pyrimidin-7-yl)amino)cyclohexyl)carbamate (716 mg, 1.519 mmol), methyl 2,2-difluoro-2-(fluorosulfonyl)acetate (0.580 mL, 4.56 mmol), copper(II) chloride (30.6 mg, 0.228 mmol) in DMF (5 mL) was heated at 110° C. for 2 h. The reaction was then allowed to cool to r.t., diluted with EtOAc and water. The organic layer was separated and the crude product was purified by FCC (EtOAc/Hex) to obtain tert-butyl ((1R,3S)-3-((2-methyl-5-(trifluoromethyl)pyrazolo[1,5-a]pyrimidin-7-yl)amino)cyclohexyl)carbamate. The product was then taken up in DCM (5 mL) and TFA (3 mL), and the mixture was stirred at r.t. for 1 h. Solvent was removed and the crude product was then purified by FCC (MeOH/DCM) to obtain (1S,3R)—N¹-(2-methyl-5-(trifluoromethyl)pyrazolo[1,5-a]pyrimidin-7-yl)cyclohexane-1,3-diamine. LCMS calc. for C₁₄H₁₉F₃N₅ (M+H)⁺: m/z=314.2; found: 314.1.

### Step 4. (1R,3S)—N¹-(3-Methyl-3H-imidazo[4,5-c]pyridin-4-yl)-N³-(2-methyl-5-(trifluoromethyl)pyrazolo[1,5-a]pyrimidin-7-yl)cyclohexane-1,3-diamine

A mixture of (1S,3R)—N¹-(2-methyl-5-(trifluoromethyl)pyrazolo[1,5-a]pyrimidin-7-yl)cyclohexane-1,3-diamine (13 mg, 0.041 mmol), 4-chloro-3-methyl-3H-imidazo[4,5-c]pyridine (10.4 mg, 0.062 mmol), NaOtBu (8.0 mg, 0.083 mmol), and tBuBrettPhos Pd G3 (5.3 mg, 6.22 μmol) in 1,4-dioxane (1 mL) was heated at 100° C. After 3 h, the mixture was allowed to cool to r.t. and purified by prep.-LCMS (XBRIDGE® C₁₈ column, eluting with a gradient of MeCN/water containing 0.1% TFA, at flow rate of 60 mL/min) to obtain the title compound. LCMS calc. for C₂₁H₂₄F₃N₈ (M+H)⁺: m/z=445.2; found: 445.3.

### Example 7. (1S,3R)—N¹-(2-Methyl-5-(trifluoromethyl)pyrazolo[1,5-a]pyrimidin-7-yl)-N³-(1,6-naphthyridin-5-yl)cyclohexane-1,3-diamine

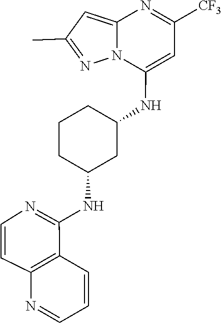

The title compound was prepared according to the procedure described in Example 6, using 5-chloro-1,6-naphthyridine instead of 4-chloro-3-methyl-3H-imidazo[4,5-c]pyridine as a starting material. LC-MS calc. for C₂₂H₂₃F₃N₇ (M+H)⁺: m/z=442.2; found 442.2.

### Example 8. (1S,3R)—N¹-(2-Bromo-5-(trifluoromethyl)pyrazolo[1,5-a]pyrimidin-7-yl)-N³-(pyrazolo[1,5-a]pyrazin-4-yl)cyclohexane-1,3-diamine

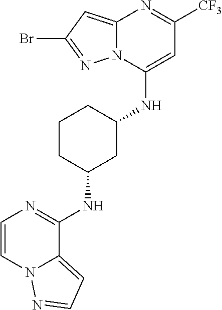 [SMILES: FC(F)(F)c1cc(N[C@H]2CCC[C@@H](Nc3nccn4nccc34)C2)n2nc(Br)cc2n1]

### Step 1. 2-Bromo-5,7-dichloropyrazolo[1,5-a]pyrimidine

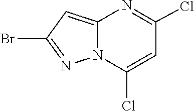 [SMILES: Clc1cc(Cl)n2nc(Br)cc2n1]

A mixture of 3-bromo-1H-pyrazol-5-amine (3.00 g, 18.52 mmol) and sodium ethoxide (3.78 g, 55.6 mmol) in diethyl malonate (30.0 mL, 197 mmol) was heated at 120° C. After 20 h, the mixture was allowed to cool to r.t. and the precipitated solid was filtered, washed with Et₂O and air-dried. The obtained solid was then taken up in phenylphosphonic dichloride (25.0 mL, 178 mmol) and heated at 120° C. After 20 h, the mixture was allowed to cool to r.t. and slowly quenched with sat. aq. NaHCO₃ in an ice bath until pH=7. The mixture was then diluted with DCM and the organic layer was separated, washed with brine and concentrated. The crude product was taken up in Et₂O and the precipitated solid was filtered. The filtrate containing desired product was concentrated and purified by FCC (EtOAc/Hex) to obtain 2-bromo-5,7-dichloropyrazolo[1,5-a]pyrimidine (2.39 g, 8.95 mmol, 48.4% yield) as a yellow solid. LCMS calc. for C₆H₃BrCl₂N₃(M+H)⁺: m/z=265.9; found: 265.9.

### Step 2. 2-Bromo-5,7-diiodopyrazolo[1,5-a]pyrimidine

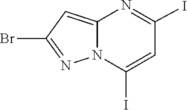 [SMILES: Brc1cc2nc(I)cc(I)n2n1]

A suspension of 2-bromo-5,7-dichloropyrazolo[1,5-a]pyrimidine (1.00 g, 3.75 mmol) and NaI (5.62 g, 37.5 mmol) in MeCN (25 mL) was added acetyl chloride (0.346 mL, 4.87 mmol), and the mixture was stirred at 80° C. After 3 h, the mixture was allowed to cool to r.t., and quenched by addition of sat. aq. K₂CO₃, aq. Na₂SO₃, and aq. Na₂S₂O₃. The mixture was then diluted with DCM and the organic layer was separated, washed with brine, dried with Na₂SO₄, filtered and concentrated. The obtained solid was used directly in the next step. LCMS calc. for C₆H₃BrI₂N₃(M+H)⁺: m/z=449.8; found: 449.7.

### Step 3. tert-Butyl ((1R,3S)-3-((2-bromo-5-iodopyrazolo[1,5-a]pyrimidin-7-yl)amino)cyclohexyl)carbamate

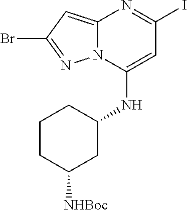 [SMILES: CC(C)(C)OC(=O)N[C@@H]1CCC[C@H](Nc2cc(I)nc3cc(Br)nn23)C1]

A mixture of 2-bromo-5,7-diiodopyrazolo[1,5-a]pyrimidine (1.685 g, 3.75 mmol), tert-butyl ((1R,3S)-3-aminocyclohexyl)carbamate (700.0 mg, 3.27 mmol), and DIPEA (1.14 mL, 6.53 mmol) in DMSO (15 mL) was heated at 100° C. for 2 h. The mixture was then allowed to cool to r.t. Water was added and the precipitated solid was filtered, washed with water and dried to obtain the sub-title compound, which was used directly in the next step. LCMS calc. for C₁₇H₂₄BrIN₅O₂(M+H)⁺: m/z=536.0; found: 536.0.

### Step 4. (1S,3R)—N¹-(2-Bromo-5-(trifluoromethyl)pyrazolo[1,5-a]pyrimidin-7-yl)cyclohexane-1,3-diamine

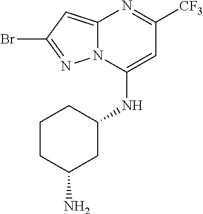 [SMILES: N[C@@H]1CCC[C@H](Nc2cc(C(F)(F)F)nc3cc(Br)nn23)C1]

A mixture of tert-butyl ((1R,3S)-3-((2-bromo-5-iodopyrazolo[1,5-a]pyrimidin-7-yl)amino)cyclohexyl)carbamate (1.75 g, 3.26 mmol), methyl 2,2-difluoro-2-(fluorosulfonyl)acetate (1.25 mL, 9.79 mmol), and copper(II) chloride (66.0 mg, 0.49 mmol) in DMF (16 mL) was heated at 110° C. for 3 h. The mixture was then allowed to cool to r.t., diluted with EtOAc and water. The organic layer was separated and the crude product was purified by FCC (EtOAc/Hex) to obtain tert-butyl ((1R,3S)-3-((2-bromo-5-(trifluoromethyl)pyrazolo[1,5-a]pyrimidin-7-yl)amino)cyclohexyl)carbamate. The product was then taken up in DCM (10 mL) and TFA (5 mL) was added, and the mixture was stirred at r.t. for 1 h. Solvent was removed the crude product was then purified by FCC (MeOH/DCM) to obtain (1S,3R)—N¹-(2-bromo-5-(trifluoromethyl)pyrazolo[1,5-a]pyrimidin-7-yl)cyclohexane-1,3-diamine (1.23 g, 3.26 mmol, 68.9% yield). LCMS calc. for C₁₃H₁₆BrF₃N₅(M+H)⁺: m/z=378.1; found: 378.1.

### Step 5. (1S,3R)—N¹-(2-Bromo-5-(trifluoromethyl)pyrazolo[1,5-a]pyrimidin-7-yl)-N³-(pyrazolo[1,5-a]pyrazin-4-yl)cyclohexane-1,3-diamine

A mixture of (1S,3R)—N¹-(2-bromo-5-(trifluoromethyl)pyrazolo[1,5-a]pyrimidin-7-yl)cyclohexane-1,3-diamine (20.0 mg, 0.053 mmol), 4-chloropyrazolo[1,5-a]pyrazine (12.2 mg, 0.079 mmol), and DIPEA (0.028 mL, 0.16 mmol) in DMSO (1 mL) was heated at 100° C. for 2 days. The mixture was then allowed to cool to r.t. and purified by prep.-LCMS (XBRIDGE® C₁₈ column, eluting with a gradient of MeCN/water containing 0.1% TFA, at flow rate of 60 mL/min) to obtain the title compound. LCMS calc. for C₁₉H₁₉BrF₃N₈(M+H)⁺: m/z=495.1; found: 495.1.

### Example 9. (1S,3R)—N¹-(2-Bromo-5-(trifluoromethyl)pyrazolo[1,5-a]pyrimidin-7-yl)-N³-(1,6-naphthyridin-5-yl)cyclohexane-1,3-diamine

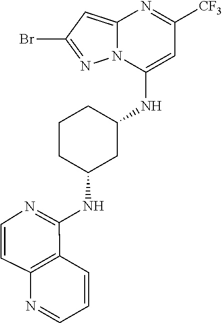

The title compound was prepared according to the procedure described in Example 8, using 5-chloro-1,6-naphthyridine instead of 4-chloropyrazolo[1,5-a]pyrazine as a starting material. LC-MS calc. for C₂₁H₂₀BrF₃N₇(M+H)⁺: m/z=506.1; found 506.1.

### Example 10. (1R,3S)—N¹—([1,2,4]Triazolo[4,3-a]pyridin-3-yl)-N³-(2-bromo-5-(trifluoromethyl)pyrazolo[1,5-a]pyrimidin-7-yl)cyclohexane-1,3-diamine

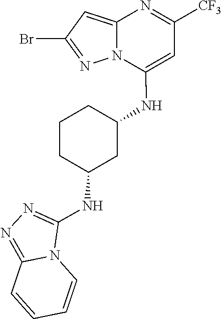 [SMILES: FC(F)(F)c1cc(N[C@H]2CCC[C@@H](Nc3nnc4ccccn34)C2)n2nc(Br)cc2n1]

A mixture of(1S,3R)—N¹-(2-bromo-5-(trifluoromethyl)pyrazolo[1,5-a]pyrimidin-7-yl)cyclohexane-1,3-diamine (20.0 mg, 0.053 mmol), 3-chloro-[1,2,4]triazolo[4,3-a]pyridine (16.2 mg, 0.106 mmol), NaOtBu (10.2 mg, 0.106 mmol), and tBuBrettPhos Pd G3 (6.8 mg, 7.9 μmol) in 1,4-dioxane (1 mL) was heated at 100° C. for 3 h. The mixture was then allowed to cool to r.t. and purified by prep.-LCMS (XBRIDGE® C₁₈ column, eluting with a gradient of MeCN/water containing 0.1% TFA, at flow rate of 60 mL/min) to obtain the title compound. LC-MS calc. for C₁₉H₁₉BrF₃N₈(M+H)⁺: m/z=495.1; found 495.1. ¹H NMR (500 MHz, DMSO-d₆) δ 8.70 (dd, J=9.2, 5.6 Hz, 1H), 8.50 (d, J=7.1 Hz, 1H), 7.82-7.62 (m, 3H), 7.19 (t, J=6.8 Hz, 1H), 6.85 (s, 1H), 6.83 (s, 1H), 4.12-4.00 (m, 1H), 3.93-3.81 (m, 1H), 2.35-2.29 (m, 1H), 2.18-2.12 (m, 1H), 1.95-1.21 (m, 6H) ppm.

### Example 11. (1S,3R)—N¹-(2,5-Bis(trifluoromethyl)pyrazolo[1,5-a]pyrimidin-7-yl)-N³-(1,6-naphthyridin-5-yl)cyclohexane-1,3-diamine

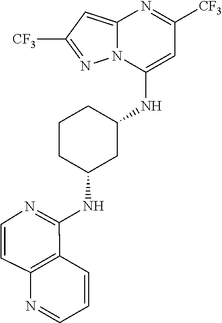

### Step 1. 2,5-Bis(trifluoromethyl)pyrazolo[1,5-a]pyrimidin-7-ol

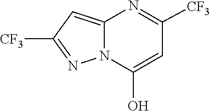 [SMILES: Oc1cc(C(F)(F)F)nc2cc(C(F)(F)F)nn12]

A mixture of 3-(trifluoromethyl)-1H-pyrazol-5-amine (30.0 g, 199.0 mmol), ethyl 4,4,4-trifluoro-3-oxobutanoate (47.5 g, 258.0 mmol), and TFA (3.06 mL, 39.7 mmol) in DMSO (300 mL, 197 mmol) was stirred at r.t. for 2 days. Water was then added and the precipitated solid was filtered, washed with water and air-dried to obtain the sub-title compound. LCMS calc. for C₈H₄F₆N₃O (M+H)⁺: m/z=272.0; found: 272.0.

### Step 2. (1S,3R)—N¹-(2,5-Bis(trifluoromethyl)pyrazolo[1,5-a]pyrimidin-7-yl)-N³-(1,6-naphthyridin-5-yl)cyclohexane-1,3-diamine

To a solution of 2,5-bis(trifluoromethyl)pyrazolo[1,5-a]pyrimidin-7-ol (20.0 mg, 0.074 mmol) and DIPEA (0.052 mL, 0.30 mmol) in DCM (1 mL) was added trifluoromethanesulfonic anhydride solution (1.0 M in DCM, 0.110 mL, 0.110 mmol), and the resulting solution was stirred at r.t. for 10 min. A solution of (1R,3S)—N¹-(1,6-naphthyridin-5-yl)cyclohexane-1,3-diamine (26.8 mg, 0.111 mmol) in DCM (1 mL) was then added and the resulting solution was stirred at r.t. for 30 min. The mixture was then purified by prep.-LCMS (XBRIDGE® C₁₈ column, eluting with a gradient of MeCN/water containing 0.1% TFA, at flow rate of 60 mL/min) to obtain desired product. LC-MS calc. for C₂₂H₂₀F₆N₇ (M+H)⁺: m/z=496.2; found 496.2. ¹H NMR (500 MHz, DMSO-d₆) δ 9.12 (d, J=4.4 Hz, 1H), 9.06 (d, J=8.6 Hz, 1H), 8.83 (d, J=9.0 Hz, 1H), 7.97 (d, J=6.9 Hz, 1H), 7.75 (dd, J=8.7, 4.4 Hz, 1H), 7.21-7.16 (m, 2H), 6.99 (s, 1H), 4.24-4.16 (m, 1H), 4.04-3.95 (m, 1H), 2.34-2.27 (m, 1H), 2.09-1.17 (m, 7H) ppm.

### Example 12. (1S,3R)—N¹-(2-Bromo-5-(trifluoromethyl)pyrazolo[1,5-a]pyrimidin-7-yl)-N³-(imidazo[1,5-a]pyrazin-8-yl)cyclohexane-1,3-diamine

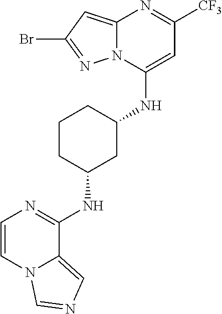 [SMILES: FC(F)(F)c1cc(N[C@H]2CCC[C@@H](Nc3nccn4cncc34)C2)n2nc(Br)cc2n1]

The title compound was prepared according to the procedure described in Example 8, using 8-chloroimidazo[1,5-a]pyrazine instead of 4-chloropyrazolo[1,5-a]pyrazine as a starting material. LC-MS calc. for C₁₉H₁₉BrF₃N₈(M+H)⁺: m/z=495.1; found 495.1.

### Example 13. (1S,3R)—N¹-(2-Bromo-5-(trifluoromethyl)pyrazolo[1,5-a]pyrimidin-7-yl)-N³-(3-methyl-3H-imidazo[4,5-c]pyridin-4-yl)cyclohexane-1,3-diamine

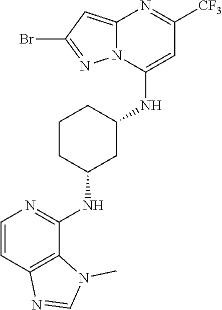 [SMILES: Cn1cnc2ccnc(N[C@@H]3CCC[C@H](Nc4cc(C(F)(F)F)nc5cc(Br)nn45)C3)c21]

The title compound was prepared according to the procedure described in Example 10, using 4-chloro-3-methyl-3H-imidazo[4,5-c]pyridine instead of 3-chloro-[1,2,4]triazolo[4,3-a]pyridine as a starting material. LC-MS calc. for C₂₀H₂₁BrF₃N₈(M+H)⁺: m/z=509.1; found 509.1.

### Example 14. (1S,3R)—N¹-(2,5-Bis(trifluoromethyl)pyrazolo[1,5-a]pyrimidin-7-yl)-N³-(imidazo[1,5-a]pyrazin-8-yl)cyclohexane-1,3-diamine

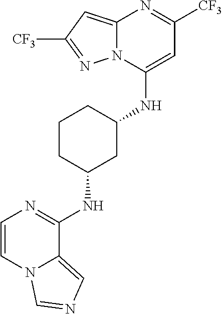 [SMILES: FC(F)(F)c1cc(N[C@H]2CCC[C@@H](Nc3nccn4cncc34)C2)n2nc(C(F)(F)F)cc2n1]

### Step 1. (1R,3S)—N¹-(Imidazo[1,5-a]pyrazin-8-yl)cyclohexane-1,3-diamine

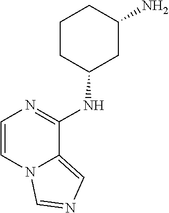 [SMILES: N[C@H]1CCC[C@@H](Nc2nccn3cncc23)C1]

The title compound was prepared according to the procedure described in Example 3, Step 1, using 8-chloroimidazo[1,5-a]pyrazine instead of 5-chloro-1,6-naphthyridine as a starting material. LCMS calc. for C₁₂H₁₈N₅(M+H)⁺: m/z=232.2; found: 232.2.

### Step 2. (1S,3R)—N¹-(2,5-Bis(trifluoromethyl)pyrazolo[1,5-a]pyrimidin-7-yl)-N³-(imidazo[1,5-a]pyrazin-8-yl)cyclohexane-1,3-diamine

The title compound was prepared according to the procedure described in Example 11, using (1R,3S)—N¹-(imidazo[1,5-a]pyrazin-8-yl)cyclohexane-1,3-diamine instead of (1R,3S)—N¹-(1,6-naphthyridin-5-yl)cyclohexane-1,3-diamine as a starting material. LC-MS calc. for C₂₀H₁₉F₆N₈ (M+H)⁺: m/z=485.2; found 485.2. ¹H NMR (500 MHz, DMSO-d₆) δ 8.79 (d, J=9.1 Hz, 1H), 8.26 (s, 1H), 7.79 (s, 1H), 7.60-7.56 (m, 1H), 7.44 (d, J=7.8 Hz, 1H), 7.16 (s, 1H), 7.03-6.98 (m, 2H), 4.34-4.25 (m, 1H), 4.17-4.06 (m, 1H), 2.26-2.19 (m, 1H), 2.05-1.19 (m, 7H) ppm.

### Example 15. (1S,3R)—N¹-(2,5-Bis(trifluoromethyl)pyrazolo[1,5-a]pyrimidin-7-yl)-N³-(3-methyl-3H-imidazo[4,5-c]pyridin-4-yl)cyclohexane-1,3-diamine

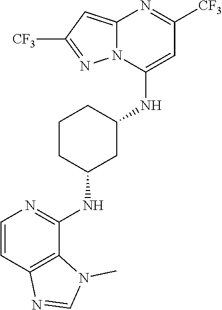 [SMILES: Cn1cnc2ccnc(N[C@@H]3CCC[C@H](Nc4cc(C(F)(F)F)nc5cc(C(F)(F)F)nn45)C3)c21]

The title compound was prepared according to the procedure described in Example 11, using (1R,3S)—N¹-(3-methyl-3H-imidazo[4,5-c]pyridin-4-yl)cyclohexane-1,3-diamine instead of (1R,3S)—N¹-(1,6-naphthyridin-5-yl)cyclohexane-1,3-diamine as a starting material. LC-MS calc. for C₂₁H₂₁F₆N₈ (M+H)⁺: m/z=499.2; found 499.2.

### Example 16. (1S,3R)—N¹-(2-Bromo-5-(trifluoromethyl)pyrazolo[1,5-a]pyrimidin-7-yl)-N³-(imidazo[1,2-c]pyrimidin-5-yl)cyclohexane-1,3-diamine

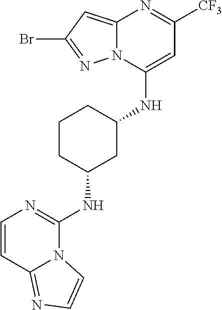 [SMILES: FC(F)(F)c1cc(N[C@H]2CCC[C@@H](Nc3nccc4nccn34)C2)n2nc(Br)cc2n1]

The title compound was prepared according to the procedure described in Example 10, using 5-chloroimidazo[1,2-c]pyrimidine instead of 3-chloro-[1,2,4]triazolo[4,3-a]pyridine as a starting material. LC-MS calc. for C₁₉H₁₉BrF₃N₈(M+H)⁺: m/z=495.1; found 495.2.

### Example 17. (1S,3R)—N¹-(2,5-Bis(trifluoromethyl)pyrazolo[1,5-a]pyrimidin-7-yl)-N³-(imidazo[1,2-c]pyrimidin-5-yl)cyclohexane-1,3-diamine

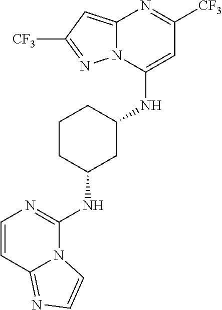 [SMILES: FC(F)(F)c1cc(N[C@H]2CCC[C@@H](Nc3nccc4nccn34)C2)n2nc(C(F)(F)F)cc2n1]

### Step 1. (1S,3R)—N¹-(2,5-Bis(trifluoromethyl)pyrazolo[1,5-a]pyrimidin-7-yl)cyclohexane-1,3-diamine

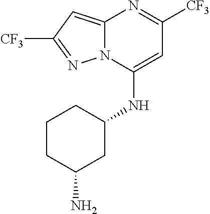 [SMILES: N[C@@H]1CCC[C@H](Nc2cc(C(F)(F)F)nc3cc(C(F)(F)F)nn23)C1]

To a solution of 2,5-bis(trifluoromethyl)pyrazolo[1,5-a]pyrimidin-7-ol (300.0 mg, 1.107 mmol) and DIPEA (0.58 mL, 3.32 mmol) in DCM (9 mL) was added trifluoromethanesulfonic anhydride solution (1.0 M in DCM, 1.66 mL, 1.66 mmol) and the resulting solution was stirred at r.t. for 10 min. A solution of tert-butyl ((1R,3S)-3-aminocyclohexyl)carbamate (356 mg, 1.66 mmol) in DCM (8 mL) was then added and the resulting solution was stirred at r.t. for 20 h. The mixture was diluted with DCM and water. The organic layer was separated and the crude product was purified by FCC (EtOAc/Hex) to obtain tert-butyl ((1R,3S)-3-((2,5-bis(trifluoromethyl)pyrazolo[1,5-a]pyrimidin-7-yl)amino)cyclohexyl)carbamate. The product was then taken up in DCM (5 mL) and TFA (3 mL), and the mixture was stirred at r.t. for 1 h. Solvent was removed the crude product was then purified by FCC (MeOH/DCM) to obtain the sub-title compound (148 mg, 1.107 mmol, 36.4% yield). LCMS calc. for C₁₄H₁₆F₆N₅ (M+H)⁺: m/z=368.1; found: 368.1.

### Step 2. (1S,3R)—N¹-(2,5-Bis(trifluoromethyl)pyrazolo[1,5-a]pyrimidin-7-yl)-N³-(imidazo[1,2-c]pyrimidin-5-yl)cyclohexane-1,3-diamine

A mixture of (1S,3R)—N¹-(2,5-bis(trifluoromethyl)pyrazolo[1,5-a]pyrimidin-7-yl)cyclohexane-1,3-diamine (20.0 mg, 0.054 mmol), 5-chloroimidazo[1,2-c]pyrimidine (16.7 mg, 0.109 mmol), and DIPEA (0.095 mL, 0.545 mmol) in DMSO (1 mL) was heated at 100° C. for 20 h. The mixture was then allowed to cool to r.t. and purified by prep.-LCMS (XBRIDGE® C₁₈ column, eluting with a gradient of MeCN/water containing 0.1% TFA, at flow rate of 60 mL/min) to obtain the title compound. LCMS calc. for C₂₀H₁₉F₆N₈ (M+H)⁺: m/z=485.2; found: 485.2.

### Example 18. (1R,3S)—N¹—([1,2,4]Triazolo[4,3-a]pyridin-3-yl)-N³-(2,5-bis(trifluoromethyl)pyrazolo[1,5-a]pyrimidin-7-yl)cyclohexane-1,3-diamine

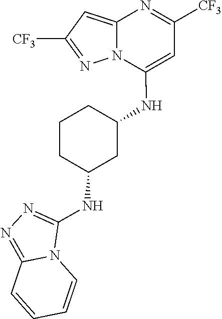 [SMILES: FC(F)(F)c1cc(N[C@H]2CCC[C@@H](Nc3nnc4ccccn34)C2)n2nc(C(F)(F)F)cc2n1]

The title compound was prepared according to the procedure described in Example 10, using (1S,3R)—N¹-(2,5-bis(trifluoromethyl)pyrazolo[1,5-a]pyrimidin-7-yl)cyclohexane-1,3-diamine instead of (1S,3R)—N¹-(2-bromo-5-(trifluoromethyl)pyrazolo[1,5-a]pyrimidin-7-yl)cyclohexane-1,3-diamine as a starting material. LC-MS calc. for C₂₀H₁₉F₆N₈ (M+H)⁺: m/z=485.2; found 485.2.

### Example A. MRGPRX2 FLIPR Calcium Mobilization Assay

Chinese hamster ovary (CHO-K1) cells stably expressing human MRGPRX2 were purchased from Genescript (Piscataway, NJ). The cells were maintained in culture medium (Ham's F-12K) containing 10% (v/v) FBS, 200 μg/mL Zeocin, 100 units/mL penicillin G and 100 μg/mL streptomycin (Life Technologies, Carlsbad, CA). For the assay, the cells were harvested and resuspended with culture medium without Zeocin before plating at 8000 cells per well in 20 μL in 384-well black clear bottom cell culture plates (VWR, Radnor, PA). After 24 h culture at 37° C. and 5% CO₂, the cells were loaded with 20 μL/well of calcium dye (FLIPR Calcium 6 Assay Kit, Molecular Devices, San Jose, CA) diluted in loading buffer (1× Hank's Balanced Salt Solution (HBSS), 5 mM probenecid, 20 mM 4-(2-hydroxyethyl)-1-piperazineethanesulfonic acid (HEPES), pH 7.4, VWR, Radnor, PA) followed with 45 min. incubation at 37° C. and 40 min. at r.t. in the dark. Cells were then treated with the addition of 10 uL/well test compounds with a range of increasing concentrations diluted in the assay buffer (1×HBSS, 20 mM HEPES, pH 7.4, VWR, Radnor, PA) with FLIPR Penta (Molecular Devices. San Jose, CA) and incubated for 30 min. at r.t. Ca²⁺ release was measured with FLIPR Penta with 10 s basal fluorescence measurement, then 12.5 μL of 5× agonist Cortistatin-14 (Tocris, Minneapolis, MN) at final concentration corresponding to the EC80 were added followed with continued fluorescence signal monitoring for an additional 110 s. The base line adjusted (median of first 10s base line) max value of the Relative Fluorescence Unit (RFU) was plotted against compound concentrations. Wells with no compound were served as the positive controls, and wells with high concentration of reference antagonist were used as negative controls. IC₅₀ curves were globally fitted with 3- or 4-parameter Hill equation in a Genedata Screener (Genedata Basel, Switzerland).

### Example B: MRGPRX2 IP1 HTRF Assay

This assay measures compound inhibition of myo-Inositol 1 phosphate (IP1) accumulation in CHO-K1 cells. Chinese hamster ovary (CHO-K1) cells stably expressing human MRGPRX2 were purchased from GenScript (Piscataway, NJ).

For the HTRF IP1 determination, an IP-One GqKit (Perkin Elmer Cisbio, Waltham, MA), which includes all reagents and buffers in this protocol, was used. The cells were maintained in culture medium (Ham's F-12K) containing 10% (v/v) FBS, and 200 μg/mL Zeocin. For the assay, the cells were harvested and resuspended in culture medium with 2% FBS and without Zeocin, then were passed through a 40 μm filter. Cells were added at 20000 cells in a 5 μL/well to a 384-well white small volume cell culture plate (Greiner VWR, Radnor, PA) which contained 50 nL/well of test compound serially diluted at a selected concentration range in DMSO. 5 μL/well of prepared 2× Stimulation Buffer2 was then added to plates and was incubated at 37° C. with 5% CO₂ for 1 h. 5 μL/well of agonist Cortistatin 14 (Bio-TechneR&D Systems, Minneapolis, MN) in 1×Stimulation Buffer 2, for a 1 μM final concentration, was then added to plates and incubated at 37° C. with 5% CO₂ for 1 h. 6 μL/well of 1:20 diluted Detection Reagent Mix (d2 and Cryptate) was added to the plates and incubated for 1 h in the dark. Plates were read on the PHERAstar microplate reader (BMG Labtech Cary, NC) to determine the HTRF ratio of the acceptor and donor emission signals. The IP1 concentrations were calculated from a standard curve following the IP-One GQ kit instructions. Wells with DMSO only and wells with high concentration of reference antagonist were used as controls for normalization. Compound IC₅₀ curves were globally fitted with 3- or 4-parameter Hill equation in a Genedata Screener (Genedata Basel, Switzerland).

### Example C: MRGPRX2 β-Arrestin Assay

This assay measures compound inhibition of β-Arrestin recruitment, which is part of the G protein-independent pathway that results from the ligand-activated GPCR phosphorylation by specific GPCR kinases. The PathHunter CHO-K1 MRPGRX2 B-Arrestin Cell line stably expressing ProLink tagged MRGPRX2 and Enzyme Acceptor Tagged B-Arrestin was purchased from Eurofins DiscoverX, (Fremont, CA).

The cells were maintained in culture medium from the Europhins DiscoverX Cell Culture Kit-107 which includes FBS, hygromycin B and G418. For the assay, the cells were harvested and were resuspended with Cell Plating Reagent 2 (Eurofins DiscoverX). Cells were added 10000 cells in 25 μL/well to a 384-well black cell culture plate (Greiner VWR, Radnor, PA) which contained 125 nL/well of compound at a selected serially diluted concentration range or DMSO. The plate was incubated at 37° C. with 5% CO₂ for 1 h. 2 μL/well of agonist Cortistatin 14 was diluted in Protein Dilution Buffer, (Eurofins DiscoverX) for a final concentration of 0.25 μM, was added and the plate was incubated at 37° C. with 5% CO₂ for 90 min. 14 μL/well of Detection Reagent Mix (PathHunter Detection Kit, Eurofins DiscoverX) was added to the plates and further incubated for 1 h in the dark. Plates were read on the PHERAstar microplate reader (BMG Labtech Cary, NC) measuring luminescence 0.1 to 1 s per well. Data was normalized using DMSO only wells and wells with high concentration of reference antagonist as controls. Compound IC₅₀ curves were globally fitted with 3- or 4-parameter Hill equation in a Genedata Screener (Genedata Basel, Switzerland).

### Table of Assay Data for the Assays of Examples A-C

Data obtained for the compounds of the Examples in the assays of Examples A (MRGPRX2 FLIPR), B (“IP1 HTRF”) and C (“MRGPRX2 β-Arrestin”) are shown in Table A. The symbol “++++” indicates an IC₅₀ value of ≤0.1 nM; “+++” indicates an IC₅₀ value of >0.1 nM but ≤100 nM; the symbol “++” indicates an IC₅₀ value of >100 nM but ≤1000 nM; the symbol “+” indicates an IC₅₀ value of >1000 nM. ND indicates no data.

TABLE A

| | | | MRGPRX2 |
|---|---|---|---|
| Example | MRGPRX2 FLIPR | MRGPRX2 IP1 HTRF | β-Arrestin |
| 1 | ++ | ++ | ND |
| 2 | +++ | ++ | ND |
| 3 | +++ | +++ | +++ |
| 4 | +++ | ++ | ND |
| 5 | +++ | +++ | +++ |
| 6 | ++ | ND | ND |
| 7 | +++ | ++ | ND |
| 8 | +++ | ++ | ND |
| 9 | +++ | +++ | ND |
| 10 | +++ | ++ | ND |
| 11 | +++ | +++ | ND |
| 12 | +++ | ++ | ND |
| 13 | +++ | ++ | ND |
| 14 | +++ | ++ | ND |
| 15 | +++ | ++ | ND |
| 16 | +++ | ++ | ND |
| 17 | +++ | +++ | ND |
| 18 | +++ | ++ | ND |

### Example D: Caco2 Assay

Caco-2 cells are grown at 37° C. in an atmosphere of 500 CO₂ in DMEM growth medium supplemented with 1000 (v/v) fetal bovine serum, 10% (v/v) nonessential amino acids, penicillin (100 U/mL), and streptomycin (100 μg/mL). Confluent cell monolayers are subcultured every 7 days or 4 days for Caco-2 by treatment with 0.050% trypsin containing 1 M EDTA. Caco-2 cells are seeded in 96-well Transwell plates. The seeding density for Caco-2 cells is 14,000 cells/well. DMEM growth medium is replaced every other day after seeding. Cell monolayers are used for transport assays between 22 and 25 days for Caco-2 cells.

Cell culture medium is removed and replaced with HBSS. To measure the TEER, the HBSS is added into the donor compartment (apical side) and receiver compartment (basolateral side). The TEER is measured by using a REMS Autosampler to ensure the integrity of the cell monolayers. Caco-2 cell monolayers with TEER values ≥300 Ω·cm² are used for transport experiments. To determine the Pₐₚₚ in the absorptive direction (A-B), solution of test compound (50 μM) in HBSS is added to the donor compartment (apical side), while HBSS solution with 40% BSA is added to the receiver compartment (basolateral side). The apical volume was 0.075 mL, and the basolateral volume is 0.25 mL. The incubation period is 120 min. at 37° C. in an atmosphere of 500 CO₂. At the end of the incubation period, samples from the donor and receiver sides are removed and an equal volume of MeCN is added for protein precipitation. The supernatants are collected after centrifugation (3000 rpm, Allegra X-14R Centrifuge from Beckman Coulter, Indianapolis, IN) for LCMS analysis. The permeability value is determined according to the equation:

$${P}_{app} ( cm / s ) = ( F * VD ) / SA * MD ) ,$$

where the flux rate (F, mass/time) is calculated from the slope of cumulative amounts of compound of interest on the receiver side, SA is the surface area of the cell membrane, VD is the donor volume, and MD is the initial amount of the solution in the donor chamber.

### Example E: Human Whole Blood Stability

The whole blood stability of the exemplified compounds is determined by LC-MS/MS.

The 96-Well Flexi-Tier™ Block (Analytical Sales & Services, Inc, Flanders, NJ) is used for the incubation plate containing 1.0 mL glass vials with 0.5 mL of blood per vial (pooled gender, human whole blood sourced from BIOIVT, Hicksville, NY or similar). Blood is pre-warmed in water bath to 37° C. for 30 min. 96-deep well analysis plate is prepared with the addition of 100 μL ultrapure water/well. 50 μL chilled ultrapure water/well is added to 96-deep well sample collection plate and covered with a sealing mat. 1 μL of 0.5 mM compound working solution (DMSO:water) is added to the blood in incubation plate to reach final concentrations of 1 μM, mixed by pipetting thoroughly and 50 μL is transferred 50 into the T=0 wells of the sample collection plate. Blood is allowed to sit in the water for 2 min. and then 400 μL stop solution/well is added (MeCN containing an internal standard). The incubation plate is placed in the Incu-Shaker CO₂ Mini incubator (Benchmark Scientific, Sayreville, NJ) at 37° C. with shaking at 150 rpm. At 1, 2 and 4-h, the blood samples are mixed thoroughly by pipetting and 50 μL is transferred into the corresponding wells of the sample collection plate. Blood is allowed to sit in the water for 2 min. and then 400 μL of stop solution/well is added. The collection plate is sealed and vortexed at 1700 rpm for 3 min. (VX-2500 Multi-Tube Vortexer, VWR International, Radnor, PA), and samples are then centrifuged in the collection plate at 3500 rpm for 10 min. (Allegra X-14R Centrifuge Beckman Coulter, Indianapolis, IN). 100 μL of supernatant/well is transferred from the sample collection plate into the corresponding wells of the analysis plate. The final plate is vortexed at 1700 rpm for 1 min. and analyze samples by LC-MS/MS. The peak area ratio of the 1, 2, and 4 h samples relative to T=0 is used to determine the percent remaining. The natural log of the percent remaining versus time is used determine a slope to calculate the compounds half-life in blood (t_{1/2}=0.693/slope).

### Example F: In Vitro Intrinsic Clearance Protocol

For in vitro metabolic stability experiments, test compounds are incubated with human liver microsomes at 37° C. The incubation mixture contains test compounds (1 M), NADPH (2 mM), and human liver microsomes (0.5 mg protein/mL) in 100 mM phosphate buffer (pH 7.4). The mixture is pre-incubated for 2 min. at 37° C. before the addition of NADPH. Reactions are commenced upon the addition of NADPH and quenched with ice-cold MeOH at 0, 10, 20, and 30 min. Terminated incubation mixtures are analyzed using LC-MS/MS system. The analytical system consisted of a Shimadzu LC-30AD binary pump system and SIL-30AC autosampler (Shimadzu Scientific Instruments, Columbia, MD) coupled with a Sciex Triple Quad 6500+ mass spectrometer from Applied Biosystems (Foster City, CA). Chromatographic separation of test compounds and internal standard is achieved using a Hypersil Gold C₁₈ column (50×2.1 mm, 5 μM, 175 Å) from ThermoFisher Scientific (Waltham, MA). Mobile phase A consists of 0.1% formic acid in water, and mobile phase B consists of 0.1% formic acid in MeCN. The total LC-MS/MS runtime can be 2.75 min. with a flow rate of 0.75 mL/min. Peak area integrations and peak area ratio calculations are performed using Analyst software (version 1.6.3) from Applied Biosystems.

The in vitro intrinsic clearance, CL_{int, in vitro}, is calculated from the t_{1/2} of test compound disappearance as CL_{int, in vitro}=(0.693/t_{1/2})λ(1/Cₚᵣₒₜₑᵢₙ), where Cₚᵣₒₜₑᵢₙ is the protein concentration during the incubation, and t_{1/2} is determined by the slope (k) of the log-linear regression analysis of the concentration versus time profiles; thus, t_{1/2}=ln 2/k. The CL_{int, in vitro} values are scaled to the in vivo values for human by using physiologically based scaling factors, hepatic microsomal protein concentrations (45 mg protein/g liver), and liver weights (21 g/kg body weight). The equation CLᵢₙₜ=CL_{int, in vitro}×(mg protein/g liver weight)×(g liver weight/kg body weight) is used. The in vivo hepatic clearance (CL_{H}) is then calculated by using CLᵢₙₜ and hepatic blood flow, Q (20 mL·min.⁻¹·kg⁻¹ in humans) in the well-stirred liver model disregarding all binding from CL_{H}=(Q×CLᵢₙₜ)/(Q+CLᵢₙₜ). The hepatic extraction ratio is calculated as CL_{H} divided by Q.

### Example G: In Vivo Pharmacokinetics Protocol

For in vivo pharmacokinetic experiments, test compounds are administered to male Sprague Dawley rats or male and female Cynomolgus monkeys intravenously or via oral gavage. For intravenous (IV) dosing, test compounds are dosed at 0.5 to 1 mg/kg using a formulation of 10% dimethylacetamide (DMAC) in acidified saline via IV bolus for rat and 5 min. or 10 min. IV infusion for monkey. For oral (PO) dosing, test compounds are dosed at 1.0 to 3.0 mg/kg using 5% DMAC in 0.5% methylcellulose in citrate buffer (pH 2.5). Blood samples are collected at predose and various time points up to 24 h postdose. All blood samples are collected using EDTA as the anticoagulant and centrifuged to obtain plasma samples. The plasma concentrations of test compounds are determined by LC-MS methods. The measured plasma concentrations are used to calculate PK parameters by standard noncompartmental methods using Phoenix® WinNonlin software program (version 8.0, Pharsight Corporation).

In rats and monkeys, cassette dosing of test compounds are conducted to obtain preliminary PK parameters.

In vivo pharmacokinetic experiments with male beagle dogs may be performed under the conditions described above.

### Example H: Time Dependent Inhibition (TDI) of CYP Protocol

This assay is designed to characterize an increase in CYP inhibition as a test compounds is metabolized over time. Potential mechanisms for this include the formation of a tight-binding, quasi-irreversible inhibitory metabolite complex or the inactivation of P450 enzymes by covalent adduct formation of metabolites. While this experiment employs a 10-fold dilution to diminish metabolite concentrations and therefore effects of reversible inhibition, it is possible (but not common) that a metabolite that is an extremely potent CYP inhibitor could result in a positive result.

The results are from a cocktail of CYP specific probe substrates at 4 times their Km concentrations for CYP2C9, 2C19, 2D6 and 3A4 (midazolam) using human liver microsomes (HLM). The HLMs can be pre-incubated with test compounds at a concentration 10 μM for 30 min. in the presence (+N) or absence (−N) of a NADPH regenerating system, diluted 10-fold, and incubated for 8 min. in the presence of the substrate cocktail with the addition of a fresh aliquot of NADPH regenerating system. A calibration curve of metabolite standards can be used to quantitatively measure the enzyme activity using LC-MS/MS. In addition, incubations with known time dependent inhibitors, tenellic acid (CYP2C9), ticlopidine (CYP2C19), paroxetine (CYP2D6), and troleandomycin (CYP3A4), used as positive controls are pre-incubated 30 min. with or without a NADPH regenerating system.

The analytical system consists of a Shimadzu LC-30AD binary pump system and SIL-30AC autosampler (Shimadzu Scientific Instruments, Columbia, MD) coupled with a Sciex Triple Quad 6500+ mass spectrometer from Applied Biosystems (Foster City, CA). Chromatographic separation of test compounds and internal standard can be achieved using an ACQUITY UPLC BEH 130A, 2.1×50 mm, 1.7 μm HPLC column (Waters Corp, Milford, MA). Mobile phase A consists of 0.1% formic acid in water, and mobile phase B consists of 0.1% formic acid in MeCN. The total LC-MS/MS runtime will be 2.50 min. with a flow rate of 0.9 mL/min. Peak area integrations and peak area ratio calculations are performed using Analyst software (version 1.6.3) from Applied Biosystems.

The percentage of control CYP2C9, CYP2C19, CYP2D6, and CYP3A4 activity remaining following preincubation of the compounds with NADPH is corrected for the corresponding control vehicle activity and then calculated based on 0 min. as 100%. A linear regression plot of the natural log of % activity remaining versus time for each isozyme is used to calculate the slope. The −slope is equal to the rate of enzyme loss, or the K_{obs}.

### Example I: In Vitro Degranulation Assay-β-Hexosaminidase Release Assay

The assay is performed essentially as described by Hermans, et al., Front. Immunol., 2021, 12, 625284. Mature mast cells are cultured in StemPro-34 medium with StemPro-34 nutrient supplement (Gibco), 2 mM L-glutamine (Gibco), 100 U/mL penicillin/100 μg/mL streptomycin (Gibco), 100 ng/mL recombinant human stem cell factor (PeproTech) and 100 ng/mL recombinant human interleukin 6 (Peprotech). The cells are washed in HEPES-buffered Tyrode's solution (Thermo Fisher) containing 0.1% (w/v) bovine serum albumine (further called “releasing medium”) and seeded at 1×10⁵ cells per well in 96-well plate in the releasing medium. Selected wells are pretreated with predefined concentrations of MRGPRX2 antagonist for 1 h at 37° C. with 5% CO₂. Then, about 10 μg/mL compound 48/80 (poly-p-methoxyphenethylmethylamine) or about 30 μM of substance P is added to treated wells and equal amount of releasing medium is added into negative control wells. Following 1 h incubation, the plate is centrifuged for 5 min. and 50 μL of supernatant is collected into a new 96-well plate. In a positive control group, remaining supernatant is removed and 100 μL 0.1% Triton X-100 (v/v) is added to lyse the cell at 300 g for 5 min. After that, the plate is centrifuged and 50 μL of the lysate is also transferred into the new plate. Supernatant and lysate samples are then incubated with 50 μL of p-nitrophenyl N-acetyle-B-D-glucosaminide in 0.1 M citrate buffer pH 4-4.5 (Thermo Fisher) for 1 h at 37° C. The enzymatic reaction is stopped by the addition of 100 μL/well of 0.1 M Na₂CO₃ buffer pH 10 (Thermo Fisher). The absorbance is read at 405 nm.

$$Net β - hexosaminidase release ( \% ) is calculated as = [ ( stimulated release - spontaneous release ) / total content in lysed cells ] \times 100 .$$

### Example J: Evaluation of the MRGPRX2 Antagonist Effects on In Vitro-Derived Human Mast Cells

This assay evaluates the effects of example compound on preventing mast cell degranulation via the MRGPRX2 receptor. The effects of example compounds on mast cell degranulation induced by Substance P (MRGPRX2 pathway), or IgE/anti-IgE is investigated by measuring β-hexosaminidase and mast+cell specific cytokine/chemokine release into the culture media. The addition of IgE/anti-IgE serves as a differentiation for the mechanism of action of the test compound.

Mature connective tissue-type mast cells (CTMCs) are plated at 50,000 cells by well in 50 μL and stimulated as follows.

### IgE Sensitization (Day 0)

All CTMCs are pre-treated with 100 μM IgE before treatment to coat the FcεR1 receptors with IgE to resemble in vitro mast cells. In this study, CTMCs are sensitized 48 h prior to pre-treatment with example compounds and degranulation (Day 2).

### Pre-Treatment (Day 0)

Omalizumab is added 4 h after IgE sensitization (IgE containing media is removed after 4 h and replaced with culture media containing omalizumab and kept for 48 h) and for the following 48 h before degranulation induction.

### Pre-Treatment (Day 2)

Test compounds and vehicles are added 48 h post IgE sensitization/omalizumab treatment for 30 min. at Day 2 of culture.

### Degranulation (Day 2+30 min.)

The CTMCs are stimulated with Substance P, Compound 48/80, anti-IgE, or Tyrode's buffer on Day 2 of culture.

### β-Hexosaminidase Assay to Assess Mast-Cell Degranulation

For the β-hexosaminidase assay, 50 μL of cell supernatant is collected in each well 45 min. after degranulation treatment. Supernatant is collected on n=3 wells per condition.

β-Hexosaminidase is a potent inflammatory mediator stored in mast cells and is released by activated mast cells. The determination of β-hexosaminidase is used to evaluate the level of mast cell degranulation. The assay is a colorimetric assay measuring the 4-nitrophenol production using a multimode plate reader. See J. Karhausen, et al., J. Clin. Invest., 2016, 126(10), 3981-98.

### Cytokine Release Assay

For the cytokine release assay, 60 μL of cell supernatant is collected in each well 8 h after degranulation treatment, an anti-protease cocktail added, and frozen at −80° C. until used. Supernatant is collected on n=3 wells per condition.

Cytokine release into the culture medium is measured using a custom V-PLEX Plus Human Cytokine Kit (MesoScale Discovery): TNF-α, IL-13, GM-CSF, VEGF-A, MCP-1, IL-6, IL-4, IL-5, IL-10, and IL-8. Culture media is diluted as follows:
Plate 1: Chemokine Panel 1 (human) kit—ref K15047 (panel MCP-1)—1:10;
Plate 2: Proinflammatory Panel 1 (human) kit—ref K15049 (panel IL-4, IL-6, IL-8, IL-10, IL-13, TNF-α)—1:5;
Plate 3: Cytokine Panel 1 (human) kit—ref K15050 (panel GM-CSF, IL-5, VEGF)—1:5.

### Example K: Determining the Effect of Example Compounds on Cytokine/Chemokine Release from HypoSkin Models After Subcutaneous Injection of Cetrorelix or Cortistatin-14

This assay evaluates the effect of the test compounds on cytokine/chemokine release (potentially from mast cells) upon systematic pre-treatment and treatment of HypoSkin models prior to subcutaneous injection of drugs known to degranulate mast cells and cause injection site reactions (namely, Cortistatin-14 and Cetrorelix).

74 HypoSkin models of 20 mm in diameter and 10 mm total thickness with 15/20 mm diameter silicon rings are produced from 2 donors (37 models per donor) according to standard procedures and cultured with 2 mL standard HypoSkin medium. Models are maintained in standard cell culture conditions for the whole culture duration at 37° C., 5% CO₂ and water saturation, with culture medium renewed every day except during weekends.

### Systematic Treatment

Vehicle control and test compounds are added to the culture media daily, from Day 0 to Day 2. The compounds remain in the culture media upon subcutaneous injections.

### Subcutaneous Injection

100 μL of Cetrorelix, Cortistatin-14, or PBS are subcutaneously injected in to the models on Day 2.

### End of Culture and Sampling

Culture media is collected on Days 2+8 h and Day 3 (24 h post-injection) and frozen at −80° C. Sampling of HypoSkin models is performed on Day 0 and Day 3 (24 h post-injection) and processed as follows:
½ are fixed in 10% buffered formalin and processed for paraffin wax embedding; and
½ are snap frozen and stored at −80° C.

### Hematoxylin and Eosin Staining

Hematoxylin and Eosin staining is performed on one 5 μm thick skin cross section for each sample. Representative images of both the epidermis/dermis and hypodermis are taken at 40× magnification to analyze skin structure integrity and viability.

### MSD Immunoassay

Cytokine release in the culture medium is measuring using the V-PLEX Plus Human Cytokine 36-Plex Kit (K15089G, MesoScale Discovery): Eotaxin, Eotaxin-3, GM-CSF, IFN-γ, IL-1α, IL-1β, Il-2, IL-4, IL-5, IL-6, IL-7, IL-8, IL-8 (HA), IL-10, IL12/IL-23p40, IL-23p70, IL-13, IL-15, IL-16, IL-17α, IL-21, IL-22, IL-23, IL-27, IL-31, IP-10, MCP-1, MCP-4, MDC, MIP-1α, MIP-1β, MIP-3α, TARC, TNF-α, TNF-β, and VEGF-A.

### Example L: Characterization of JAK Inhibitors MRGPRX2 Antagonists on Mast Cell Activation

The effect of the janus kinase inhibitors Povorcitinib, Ruxolitinib, and MRGPRX2 antagonists disclosed herein on the activation of mast cells in vitro is compared and characterized under different activating conditions, mimicking the in vivo disease situation. This assay determines the effect of JAK inhibition and MRGPRX2 antagonism on interference different mast cell activation pathways; the effect of JAK inhibition and MRGPRX2 antagonism in mast cells to prevent the release of mediators involved in the activation of primary human blood eosinophils and T cells.

Primary human skin mast cells (hsMC) are used to study how Povorcitinib, Ruxolitinib, and MRGPRX2 antagonists interfere with different prototype mast cell activators. The cells are obtained from individuals undergoing circumcision or breast reduction surgery by isolation from skin explants in a multi-step protocol. In vitro dose response studies using different concentration of the compounds to identify IC₅₀ values are conducted. For these studies, hsMCs are pre-incubated in the presence or absence of the compounds for 20 min, followed by stimulation with anti-IgE (FcεRI stimulation) or cortistatin-14 (MRGPRX2 agonist). Degranulation responses are measured by determination of β-hexosaminidase after 1 h at 37° C.

Next, three different concentrations of the compounds according to the determined IC₅₀ are employed to assess the spectrum of mast cell activating pathways inhibited by each compound. Therefore, hsMCs are pre-incubated in the presence or absence of the individual compounds for 20 min, followed by stimulation with five distinct activators: anti-IgE, corstatin-14 (CST), stem cell factor (SCF), complement peptides C3a, and C5a. In a parallel setup, hsMCs are pre-treated with various concentrations of cyclosporine A (CSA), to assess difference in efficacy to the test compounds. CSA, inhibiting the translocation of the transcription factor NFAT into the nucleus in MCs and other immune cells, is widely used to treat urticaria. As an alternative to CSA, another JAK or BTK inhibitor can be used. Degranulation responses are measured by determination of the β-hexosaminidase after 1 h at 37° C. In addition, cell viability and surface expression of FcεRI, MRGPRX2, cKitm C3aR/C5aR are measured by flow cytometry. Mast cells from three individual mast cell preparations are used.

To assess if treatment of mast cells with Povorcitinib, Ruxolitinib, or MRGPRX2 antagonists alters their mediator release profile and thereby their ability to activate eosinophils or T cells, hsMCs are pre-treated with the compounds for 20 min., followed by stimulation with anti-IgE, CST, SCF, C3a, or C5a for 1 h, 4 h, 8 h, and 24 h. Culture supernatants are collected and added to freshly isolated human peripheral blood eosinophils and total T cells. To achieve complete T cell activation, simultaneous activation of the TCR and costimulatory receptor with antibodies targeting CD3 and CD28 together with the mast cell supernatant is performed.

Cell activation is assessed using flow cytometry by measuring upregulation of CD69 and CD63 on eosinophils, as well as level of CD69, CD154, CD25, or CD62L on T cells. In addition, cytokines secreted by the mast cells are measured using bead-based cytokine multiplex assays (45 plex). Three individual experiments are performed using mast cells and eosinophils from three individual donors.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound having Formula (I): 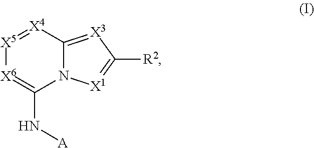
or a pharmaceutically acceptable salt thereof, wherein:
X¹ is N or CR¹;
X³ is N or CR³;
X⁴ is N or CR⁴;
X⁵ is N or CR⁵;
X⁶ is N or CR⁶;
R¹, R², R³, R⁴, R⁵, and R⁶ are each independently selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a1}SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R¹, R², R³, R⁴, R⁵, and R⁶ are each optionally substituted with 1, 2, 3, or 4 substituents each independently selected from R^{10A}; and wherein the C₁₋₆ alkyl, C₂₋₆ alkenyl, and C₂₋₆ alkynyl forming R¹, R², R³, R⁴, R⁵, and R⁶ are each optionally substituted with 1, 2, 3, or 4 substituents each independently selected from R^{10B};
A is a group of the Formula (A-1) or (A-2): 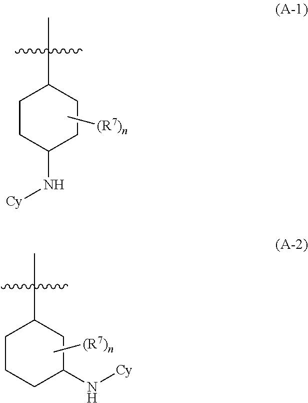
wherein [symbol] represents the point of attachment of A to the remainder of the molecule;
Cy is a ring selected from 5-10 membered heteroaryl and 5-10 membered heterocycloalkyl; and wherein the 5-10 membered heteroaryl and 5-10 membered heterocycloalkyl forming Cy are each optionally substituted with 1, 2, 3, 4, or 5 substituents independently selected from R^{Cy};
each R^{Cy} is independently selected from D, C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{Cy} are each optionally substituted with 1, 2, 3, or 4 substituents independently selected from R^{Cy1}; and wherein the C₁₋₆ alkyl, C₂₋₆ alkenyl, and C₂₋₆ alkynyl forming R^{Cy} are each optionally substituted with 1, 2, 3, or 4 substituents independently selected from R^{Cy2};
each R^{Cy1} is independently selected from C₁₋₆ alkyl, C₂₋₆ alkenyl, C₂₋₆ alkynyl, C₁₋₆ haloalkyl, and R^{Cy2}; wherein the C₁₋₆ alkyl, C₂₋₆ alkenyl, and C₂₋₆ alkynyl forming R^{Cy1} are each optionally substituted with 1, 2, 3, or 4 substituents independently selected from R^{Cy2};
each R^{Cy2} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a3}, SR^{a3}, C(O)R^{b3}, C(O)NR^{c3}R^{d3}, C(O)OR^{a3}, NR^{c3}R^{d3}, NR^{c3}C(O)R^{b3}, NR^{c3}C(O)OR^{a3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{Cy2} are each optionally substituted with 1, 2, 3, or 4 substituents independently selected from R^{g1};
R⁷ is selected from D, C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NOR^{a4})R^{b4}, C(═NR^{e4})NR^{c4}R^{d4}, NR^{c4}C(═NR^{e4})NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R⁷ are each optionally substituted with 1, 2, 3, or 4 substituents independently selected from R^{20A}; and wherein the C₁₋₆ alkyl, C₂₋₆ alkenyl, and C₂₋₆ alkynyl forming R⁷ are each optionally substituted with 1, 2, 3, or 4 substituents independently selected from R^{20B};
n is an integer from 0 to 8;
each R^{10A} is independently selected from C₁₋₆ alkyl, C₂₋₆ alkenyl, C₂₋₆ alkynyl, C₁₋₆ haloalkyl, and R^{10B}; wherein the C₁₋₆ alkyl, C₂₋₆ alkenyl, and C₂₋₆ alkynyl forming R^{10A} are each optionally substituted with 1, 2, 3, or 4 substituents independently selected from R^{10B};
each R^{10B} is independently selected from C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NOR^{a5})R^{b5}, C(═NR^{e5})NR^{c5}R^{d5}, NR^{c5}C(═NR^{e5})NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{10B} are each optionally substituted with 1, 2, 3, or 4 substituents independently selected from R¹¹;
each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a6}, SR^{a6}, C(O)R^{b6}, C(O)NR^{c6}R^{d6}, C(O)OR^{a6}, NR^{c6}R^{d6}, NR^{c6}C(O)R^{b6}, NR^{c6}C(O)OR^{a6}, NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6}, S(O)₂R^{b6}, and S(O)₂NR^{c6}R^{d6}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R¹¹ are each optionally substituted with 1, 2, 3, or 4 substituents independently selected from R^{g1}; and wherein the C₁₋₆ alkyl, C₂₋₆ alkenyl, and C₂₋₆ alkynyl forming R¹¹ are each optionally substituted with 1, 2, 3, or 4 substituents independently selected from R^{g2};
each R^{20A} is independently selected from C₁₋₆ alkyl, C₂₋₆ alkenyl, C₂₋₆ alkynyl, and C₁₋₆ haloalkyl; wherein the C₁₋₆ alkyl, C₂₋₆ alkenyl, and C₂₋₆ alkynyl forming R^{20A} are each optionally substituted with 1, 2, 3, or 4 substituents independently selected from R^{20B};
each R^{20B} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a7}, SR^{a7}, C(O)R^{b7}, C(O)NR^{c7}R^{d7}, C(O)OR^{a7}, NR^{c7}R^{d7}, NR^{c7}C(O)R^{b7}, NR^{c7}C(O)OR^{a7}, NR^{c7}S(O)R^{b7}, NR^{c7}S(O)₂R^{b7}, NR^{c7}S(O)₂NR^{c7}R^{d7}, S(O)R^{b7}, S(O)NR^{c7}R^{d7}, S(O)₂R^{b7}, and S(O)₂NR^{c7}R^{d7}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{20B} are each optionally substituted with 1, 2, 3, or 4 substituents independently selected from R^{g1};
each R^{a1}, R^{c1}, R^{d1}, R^{a2}, R^{c2}, R^{d2}, R^{a3}, R^{c3}, R^{d3}, R^{a4}, R^{c4}, R^{d4}, R^{a5}, R^{c5}, R^{d5}, R^{a6}, R^{c6}, R^{d6}, R^{a7}, R^{c7}, and R^{d7} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a1}, R^{c1}, R^{d1}, R^{a2}, R^{c2}, R^{d2}, R^{a3}, R^{c3}, R^{d3}, R^{a4}, R^{c4}, R^{d4}, R^{a5}, R^{c5}, R^{d5}, R^{a6}, R^{c6}, R^{d6}, R^{a7}, R^{c7}, and R^{d7} are each optionally substituted with 1, 2, 3, or 4 substituents independently selected from R^{g1}; and wherein the C₁₋₆ alkyl, C₂₋₆ alkenyl, and C₂₋₆ alkynyl forming R^{a1}, R^{c1}, R^{d1}, R^{a2}, R^{c2}, R^{d2}, R^{a3}, R^{c3}, R^{d3}, R^{a4}, R^{c4}, R^{d4}, R^{a5}, R^{c5}, R^{d5}, R^{a6}, R^{c6}, R^{d6}, R^{a7}, R^{c7}, and R^{d7} are each optionally substituted with 1, 2, 3, or 4 substituents independently selected from R^{g2};
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c6} and R^{d6} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c7} and R^{d7} attached to the same N atom, together with the N atom to which they are attached, form a 4-, 5-, 6- or 7-membered heterocycloalkyl group optionally substituted with 1, 2, 3, or 4 substituents independently selected from R^{g1};
each R^{b1}, R^{b2}, R^{b3}, R^{b4}, R^{b5}, R^{b6}, and R^{b7} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b1}, R^{b2}, R^{b3}, R^{b4}, R^{b5}, R^{b6}, and R^{b7} are each optionally substituted with 1, 2, 3, or 4 substituents independently selected from R^{g1}; and wherein the C₁₋₆ alkyl, C₂₋₆ alkenyl, and C₂₋₆ alkynyl forming R^{b1}, R^{b2}, R^{b3}, R^{b4}, R^{b5}, R^{b6}, and R^{b7} are each optionally substituted with 1, 2, 3, or 4 substituents independently selected from R^{g2};
each R^{e1}, R^{e2}, R^{e4}, and R^{e5} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{g1} is independently selected from C₁₋₆ alkyl, C₂₋₆ alkenyl, C₂₋₆ alkynyl, and R^{g2}, wherein the C₁₋₆ alkyl, C₂₋₆ alkenyl, and C₂₋₆ alkynyl are each optionally substituted with 1, 2, or 3 substituents independently selected from R^{g2};
each R^{g2} is independently selected from C₃₋₁₀ cycloalkyl, 4-10 membered heterocycloalkyl, C₆₋₁₀ aryl, 5-10 membered heteroaryl, halo, CN, OR^{a8}, SR^{a8}, C(O)R^{b8}, C(O)NR^{c8}R^{d8}, C(O)OR^{a8}, NR^{c8}R^{d8}, NR^{c8}C(O)R^{b8}, NR^{c8}C(O)OR^{a8}, NR^{c8}S(O)R^{b8}, NR^{c8}S(O)₂R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8}; wherein the C₃₋₁₀ cycloalkyl, 4-10 membered heterocycloalkyl, C₆₋₁₀ aryl, and 5-10 membered heteroaryl forming R^{g2} are each optionally substituted with 1, 2, 3, or 4 substituents independently selected from R^{h1};
each R^{a8}, R^{c8}, and R^{d8} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a8}, R^{c8}, and R^{d8} are each optionally substituted with 1, 2, 3, or 4 substituents independently selected from R^{h1}; and wherein the C₁₋₆ alkyl, C₂₋₆ alkenyl, and C₂₋₆ alkynyl forming R^{a8}, R^{c8}, and R^{d8} are each optionally substituted with 1, 2, 3, or 4 substituents independently selected from R^{h2};
each R^{b8} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b8} are each optionally substituted with 1, 2, 3, or 4 substituents independently selected from R^{h1}; and wherein the C₁₋₆ alkyl, C₂₋₆ alkenyl, and C₂₋₆ alkynyl forming R^{b11} are each optionally substituted with 1, 2, 3, or 4 substituents independently selected from R^{h2};
each R^{h1} is independently selected from C₁₋₆ alkyl, C₂₋₆ alkenyl, C₂₋₆ alkynyl, and R^{g2}, wherein the C₁₋₆ alkyl, C₂₋₆ alkenyl, and C₂₋₆ alkynyl are each optionally substituted with 1, 2, or 3 substituents independently selected from R^{h2};
each R^{h2} is independently selected from D, OH, NO₂, CN, halo, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino;
wherein at each occurrence, a heterocycloalkyl group has at least one ring-forming carbon atom and 1, 2, 3, or 4 ring-forming heteroatoms independently selected from N, O, and S; the N and S ring-forming heteroatoms of the heterocycloalkyl group are optionally oxidized; and a ring-forming carbon atom of the heterocycloalkyl group is optionally substituted by oxo to form a carbonyl group; and
at each occurrence, a heteroaryl group has at least one ring-forming carbon atom and 1, 2, 3, or 4 ring-forming heteroatoms independently selected from N, O, and S; the N and S ring-forming heteroatoms of the heteroaryl group are optionally oxidized; and a ring-forming carbon atom of the heteroaryl group is optionally substituted by oxo to form a carbonyl group;
with the proviso that the compound is not one of the following compounds:
N¹-(5-Methyl[1,2,4]triazolo[1,5-a]pyrimidin-7-yl)-N⁴-2-pyridinyl-1,4-cyclohexanediamine;
N¹,N⁴-Bis(5,6-dimethyl[1,2,4]triazolo[1,5-a]pyrimidin-7-yl)-1,4-cyclohexanediamine;
N¹-[3-(2,6-Dichloro-4-methoxyphenyl)-2,5-dimethylpyrazolo[1,5-a]pyrimidin-7-yl]-N⁴-(tetrahydro-2H-pyran-4-yl)-1,4-cyclohexanediamine;
N¹-[3-(2,6-Dichloro-4-methoxyphenyl)-2,5-dimethylpyrazolo[1,5-a]pyrimidin-7-yl]-N³-(tetrahydro-2H-pyran-4-yl)-1,3-cyclohexanediamine;
N¹-[3-(2,6-Dichloro-4-methoxyphenyl)-2,5-dimethylpyrazolo[1,5-a]pyrimidin-7-yl]-N³-[1-(phenylmethyl)-4-piperidinyl]-1,3-cyclohexanediamine.

2. The compound or pharmaceutically acceptable salt thereof of claim 1, wherein the compound has Formula (II-A), (II-B), (II-C), (II-D), (II-E), or (II-F): 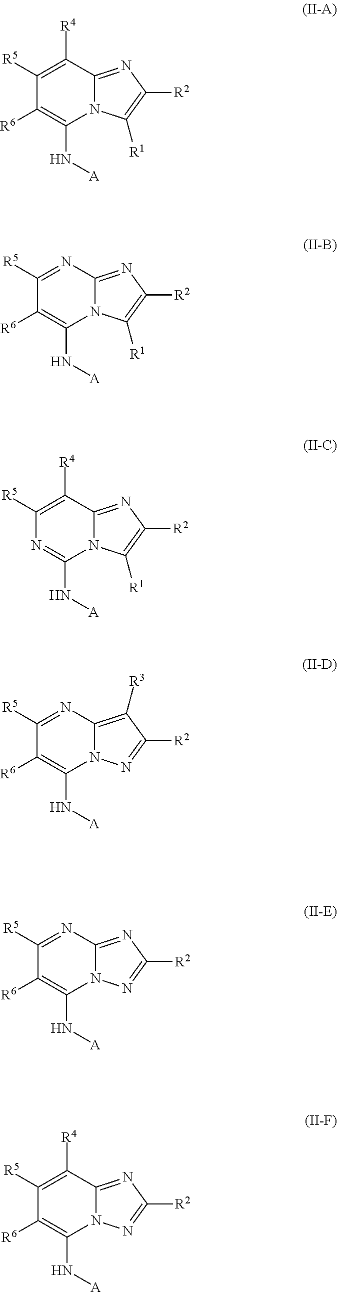
or a pharmaceutically acceptable salt thereof, wherein R¹, R², R³, R⁴, R⁵, R⁶, and A are as defined in claim 1.

3. The compound or pharmaceutically acceptable salt thereof of claim 1, wherein the compound is other than a compound of Formula (II-A); the compound is other than a compound of Formula (II-B); the compound is other than a compound of Formula (II-C); the compound is other than a compound of Formula (II-D); the compound is other than a compound of Formula (II-E); and/or the compound is other than a compound of Formula (II-F); wherein Formulae (II-A), (II-B), (II-C), (II-D), (II-E), and (II-F) are as defined in claim 2.

4. The compound or pharmaceutically acceptable salt thereof of claim 1, wherein A is a group of the Formula (A-1).

5. The compound or pharmaceutically acceptable salt thereof of claim 1, wherein A is a group of the Formula (A-3):

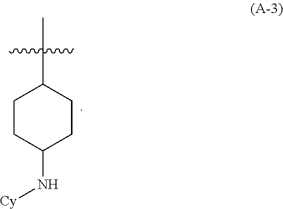

6. The compound or pharmaceutically acceptable salt thereof of claim 1, wherein A is a group of the Formula (A-4), (A-5), (A-6), or (A-7): 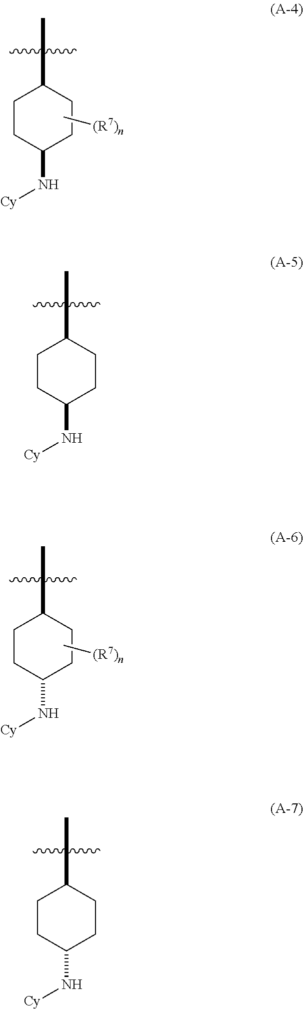
wherein the bold and dashed lines in the Formulae (A-4), (A-5), (A-6), and (A-7) indicate relative (i.e., cis or trans) stereochemistry.

7. The compound or pharmaceutically acceptable salt thereof of claim 1, wherein A is a group of the Formula (A-2).

8. The compound or pharmaceutically acceptable salt thereof of claim 1, wherein A is a group of the Formula (A-8):

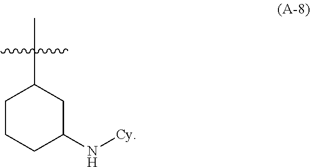

9. The compound or pharmaceutically acceptable salt thereof of claim 1, wherein A is a group of the Formula (A-9) or (A-10): 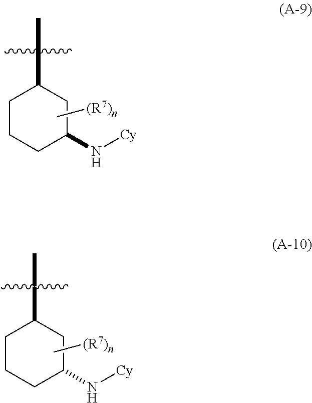
wherein the bold and dashed lines in the Formulae (A-7) and (A-8) indicate relative (i.e., cis or trans) stereochemistry.

10. The compound or pharmaceutically acceptable salt thereof of claim 1, wherein A is a group of the Formula (A-11), (A-12), (A-13) or (A-14): 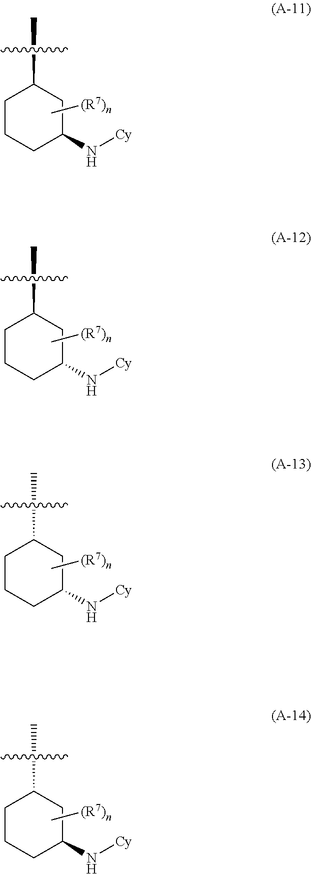
wherein the wedged lines in the Formulae (A-11), (A-12), (A-13) and (A-14) indicate absolute (i.e., (R) or (S)) stereochemistry.

11. The compound or pharmaceutically acceptable salt thereof of claim 1, wherein A is a group of the Formula (A-15) or (A-16): 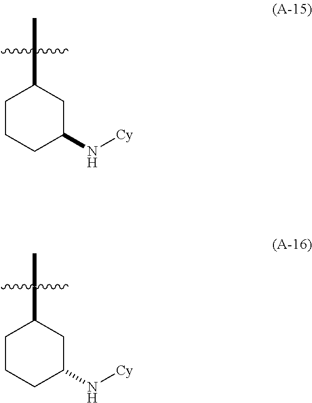
wherein the bold and dashed lines in the Formulae (A-15) and (A-16) indicate relative (i.e., cis or trans) stereochemistry.

12. The compound or pharmaceutically acceptable salt thereof of claim 1, wherein A is a group of the Formula (A-17), (A-18), (A-19) or (A-20): 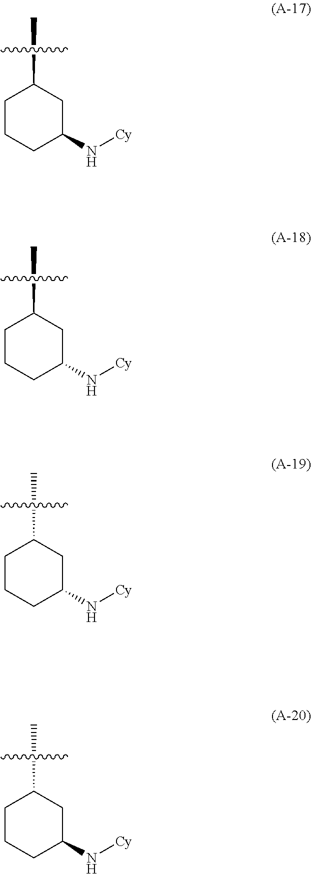
wherein the wedged lines in the Formulae (A-17), (A-18), (A-19) and (A-20) indicate absolute (i.e., (R) or (S)) stereochemistry.

13. The compound or pharmaceutically acceptable salt thereof of claim 1, wherein X¹ is CR¹.

14. The compound or pharmaceutically acceptable salt thereof of claim 1, wherein X¹ is N.

15. The compound or pharmaceutically acceptable salt thereof of claim 1, wherein R¹ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₁₋₆ alkyl, C₂₋₆ alkenyl, and C₂₋₆ alkynyl forming R² are each optionally substituted with 1, 2, 3, or 4 substituents each independently selected from R^{10B}.

16. The compound or pharmaceutically acceptable salt thereof of claim 1, wherein R¹ is selected from H, D, C₁₋₆ alkyl, C₁₋₆ haloalkyl, halo, CN, OR^{a1}; wherein the C₁₋₆ alkyl forming R¹ is optionally substituted with 1, 2, 3, or 4 substituents each independently selected from R^{10B}.

17. The compound or pharmaceutically acceptable salt thereof of claim 1, wherein R¹ is selected from H, D, C₁₋₆ alkyl (such as methyl or ethyl), C₁₋₆ haloalkyl (such as CF₃, CHF₂, CF₂CF₃), CN, or halo (such as F, Cl, or Br).

18. The compound or pharmaceutically acceptable salt thereof of claim 1, wherein R¹ is H.

19. The compound or pharmaceutically acceptable salt thereof of claim 1, wherein X² is CR².

20. The compound or pharmaceutically acceptable salt thereof of claim 1, wherein X² is N.

21. The compound or pharmaceutically acceptable salt thereof of claim 1, wherein R² is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₁₋₆ alkyl, C₂₋₆ alkenyl, and C₂₋₆ alkynyl forming R² are each optionally substituted with 1, 2, 3, or 4 substituents each independently selected from R^{10B}.

22. The compound or pharmaceutically acceptable salt thereof of claim 1, wherein R² is H, D, C₁₋₆ alkyl (such as methyl or ethyl), C₁₋₆ haloalkyl (such as CF₃, CHF₂, CF₂CF₃), CN, or halo (such as F, Cl, or Br).

23. The compound or pharmaceutically acceptable salt thereof of claim 1, wherein R² is C₁₋₆ alkyl, C₁₋₆ haloalkyl, or halo.

24. The compound or pharmaceutically acceptable salt thereof of claim 1, wherein R² is CF₃, CH₃, or Br.

25. The compound or pharmaceutically acceptable salt thereof of claim 1, wherein X³ is CR³.

26. The compound or pharmaceutically acceptable salt thereof of claim 1, wherein X³ is N.

27. The compound or pharmaceutically acceptable salt thereof of claim 1, wherein R³ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₁₋₆ alkyl, C₂₋₆ alkenyl, and C₂₋₆ alkynyl forming R³ are each optionally substituted with 1, 2, 3, or 4 substituents each independently selected from R^{10B}.

28. The compound or pharmaceutically acceptable salt thereof of claim 1, wherein R³ is selected from H, D, C₁₋₆ alkyl, C₁₋₆ haloalkyl, halo, CN, OR^{a1}; wherein the C₁₋₆ alkyl forming R³ is optionally substituted with 1, 2, 3, or 4 substituents each independently selected from R^{10B}.

29. The compound or pharmaceutically acceptable salt thereof of claim 1, wherein R³ is H, D, C₁₋₆ alkyl (such as methyl or ethyl), C₁₋₆ haloalkyl (such as CF₃, CHF₂, CF₂CF₃), CN, or halo (such as F, Cl, or Br).

30. The compound or pharmaceutically acceptable salt thereof of claim 1, wherein R³ is H.

31. The compound or pharmaceutically acceptable salt thereof of claim 1, wherein X⁴ is CR⁴.

32. The compound or pharmaceutically acceptable salt thereof of claim 1, wherein X⁴ is N.

33. The compound or pharmaceutically acceptable salt thereof of claim 1, wherein R⁴ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₁₋₆ alkyl, C₂₋₆ alkenyl, and C₂₋₆ alkynyl forming R⁴ are each optionally substituted with 1, 2, 3, or 4 substituents each independently selected from R^{10B}.

34. The compound or pharmaceutically acceptable salt thereof of claim 1, wherein R⁴ is selected from H, D, C₁₋₆ alkyl, C₁₋₆ haloalkyl, halo, CN, OR^{a1}; wherein the C₁₋₆ alkyl forming R⁴ is optionally substituted with 1, 2, 3, or 4 substituents each independently selected from R^{10B}.

35. The compound or pharmaceutically acceptable salt thereof of claim 1, wherein R⁴ is H, D, C₁₋₆ alkyl (such as methyl or ethyl), C₁₋₆ haloalkyl (such as CF₃, CHF₂, CF₂CF₃), CN, or halo (such as F, Cl, or Br).

36. The compound or pharmaceutically acceptable salt thereof of claim 1, wherein R⁴ is H.

37. The compound or pharmaceutically acceptable salt thereof of claim 1, wherein X⁵ is CR⁵.

38. The compound or pharmaceutically acceptable salt thereof of claim 1, wherein X⁵ is N.

39. The compound or pharmaceutically acceptable salt thereof of claim 1, wherein R⁵ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₁₋₆ alkyl, C₂₋₆ alkenyl, and C₂₋₆ alkynyl forming R⁵ are each optionally substituted with 1, 2, 3, or 4 substituents each independently selected from R^{10B}.

40. The compound or pharmaceutically acceptable salt thereof of claim 1, wherein R⁵ is H, D, C₁₋₆ alkyl (such as methyl or ethyl), C₁₋₆ haloalkyl (such as CF₃, CHF₂, CF₂CF₃), CN, or halo (such as F, Cl, or Br).

41. The compound or pharmaceutically acceptable salt thereof of claim 1, wherein R⁵ is H or C₁₋₆ haloalkyl.

42. The compound or pharmaceutically acceptable salt thereof of claim 1, wherein R⁵ is CF₃.

43. The compound or pharmaceutically acceptable salt thereof of claim 1, wherein R⁵ is H.

44. The compound or pharmaceutically acceptable salt thereof of claim 1, wherein X⁶ is CR⁶.

45. The compound or pharmaceutically acceptable salt thereof of claim 1, wherein X⁶ is N.

46. The compound or pharmaceutically acceptable salt thereof of claim 1, wherein R⁶ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₁₋₆ alkyl, C₂₋₆ alkenyl, and C₂₋₆ alkynyl forming R⁶ are each optionally substituted with 1, 2, 3, or 4 substituents each independently selected from R^{10B}.

47. The compound or pharmaceutically acceptable salt thereof of claim 1, wherein R⁶ is selected from H, D, C₁₋₆ alkyl, C₁₋₆ haloalkyl, halo, CN, OR^{a1}; wherein the C₁₋₆ alkyl forming R⁶ is optionally substituted with 1, 2, 3, or 4 substituents each independently selected from R^{10B}.

48. The compound or pharmaceutically acceptable salt thereof of claim 1, wherein R⁶ is H, D, C₁₋₆ alkyl (such as methyl or ethyl), C₁₋₆ haloalkyl (such as CF₃, CHF₂, CF₂CF₃), CN, or halo (such as F, Cl, or Br).

49. The compound or pharmaceutically acceptable salt thereof of claim 1, wherein R⁶ is H.

50. The compound or pharmaceutically acceptable salt thereof of claim 1, wherein R⁷ is selected from D, C₁₋₆ alkyl, C₂₋₆ alkenyl, C₂₋₆ alkynyl, C₁₋₆ haloalkyl, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NOR^{a4})R^{b4}, C(═NR^{e4})NR^{c4}R^{d4}, NR^{c4}C(═NR^{e4})NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein the C₁₋₆ alkyl, C₂₋₆ alkenyl, and C₂₋₆ alkynyl forming R⁷ are each optionally substituted with 1, 2, 3, or 4 substituents each independently selected from R^{20B}.

51. The compound or pharmaceutically acceptable salt thereof of claim 1, wherein R⁷ is D, C₁₋₆ alkyl (such as methyl or ethyl), C₁₋₆ haloalkyl (such as CF₃, CHF₂, CF₂CF₃), CN, or halo (such as F, Cl, or Br).

52. The compound or pharmaceutically acceptable salt thereof of claim 1, wherein Cy is optionally substituted with 1, 2, 3, 4, 5 or 6 substituents independently selected from R^{Cy}.

53. The compound or pharmaceutically acceptable salt thereof of claim 1, wherein Cy is optionally substituted with 1, 2, 3, 4, or 5 substituents independently selected from R^{Cy}.

54. The compound or pharmaceutically acceptable salt thereof of claim 1, wherein Cy is optionally substituted with 1, 2, 3, or 4 substituents independently selected from R^{Cy}.

55. The compound or pharmaceutically acceptable salt thereof of claim 1, wherein Cy is optionally substituted with 1, 2, or 3 substituents independently selected from R^{Cy}.

56. The compound or pharmaceutically acceptable salt thereof of claim 1, wherein Cy is optionally substituted with 1 or 2 substituents independently selected from R^{Cy}.

57. The compound or pharmaceutically acceptable salt thereof of claim 1, wherein Cy is optionally substituted with 1 substituent selected from R^{Cy}.

58. The compound or pharmaceutically acceptable salt thereof of claim 1, wherein Cy is unsubstituted.

59. The compound or pharmaceutically acceptable salt thereof of claim 1, wherein Cy is 5-10 membered heteroaryl optionally substituted with 1, 2, 3, 4, 5 or 6 substituents independently selected from R^{Cy}.

60. The compound or pharmaceutically acceptable salt thereof of claim 1, wherein Cy is 5-10 membered heteroaryl optionally substituted with 1, 2, 3, 4, or 5 substituents independently selected from R^{Cy}.

61. The compound or pharmaceutically acceptable salt thereof of claim 1, wherein Cy is 5-10 membered heteroaryl optionally substituted with 1, 2, 3, or 4 substituents independently selected from R^{Cy}.

62. The compound or pharmaceutically acceptable salt thereof of claim 1, wherein Cy is 5-10 membered heteroaryl optionally substituted with 1, 2, or 3 substituents independently selected from R^{Cy}.

63. The compound or pharmaceutically acceptable salt thereof of claim 1, wherein Cy is 5-10 membered heteroaryl optionally substituted with 1 or 2 substituents independently selected from R^{Cy}.

64. The compound or pharmaceutically acceptable salt thereof of claim 1, wherein Cy is 5-10 membered heteroaryl optionally substituted with 1 substituent selected from R^{Cy}.

65. The compound or pharmaceutically acceptable salt thereof of claim 1, wherein Cy is unsubstituted 5-10 membered heteroaryl.

66. The compound or pharmaceutically acceptable salt thereof of claim 1, wherein Cy is unsubstituted or substituted 3H-imidazo[4,5-c]pyridinyl, 1,6-naphthyridinyl, imidazo[1,5-a]pyrazinyl, pyrazolo[1,5-a]pyrazinyl, [1,2,4]triazolo[4,3-a]pyridinyl, or imidazo[1,2-c]pyrimidinyl.

67. The compound or pharmaceutically acceptable salt thereof of claim 1, wherein each R^{Cy} is independently selected from C₁₋₆ alkyl, C₂₋₆ alkenyl, C₂₋₆ alkynyl, C₁₋₆ haloalkyl, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR²C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein the C₁₋₆ alkyl, C₂₋₆ alkenyl, and C₂₋₆ alkynyl forming R^{Cy} are each optionally substituted with 1, 2, 3, or 4 substituents independently selected from R^{Cy2}.

68. The compound or pharmaceutically acceptable salt thereof of claim 1, wherein each R^{Cy} is selected from C₁₋₆ alkyl (such as methyl or ethyl), C₁₋₆ haloalkyl (such as CF₃, CHF₂, CF₂CF₃), halo (such as F, Cl, or Br), and OR^{a2} (such as methoxy or ethoxy).

69. The compound or pharmaceutically acceptable salt thereof of claim 1, wherein each R^{Cy} is selected from C₁₋₆ alkyl.

70. The compound or pharmaceutically acceptable salt thereof of claim 1, wherein R^{Cy} is CH₃.

71. The compound or pharmaceutically acceptable salt thereof of claim 1, wherein n is 0.

72. The compound or pharmaceutically acceptable salt thereof of claim 1, wherein n is an integer from 1 to 4.

73. The compound or pharmaceutically acceptable salt thereof of claim 1, wherein n is 1.

74. The compound or pharmaceutically acceptable salt thereof of claim 1, wherein:
X¹ is N or CR¹;
X³ is N or CR³;
X⁴ is N or CR⁴;
X⁵ is N or CR⁵;
X⁶ is N or CR⁶;
R¹, R², R³, R⁴, R⁵, and R⁶ are each independently selected from H, D, C₁₋₆ alkyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, NR^{c1}R^{d1}, and S(O)₂R^{b1};
A is a group of the Formula (A-1) or (A-2): 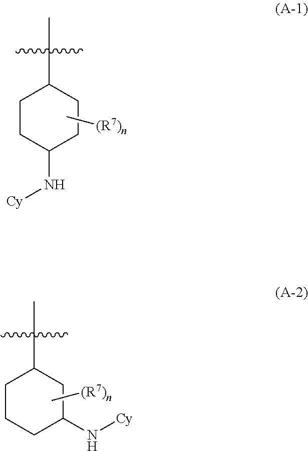
wherein [symbol] represents the point of attachment of A to the remainder of the molecule;
Cy is a ring selected from 5-10 membered heteroaryl optionally substituted with 1, 2, 3, 4, or 5 substituents independently selected from R^{Cy};
each R^{Cy} is independently selected from C₁₋₆ alkyl, C₃₋₁₀ cycloalkyl, 4-12 membered heterocycloalkyl, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R⁷ is selected from D, C₁₋₆ alkyl, C₁₋₆ haloalkyl, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NOR^{a4})R^{b4}, C(═NR^{e4})NR^{c4}R^{d4}, NR^{c4}C(═NR⁴)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};
n is an integer from 0 to 8;
each R^{a1}, R^{c1}, R^{d1}, R^{a2}, R^{c2}, R^{d2}, R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
each R^{b1}, R^{b2}, and R^{b4} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
each R^{e2} and R^{e4} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{g1} is independently selected from C₁₋₆ alkyl, C₂₋₆ alkenyl, C₂₋₆ alkynyl, and R^{g2}, wherein the C₁₋₆ alkyl, C₂₋₆ alkenyl, and C₂₋₆ alkynyl are each optionally substituted with 1, 2, or 3 substituents independently selected from R^{g2};
each R^{g2} is independently selected from C₃₋₁₀ cycloalkyl, 4-10 membered heterocycloalkyl, C₆₋₁₀ aryl, 5-10 membered heteroaryl, halo, CN, OR^{a8}, SR^{a8}, C(O)R^{b8}, C(O)NR^{c8}R^{d8}, C(O)OR^{a8}, NR^{c8}R^{d8}, NR^{c8}C(O)R^{b8}, NR^{c8}C(O)OR^{a8}, NR^{c8}S(O)R^{b8}, NR^{c8}S(O)₂R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8};
each R^{a8}, R^{c8}, and R^{d8} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
each R^{b8} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
wherein at each occurrence, a heterocycloalkyl group has at least one ring-forming carbon atom and 1, 2, 3, or 4 ring-forming heteroatoms independently selected from N, O, and S; the N and S ring-forming heteroatoms of the heterocycloalkyl group are optionally oxidized; and a ring-forming carbon atom of the heterocycloalkyl group is optionally substituted by oxo to form a carbonyl group; and
at each occurrence, a heteroaryl group has at least one ring-forming carbon atom and 1, 2, 3, or 4 ring-forming heteroatoms independently selected from N, O, and S; the N and S ring-forming heteroatoms of the heteroaryl group are optionally oxidized; and a ring-forming carbon atom of the heteroaryl group is optionally substituted by oxo to form a carbonyl group;
with the proviso that the compound is not one of the following compounds:
N¹-(5-Methyl[1,2,4]triazolo[1,5-a]pyrimidin-7-yl)-N⁴-2-pyridinyl-1,4-cyclohexanediamine;
N¹,N⁴-Bis(5,6-dimethyl[1,2,4]triazolo[1,5-a]pyrimidin-7-yl)-1,4-cyclohexanediamine;
N¹-[3-(2,6-Dichloro-4-methoxyphenyl)-2,5-dimethylpyrazolo[1,5-a]pyrimidin-7-yl]-N⁴-(tetrahydro-2H-pyran-4-yl)-1,4-cyclohexanediamine;
N¹-[3-(2,6-Dichloro-4-methoxyphenyl)-2,5-dimethylpyrazolo[1,5-a]pyrimidin-7-yl]-N³-(tetrahydro-2H-pyran-4-yl)-1,3-cyclohexanediamine;
N¹-[3-(2,6-Dichloro-4-methoxyphenyl)-2,5-dimethylpyrazolo[1,5-a]pyrimidin-7-yl]-N³-[1-(phenylmethyl)-4-piperidinyl]-1,3-cyclohexanediamine.

75. The compound or pharmaceutically acceptable salt thereof of claim 1, wherein the compound has Formula (IIA): 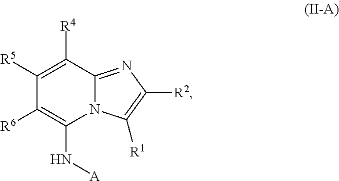
wherein:
R¹, R², R⁴, R⁵, and R⁶ are each independently selected from H, D, C₁₋₆ alkyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, NR^{c1}R^{d1}, and S(O)₂R^{b1};
A is a group of the Formula (A-4): 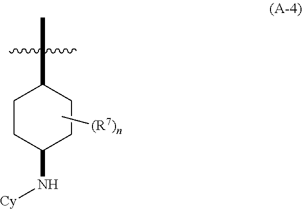
wherein [symbol] represents the point of attachment of A to the remainder of the molecule;
Cy is a ring selected from 5-10 membered heteroaryl optionally substituted with 1, 2, 3, 4, or 5 substituents independently selected from R^{Cy};
each R^{Cy} is independently selected from C₁₋₆ alkyl, C₃₋₁₀ cycloalkyl, 4-12 membered heterocycloalkyl, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R⁷ is selected from D, C₁₋₆ alkyl, C₁₋₆ haloalkyl, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NOR^{a4})R^{b4}, C(═NR^{e4})NR^{c4}R^{d4}, NR^{c4}C(═NR^{e4})NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};
n is an integer from 0 to 8;
each R^{a1}, R^{c1}, R^{d1}, R^{a2}, R^{c2}, R^{d2}, R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
each R^{b1}, R^{b2}, and R^{b4} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
each R^{e2} and R^{e4} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{g1} is independently selected from C₁₋₆ alkyl, C₂₋₆ alkenyl, C₂₋₆ alkynyl, and R^{g2}, wherein the C₁₋₆ alkyl, C₂₋₆ alkenyl, and C₂₋₆ alkynyl are each optionally substituted with 1, 2, or 3 substituents independently selected from R^{g2};
each R^{g2} is independently selected from C₃₋₁₀ cycloalkyl, 4-10 membered heterocycloalkyl, C₆₋₁₀ aryl, 5-10 membered heteroaryl, halo, CN, OR^{a8}, SR^{a8}, C(O)R^{b8}, C(O)NR^{c8}R^{d8}, C(O)OR^{a8}, NR^{c8}R^{d8}, NR^{c8}C(O)R^{b8}, NR^{c8}C(O)OR^{a8}, NR^{c8}S(O)R^{b8}, NR^{c8}S(O)₂R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8};
each R^{a8}, R^{c8}, and R^{d8} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
each R^{b8} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
wherein at each occurrence, a heterocycloalkyl group has at least one ring-forming carbon atom and 1, 2, 3, or 4 ring-forming heteroatoms independently selected from N, O, and S; the N and S ring-forming heteroatoms of the heterocycloalkyl group are optionally oxidized; and a ring-forming carbon atom of the heterocycloalkyl group is optionally substituted by oxo to form a carbonyl group; and
at each occurrence, a heteroaryl group has at least one ring-forming carbon atom and 1, 2, 3, or 4 ring-forming heteroatoms independently selected from N, O, and S; the N and S ring-forming heteroatoms of the heteroaryl group are optionally oxidized; and a ring-forming carbon atom of the heteroaryl group is optionally substituted by oxo to form a carbonyl group.

76. The compound or pharmaceutically acceptable salt thereof of claim 1, wherein the compound has Formula (II-A): 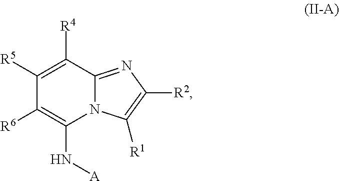
wherein:
R¹, R², R⁴, R⁵, and R⁶ are each independently selected from H, D, C₁₋₆ alkyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, NR^{c1}R^{d1}, and S(O)₂R^{b1};
A is a group of the Formula (A-9): 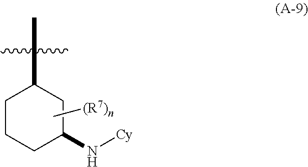
wherein [symbol] represents the point of attachment of A to the remainder of the molecule;
Cy is a ring selected from 5-10 membered heteroaryl optionally substituted with 1, 2, 3, 4, or 5 substituents independently selected from R^{Cy};
each R^{Cy} is independently selected from C₁₋₆ alkyl, C₃₋₁₀ cycloalkyl, 4-12 membered heterocycloalkyl, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R⁷ is selected from D, C₁₋₆ alkyl, C₁₋₆ haloalkyl, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NOR^{a4})R^{b4}, C(═NR^{e4})NR^{c4}R^{d4}, NR^{c4}C(═NR^{e4})NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};
n is an integer from 0 to 8;
each R^{a1}, R^{c1}, R^{d1}, R^{a2}, R^{c2}, R^{d2}, R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
each R^{b1}, R^{b2}, and R^{b4} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
each R^{e2} and R^{e4} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{g1} is independently selected from C₁₋₆ alkyl, C₂₋₆ alkenyl, C₂₋₆ alkynyl, and R^{g2}, wherein the C₁₋₆ alkyl, C₂₋₆ alkenyl, and C₂₋₆ alkynyl are each optionally substituted with 1, 2, or 3 substituents independently selected from R^{g2};
each R^{g2} is independently selected from C₃₋₁₀ cycloalkyl, 4-10 membered heterocycloalkyl, C₆₋₁₀ aryl, 5-10 membered heteroaryl, halo, CN, OR^{a8}, SR^{a8}, C(O)R^{b8}, C(O)NR^{c8}R^{d8}, C(O)OR^{a8}, NR^{c8}R^{d8}, NR^{c8}C(O)R^{b8}, NR^{c8}C(O)OR^{a8}, NR^{c8}S(O)R^{b8}, NR^{c8}S(O)₂R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8};
each R^{a8}, R^{c8}, and R^{d8} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
each R^{b8} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
wherein at each occurrence, a heterocycloalkyl group has at least one ring-forming carbon atom and 1, 2, 3, or 4 ring-forming heteroatoms independently selected from N, O, and S; the N and S ring-forming heteroatoms of the heterocycloalkyl group are optionally oxidized; and a ring-forming carbon atom of the heterocycloalkyl group is optionally substituted by oxo to form a carbonyl group; and
at each occurrence, a heteroaryl group has at least one ring-forming carbon atom and 1, 2, 3, or 4 ring-forming heteroatoms independently selected from N, O, and S; the N and S ring-forming heteroatoms of the heteroaryl group are optionally oxidized; and a ring-forming carbon atom of the heteroaryl group is optionally substituted by oxo to form a carbonyl group.

77. The compound or pharmaceutically acceptable salt thereof of claim 1, wherein the compound has Formula (II-D): 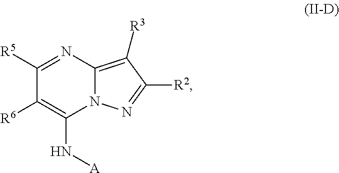
wherein:
R², R³, R⁵, and R⁶ are each independently selected from H, D, C₁₋₆ alkyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, NR^{c1}R^{d1}, and S(O)₂R^{b1};
A is a group of the Formula (A-9): 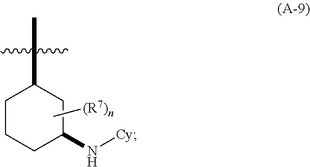
wherein [symbol] represents the point of attachment of A to the remainder of the molecule;
Cy is a ring selected from 5-10 membered heteroaryl optionally substituted with 1, 2, 3, 4, or 5 substituents independently selected from R^{Cy};
each R^{Cy} is independently selected from C₁₋₆ alkyl, C₃₋₁₀ cycloalkyl, 4-12 membered heterocycloalkyl, halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NOR^{a2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R⁷ is selected from D, C₁₋₆ alkyl, C₁₋₆ haloalkyl, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NOR^{a4})R^{b4}, C(═NR^{e4})NR^{c4}R^{d4}, NR^{c4}C(═NR^{e4})NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};
n is an integer from 0 to 8;
each R^{a1}, R^{c1}, R^{d1}, R^{a2}, R^{c2}, R^{d2}, R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
each R^{b1}, R^{b2}, and R^{b4} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
each R^{e2} and R^{e4} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{g1} is independently selected from C₁₋₆ alkyl, C₂₋₆ alkenyl, C₂₋₆ alkynyl, and R^{g2}, wherein the C₁₋₆ alkyl, C₂₋₆ alkenyl, and C₂₋₆ alkynyl are each optionally substituted with 1, 2, or 3 substituents independently selected from R^{g2};
each R^{g2} is independently selected from C₃₋₁₀ cycloalkyl, 4-10 membered heterocycloalkyl, C₆₋₁₀ aryl, 5-10 membered heteroaryl, halo, CN, OR^{a8}, SR^{a8}, C(O)R^{b8}, C(O)NR^{c8}R^{d8}, C(O)OR^{a8}, NR^{c8}R^{d8}, NR^{c8}C(O)R^{b8}, NR^{c8}C(O)OR^{a8}, NR^{c8}S(O)R^{b8}, NR^{c8}S(O)₂R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8};
each R^{a8}, R^{c8}, and R^{d8} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
each R^{b8} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl;
wherein at each occurrence, a heterocycloalkyl group has at least one ring-forming carbon atom and 1, 2, 3, or 4 ring-forming heteroatoms independently selected from N, O, and S; the N and S ring-forming heteroatoms of the heterocycloalkyl group are optionally oxidized; and a ring-forming carbon atom of the heterocycloalkyl group is optionally substituted by oxo to form a carbonyl group; and
at each occurrence, a heteroaryl group has at least one ring-forming carbon atom and 1, 2, 3, or 4 ring-forming heteroatoms independently selected from N, O, and S; the N and S ring-forming heteroatoms of the heteroaryl group are optionally oxidized; and a ring-forming carbon atom of the heteroaryl group is optionally substituted by oxo to form a carbonyl group;
with the proviso that the compound is not one of the following compounds:
N¹-[3-(2,6-Dichloro-4-methoxyphenyl)-2,5-dimethylpyrazolo[1,5-a]pyrimidin-7-yl]-N³-(tetrahydro-2H-pyran-4-yl)-1,3-cyclohexanediamine; and
N¹-[3-(2,6-Dichloro-4-methoxyphenyl)-2,5-dimethylpyrazolo[1,5-a]pyrimidin-7-yl]-N³-[1-(phenylmethyl)-4-piperidinyl]-1,3-cyclohexanediamine.

78. The compound or pharmaceutically acceptable salt thereof of claim 1, wherein the compound is selected from the following:
(1R,3S)—N¹-(3-methyl-3H-imidazo[4,5-c]pyridin-4-yl)-N³-(2-(trifluoromethyl)imidazo[1,2-a]pyridin-5-yl)cyclohexane-1,3-diamine;
(1s,4s)-N¹-(3-methyl-3H-imidazo[4,5-c]pyridin-4-yl)-N⁴-(2-(trifluoromethyl)imidazo[1,2-a]pyridin-5-yl)cyclohexane-1,4-diamine;
(1R,3S)—N¹-(1,6-naphthyridin-5-yl)-N³-(2-(trifluoromethyl)imidazo[1,2-a]pyridin-5-yl)cyclohexane-1,3-diamine;
(1R,3S)—N¹-(imidazo[1,5-a]pyrazin-8-yl)-N³-(2-(trifluoromethyl)imidazo[1,2-a]pyridin-5-yl)cyclohexane-1,3-diamine;
(1s,4s)-N¹-(imidazo[1,5-a]pyrazin-8-yl)-N⁴-(2-(trifluoromethyl)imidazo[1,2-a]pyridin-5-yl)cyclohexane-1,4-diamine;
(1R,3S)—N¹-(3-methyl-3H-imidazo[4,5-c]pyridin-4-yl)-N³-(2-methyl-5-(trifluoromethyl)pyrazolo[1,5-a]pyrimidin-7-yl)cyclohexane-1,3-diamine;
(1S,3R)—N¹-(2-methyl-5-(trifluoromethyl)pyrazolo[1,5-a]pyrimidin-7-yl)-N³-(1,6-naphthyridin-5-yl)cyclohexane-1,3-diamine;
(1S,3R)—N¹-(2-bromo-5-(trifluoromethyl)pyrazolo[1,5-a]pyrimidin-7-yl)-N³-(pyrazolo[1,5-a]pyrazin-4-yl)cyclohexane-1,3-diamine;
(1S,3R)—N¹-(2-bromo-5-(trifluoromethyl)pyrazolo[1,5-a]pyrimidin-7-yl)-N³-(1,6-naphthyridin-5-yl)cyclohexane-1,3-diamine;
(1R,3S)—N¹—([1,2,4]triazolo[4,3-a]pyridin-3-yl)-N³-(2-bromo-5-(trifluoromethyl)pyrazolo[1,5-a]pyrimidin-7-yl)cyclohexane-1,3-diamine;
(1S,3R)—N¹-(2,5-bis(trifluoromethyl)pyrazolo[1,5-a]pyrimidin-7-yl)-N³-(1,6-naphthyridin-5-yl)cyclohexane-1,3-diamine;
(1S,3R)—N¹-(2-bromo-5-(trifluoromethyl)pyrazolo[1,5-a]pyrimidin-7-yl)-N³-(imidazo[1,5-a]pyrazin-8-yl)cyclohexane-1,3-diamine;
(1S,3R)—N¹-(2-bromo-5-(trifluoromethyl)pyrazolo[1,5-a]pyrimidin-7-yl)-N³-(3-methyl-3H-imidazo[4,5-c]pyridin-4-yl)cyclohexane-1,3-diamine;
(1S,3R)—N¹-(2,5-bis(trifluoromethyl)pyrazolo[1,5-a]pyrimidin-7-yl)-N³-(imidazo[1,5-a]pyrazin-8-yl)cyclohexane-1,3-diamine;
(1S,3R)—N¹-(2,5-bis(trifluoromethyl)pyrazolo[1,5-a]pyrimidin-7-yl)-N³-(3-methyl-3H-imidazo[4,5-c]pyridin-4-yl)cyclohexane-1,3-diamine;
(1S,3R)—N¹-(2-bromo-5-(trifluoromethyl)pyrazolo[1,5-a]pyrimidin-7-yl)-N³-(imidazo[1,2-c]pyrimidin-5-yl)cyclohexane-1,3-diamine;
(1S,3R)—N¹-(2,5-bis(trifluoromethyl)pyrazolo[1,5-a]pyrimidin-7-yl)-N³-(imidazo[1,2-c]pyrimidin-5-yl)cyclohexane-1,3-diamine; and
(1R,3S)—N¹—([1,2,4]triazolo[4,3-a]pyridin-3-yl)-N³-(2,5-bis(trifluoromethyl)pyrazolo[1,5-a]pyrimidin-7-yl)cyclohexane-1,3-diamine.

79. A pharmaceutical composition comprising a compound or pharmaceutically acceptable salt thereof of claim 1 and a pharmaceutically acceptable carrier or excipient.

80. A method of treating an MRGPRX2 dependent condition in a patient comprising administering to the patient a therapeutically effective amount of a compound or pharmaceutically acceptable salt thereof of claim 1.

81. The method of claim 80, wherein the MRGPRX2 dependent condition is an itch associated condition, a pain associated condition, a pseudo-allergic reaction, an autoimmune or inflammatory disorder, or a cancer or tumor associated condition.

82. The method of claim 81, wherein the MRGPRX2 dependent condition is an itch associated condition.

83. The method of claim 82, wherein the MRGPRX2 dependent condition is an itch associated condition selected from the group consisting of chronic itch; senile itch; contact dermatitis; allergic blepharitis; anaphylaxis; anaphylactoid drug reactions; anaphylactic shock; anemia; atopic dermatitis; bullous pemphigoid; candidiasis; chicken pox; end-stage renal failure; hemodialysis; cholestatic pruritus; chronic spontaneous urticaria; chronic inducible urticaria; contact dermatitis, dermatitis herpetiformis; diabetes; drug allergy, dry skin; dyshidrotic dermatitis; ectopic eczema; eosinophilic fasciitis; epidermolysis bullosa; erythrasma; food allergy; folliculitis; fungal skin infection; hemorrhoids; herpes; HIV infection; hodgkin's disease; hyperthyroidism; iodinated contrast dye allergy; iron deficiency anemia; kidney disease; leukemia, porphyrias; lymphoma; mast cell activation syndrome, malignancy; mastocystosis; multiple myeloma; neurodermatitis; onchocerciasis; Paget's disease; pediculosis; polycythemia rubra vera; prurigo nodularis; lichen planus; lichen sclerosis; pruritus ani; pseudo-allergic reactions; pseudorabies; psoriasis; rectal prolapse; sarcoidosis granulomas; scabies; schistosomiasis; scleroderma, severe stress, stasia dermatitis; swimmer's itch; thyroid disease; tinea cruris; uremic pruritus; rosacea; cutaneous amyloidosis; scleroderma; acne; wound healing; burn healing; ocular itch; and urticaria.

84. The method of claim 81, wherein the MRGPRX2 dependent condition is a pain associated condition.

85. The method of claim 84, wherein the MRGPRX2 dependent condition is a pain associated condition selected from the group consisting of acute pain, advanced prostate cancer, AIDS-related pain, ankylosing spondylitis, arachnoiditis, arthritis, arthrofibrosis, ataxic cerebral palsy, autoimmune atrophic gastritis, avascular necrosis, back pain, Behcet's disease (syndrome), burning mouth syndrome, bursitis, cancer pain, carpal tunnel, cauda equina syndrome, central pain syndrome, cerebral palsy, cervical stenosis, Charcot-Marie-Tooth (CMT) disease, chronic fatigue syndrome (CFS), chronic functional abdominal pain (CFAP), chronic pain, chronic pancreatitis, chronic pelvic pain syndrome, collapsed lung (pneumothorax), complex regional pain syndrome (CRPS), reflex sympathetic dystrophy syndrome (RDS), corneal neuropathic pain, Crohn's disease, degenerative disc disease, dental pain, Dercum's disease, dermatomyositis, diabetic peripheral neuropathy (DPN), dystonia, Ehlers-Danlos syndrome (EDS), endometriosis, eosinophilia-myalgia syndrome (EMS), erythromelalgia, fibromyalgia, gout, headaches, herniated disc, hydrocephalus, intercostal neuralgia, interstitial cystitis, irritable bowel syndrome (IBS), juvenile dermatositis (dermatomyositis), knee injury, leg pain, loin pain-haematuria syndrome, lupus, Lyme disease, medullary sponge kidney (MSK), meralgia paresthetica, mesothelioma, migraine, musculoskeletal pain, myofascial pain, myositis, neck pain, neuropathic pain, occipital neuralgia, osteoarthritis, Paget's disease, pain crisis in sickle cell disease; Parsonage-Turner syndrome, pelvic pain, periodontitis pain, peripheral neuropathy, phantom limb pain, pinched nerve, polycystic kidney disease, polymyalgia rheumatica, polymyositis, porphyria, post herniorrhaphy pain syndrome, post mastectomy pain, postoperative pain, pain syndrome, post stroke pain, post thoracotomy pain syndrome, postherpetic neuralgia (shingles), post-polio syndrome, primary lateral sclerosis, psoriatic arthritis, pudendal neuralgia, radiculopathy, raynaud's disease, rheumatoid arthritis (RA), sacroiliac joint dysfunction, sarcoidosis, Scheuermann's kyphosis disease, sciatica, scoliosis, shingles (herpes zoster), Sjögren's syndrome, spasmodic torticollis, sphincter of oddi dysfunction, spinal cerebellum ataxia (SCA ataxia), spinal cord injury, spinal stenosis, syringomyelia, Tarlov cysts, transverse myelitis, trigeminal neuralgia, neuropathic pain, ulcerative colitis, vascular pain and vulvodynia.

86. The method of claim 81, wherein the MRGPRX2 dependent condition is a pseudo-allergic reaction.

87. The method of claim 86, wherein the MRGPRX2 dependent condition is a pseudo-allergic reaction caused by a secretagogue, cationic peptidergic drug, anionic peptidergic drug, neutral peptidergic drug, non-steroidal antiinflammatory drugs, neuropeptides, antimicrobial peptides, opioids, neuromuscular blocking agents, antidepressant agents, antipsychotic agents, antihistamine agents, antineoplastic agents, fluoroquinolone and non-fluoroquinolone antibiotics and tyrosine-kinase inhibitors.

88. The method of claim 86, wherein the MRGPRX2 dependent condition is a pseudo-allergic reaction caused by MCD peptide, substance P, VIP, PACAP, dynorphin, somatostatin, Compound 48/80, cortistatin-14, mastoparan, melittin, a cathelicidin peptide, ciprofloxacin, vancomycin, leuprolide, goserelin, histrelin, triptorelin, cetrorelix, ganirelix, degarelix, octreotide, lanreotide, pasireotide, sermorelin, tesamorelin, icatibant, glatiramer acetate, teriparatide, pramlintide, bleomycin, exenatide, glucagon, liraglutide, enfuvirtide, colistimethate, succinylcholine, tubocurarine, atracurium, mivacurium, or rocuronium.

89. The method of claim 81, wherein the MRGPRX2 dependent condition is an autoimmune or inflammatory disorder.

90. The method of claim 89, wherein the MRGPRX2 dependent condition is an autoimmune or inflammatory disorder selected from as chronic inflammation, mast cell activation syndrome, multiple sclerosis, Steven Johnson's syndrome, toxic epidermal necrolysis, appendicitis, bursitis, cutaneous lupus, colitis, cystitis, dermatitis, phlebitis, reflex sympathetic dystrophy/complex regional pain syndrome (RSD/CRPS), rhinitis, tendonitis, tonsillitis, acne vulgaris, sinusitis, rosacea, psoriasis, graft-versus-host disease, reactive airway disorder, asthma, airway infection, allergic rhinitis, autoinflammatory disease, celiac disease, chronic prostatitis, diverticulitis, glomerulonephritis, hidradenitis suppurativa, hypersensitivities, intestinal disorder, epithelial intestinal disorder, inflammatory bowel disease, irritable bowel syndrome, Crohn's disease, ulcerative colitis, lupus erythematous, interstitial cystitis, otitis, pelvic inflammatory disease, endometrial pain, reperfusion injury, rheumatic fever, rheumatoid arthritis, sarcoidosis, transplant rejection, psoriasis, lung inflammation, chronic obstructive pulmonary disease, permanent sputum eosinophilia, eosinophilic leukemia, eosinophilic esophagitis, eosinophilic gastritis, eosinophilic duodenitis, eosinophilic gastroenteritis, mast cell gastrointestinal disease, hypereosinophilic syndrome, aspirin-exacerbated respiratory disease, nasal polyposis, chronic rhinosinusitis, antibody-dependent cell-mediated cytotoxicity, neurofibromatosis, schwannomatosis, tubulointerstitial nephritis, glomerulonephritis, diabetic nephropathy, allograft rejection, amyloidosis, renovascular ischemia, reflux nephropathy, polycystic kidney disease, liver fibrosis/cirrhosis, autoimmune liver disease, biliary atresia, acute and chronic hepatitis B and C virus, liver tumors and cancers, alcoholic liver disease, polycystic liver disease, liver cholangiocarcinoma, primary sclerosing cholangitis, primary biliary cholangitis, neuromyelitis optica spectrum disorder, cardiovascular disease, inflammation induced by bacterial or viral infection, inflammation associated with SARS-COV-2 infection or its variants or coronavirus disease 2019 (COVID-19), acute respiratory distress syndrome, pneumonia, long/long-term/chronic COVID, postacute sequelae of COVID-19 (PASC), myalgic encephalomyelitis, chronic fatigue syndrome and vasculitis

91. The method of claim 81, wherein the MRGPRX2 dependent condition is a cancer or tumor associated condition.

92. The method of claim 91, wherein the MRGPRX2 dependent condition is a cancer or tumor associated condition selected from as adenoid cystic carcinoma, adrenal gland tumor, amyloidosis, anal cancer, appendix cancer, astrocytoma, ataxia-telangiectasia, Beckwith-Wiedemann syndrome, cholangiocarcinoma, Birt-Hogg-Dubé syndrome, bone cancer, brain stem glioma, brain tumor, breast cancer (inflammatory, metastatic, male), prostrate, basal cell, melanoma, colon, colorectal, bladder, kidney cancer, lacrimal gland cancer, laryngeal and hypopharyngeal cancer, lung cancer (non-small cell, small cell), leukemia (acute lymphoblastic, acute lymphocytic, acute myeloid, B cell prolymphocytic, chronic lymphocytic, chronic myeloid, chronic T cell lymphocytic, eosinophilic), liver cancer, Li-Fraumeni syndrome, lymphoma (Hodgkin and non-Hodgkin), lynch syndrome, mastocytosis, medulloblastoma, meningioma, mesothelioma, multiple endocrine neoplasia, multiple myeloma, MUTYH-associated polyposis, myelodysplastic syndrome, nasal cavity and paranasal sinus cancer, neuroblastoma, neuroendocrine tumors, neurofibromatosis, penile cancer, parathyroid cancer, ovarian fallopian tube and peritoneal cancer, osteosarcoma, pituitary gland tumor, pleuropulmonary blastoma, oral and oropharyngeal, thyroid, uterine, pancreatic, carney complex, brain and spinal cord cancer, cervical cancer, Cowden syndrome, craniopharyngioma, desmoid tumor, desmoplastic infantile ganglioglioma, ependymoma, esophageal cancer, Ewing sarcoma, eye cancer, eyelid cancer, familial adenomatous polyposis, familial GIST, familial malignant melanoma, familial pancreatic cancer, gallbladder cancer, gastrointestinal stromal tumor, germ cell tumor, gestational trophoblastic disease, head and neck cancer, hereditary breast and ovarian cancer, hereditary diffuse gastric cancer, hereditary, leiomyomatosis and renal cell cancer, hereditary pancreatitis, hereditary papillary renal carcinoma, hereditary mixed polyposis syndrome, HIV/AIDS related cancers, retinoblastoma, rhabdomyosarcoma, salivary gland cancer, Kaposi sarcoma, small bowel cancer, stomach cancer, testicular cancer, thymoma and thymic carcinoma, thyroid cancer, vaginal cancer, culver cancer, Wermer's syndroeroderma pigmentosum.

93. The method of claim 80, wherein the MRGPRX2 dependent condition is selected from abdominal aortic aneurysms, acute contact dermatitis, allergic rhinitis, amyotrophic lateral sclerosis, asthma, atopic dermatitis, autism, cancer, chronic inducible urticaria, chronic itch, chronic obstructive pulmonary disease, chronic spontaneous urticaria, cold urticaria, contact urticaria, coronary artery disease, cough, Crohn's disease, deep vein thrombosis, drug-induced anaphylactic reactions, endometriosis, fibromyalgia, geographic atrophy, idiopathic chronic cough, idiopathic pulmonary fibrosis, inflammatory pain, interstitial cystitis, irritable bowel syndrome, mast cell activation syndrome, mastocytosis, metabolic syndrome, migraine, multiple sclerosis, nasal polyps, neurodermatitis, neuropathic itch, neuropathic pain, obesity, oesophageal reflux, osteoarthritis, periodontitis, prurigo nodularis, pruritus, pseudo-anaphylaxis, psoriasis, rheumatoid arthritis, rosacea, seborrheic dermatitis, sickle cell disease, ulcerative colitis, and ulcers.

94. The method of claim 80, wherein the MRGPRX2 dependent condition is selected from autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, graft-versus-host disease (GvHD), Guillain-Barre syndrome, Hashimoto's encephalitis, Hashimoto's thyroiditis, ankylosing spondylitis, psoriasis, Sjögren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

95. The method of claim 80, wherein the MRGPRX2 dependent condition is selected from acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/Anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticaria, axonal or neuronal neuropathies, Balo disease, Behcet's disease, bullous pemphigoid, cardiomyopathy, Castleman disease, celiac disease, Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibromyalgia, fibrosing alveolitis, giant cell arteritis (temporal arteritis), giant cell myocarditis, glomerulonephritis, Goodpasture's syndrome, granulomatosis with polyangiitis (GPA) (formerly called Wegener's granulomatosis), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, Immunoregulatory lipoproteins, inclusion body myositis, interstitial cystitis, juvenile arthritis, juvenile diabetes (type 1 diabetes), juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (SLE), Lyme disease, chronic, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (pediatric autoimmune neuropsychiatric disorders associated with streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatica, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm & testicular autoimmunity, stiff person syndrome, subacute bacterial endocarditis (SBE), Susac's syndrome, sympathetic ophthalmia, takayasu's arteritis, temporal arteritis/giant cell arteritis, thrombocytopenic purpura (TTP), tolosa-hunt syndrome, transverse myelitis, type 1 diabetes, ulcerative colitis, undifferentiated connective tissue disease (UCTD), uveitis, vasculitis, vesiculobullous dermatosis, vitiligo, and Wegener's granulomatosis (i.e., granulomatosis with polyangiitis (GPA)).
